(19) United States
(12) Patent Application Publication
(10) Pub. No.: US 2025/0302929 A1
(43) Pub. Date: Oct. 2, 2025

(54) TUMOR NEOANTIGENIC PEPTIDES

(71) Applicants: INSTITUT CURIE, Paris (FR); MNEMO THERAPEUTICS, Paris (FR); INSERM (INSTITUT NATIONAL DE LA SANTE ET DE LA RECHERCHE MEDICALE), Paris (FR)

(72) Inventors: Sebastian AMIGORENA, Paris (FR); Marianne BURBAGE, Paris (FR); Alexandre HOUY, Vitry sur Seine (FR); Joshua WATERFALL, Paris (FR); Marc-Henri STERN, Paris (FR); Benjamin SADACCA, Paris (FR); Antonela MERLOTTI IPPOLITO, Paris (FR); Yago ARRIBAS DE SANDOVAL, Paris (FR); Christel GOUDOT, Le Blanc Mesnil (FR)

(21) Appl. No.: 18/549,993

(22) PCT Filed: Mar. 11, 2022
(86) PCT No.: PCT/EP2022/056332

§ 371 Date: Sep. 11, 2023

(30) Foreign Application Priority Data
Mar. 11, 2021 (EP) 21305299.6
Mar. 2, 2022 (EP) 22305237.4

(51) Int. Cl. A61K 39/00; A61P 35/00; C07K 14/82

(52) U.S. Cl. CPC A61K 39/0011; A61P 35/00; C07K 14/82

(57) ABSTRACT

The present disclosure provides tumor neoantigenic peptide sequences and nucleotide sequences encoding such peptide sequences; a vaccine or immunogenic composition capable of raising a specific T-cell response comprising one or more of the neoantigenic peptides, or comprising nucleic acid encoding one or more of the neoantigenic peptides; an antibody, or an antigen-binding fragment thereof, a T cell receptor (TCR), or a chimeric antigen receptor (CAR) that specifically binds such neoantigenic peptides; methods of producing such antibodies, TCRs or CARs; polynucleotides encoding such neoantigenic peptides, antibodies, CARs or TCRs, optionally linked to a heterologous regulatory control sequence; immune cells that specifically bind to such neoantigenic peptides; and dendritic cells or antigen presenting cells that have been pulsed with one or more of the neoantigenic peptides; and methods of using such products in particular therapeutic uses of these products.

### RELATED APPLICATIONS

This application is the U.S. national phase of International Patent Application No. PCT/EP2022/056332, filed Sep. 11, 2022, which claims priority to European Patent Application Nos. 21305299.6, filed Mar. 11, 2021, and 22305237.4, filed Mar. 2, 2022, both of which are hereby incorporated by reference in their entirety.

### INCORPORATION BY REFERENCE OF SEQUENCE LISTING

This application includes, as a separate part of disclosure, a Sequence Listing in computer-readable form (Filename: SequenceListing.txt, Size: 91,513,022 bytes; Created: Mar. 27, 2024). The contents of the Sequence Listing text file are incorporated herein by reference in its entirety.

### FIELD OF THE DISCLOSURE

The present disclosure provides neoantigenic peptides encoded by transposable element (TE)-exon fusion transcripts, nucleic acids, vaccines, antibodies and immune cells that can be used in cancer therapy.

### BACKGROUND

Harnessing the immune system to generate effective responses against tumors is a central goal of cancer immunotherapy. Part of the effective immune response involves T lymphocytes specific for tumor antigens. T cell activation requires their interaction with antigen-presenting cells (APCs), commonly dendritic cells (DCs), expressing TCR-cognate peptides presented in the context of a major histocompatibility molecule (MHC) and co-stimulation signals. Subsequently, activated T cells can recognize peptide-MHC complexes presented by all cell types, even malignant cells. Neoplasms often contain infiltrating T lymphocytes reactive with tumor cells.

However, the efficiency of immune responses against tumors is severely dampened by various immunosuppressive strategies developed by tumors; e.g, tumor cells express receptors that provide inhibitory signals to infiltrating T cells, or they secrete inhibitory cytokines. The development of checkpoint blockade therapy has provided means to bypass some of these mechanisms, leading to more efficient killing of cancer cells. The promising results yielded by this approach have opened up new avenues for the development of T cell-based immunotherapy. Checkpoint inhibitors are, however, effective in a minority of patients and only in limited types of cancer.

A major goal in immunotherapy is to increase the proportion of responding patients and extend the cancer indications. Vaccination, administration of anti-tumor antibodies, or administration of immune cells specific for tumor antigens have all been proposed to increase the anti-tumor immune response, and can be administered alone, with other therapies such as chemotherapy or radiation, or as a combination therapy with checkpoint blockers. The selection of antigens able to trigger anti-tumor immunity without targeting healthy tissues has been a long-standing challenge.

The search for tumor neoantigens has mostly been focused on mutated sequences appearing as in cancer cells. These antigens are unique to each patient. Tumor antigens (the ones preferentially expressed in tumor cells) are, however, self-antigens that represent poor targets for vaccination (probably due to central tolerance). Identifying shared true neoantigens (absent from tissues) is a major challenge for the field.

A few prior reports regarding transposable elements (TE) in tumors include (Helman, E. et al. (2014). Genome Res.) (Schiavetti, F. et al. (2002). Cancer Res., Takahashi, Y. et al. (2008). J. Clin. Invest.). (Chiappinelli, K. B. et al. (2015). Cell, Roulois, D. et al. (2015). Cell). However, the relationship of TE to the antigenic landscape presented by tumor cells has not been investigated in depth.

New tumor neoantigens would be of interest and might improve or reduce the cost of cancer therapy in particular in the case of vaccination and adoptive cell therapy.

### SUMMARY

The inventors have now discovered that non-canonical alternative splicing events between exons and TEs, also named herein JETs (junctions between TEs and exons) can be a source of tumor antigens and more particularly a source of tumor-specific antigens. The present disclosure therefore provides tumor neoantigenic peptide sequences and nucleotide sequences encoding such peptide sequences; a vaccine or immunogenic composition capable of raising a specific T-cell response comprising one or more of the neoantigenic peptides, or comprising nucleic acid encoding one or more of the neoantigenic peptides; an antibody, or an antigen-binding fragment thereof, a T cell receptor (TCR), or a chimeric antigen receptor (CAR) that specifically binds such neoantigenic peptides; methods of producing such antibodies, TCRs or CARs; polynucleotides encoding such neoantigenic peptides, antibodies, CARs or TCRs, optionally linked to a heterologous regulatory control sequence; immune cells that specifically bind to such neoantigenic peptides; and dendritic cells or antigen presenting cells that have been pulsed with one or more of the neoantigenic peptides; and methods of using such products. The present disclosure provides tumor neoantigenic peptides comprising at least 8 amino acids, optionally wherein said neoantigenic peptides are encoded by a part of an open reading frame (ORF) from a fusion transcript sequence comprising a transposable element (TE) sequence and an exonic sequence. While in some embodiments, the tumor neoantigenic peptide is at least 8 amino acids in length, and/or up to about 25 amino acids in length, antibodies, TCRs or CARs that specifically bind the neoantigenic peptide may bind a peptide sequence of at least 4, at least 5, at least 6, or at least 7 amino acids.

According to the present disclosure, “neoantigen peptide characteristics” include neoantigenic peptide derived from fusion transcripts (splicing variants) wherein:
the TE sequence can be located in 5′ end of the fusion transcript sequence and the exonic sequence can be located in 3′ end of the fusion transcript sequence, and the part of the ORF of said fusion transcript sequence, which encodes the neoantigenic peptide, can overlap the junction;
the TE sequence can be located in 5′ end of the fusion transcript sequence and the exonic sequence can be located in 3′ end of the fusion transcript sequence, and the part of ORF which encodes said tumor neoantigenic peptide, can be downstream of the junction such that the open reading frame is non-canonical;
the TE sequence can be located in 3′ end of the fusion transcript sequence and the exonic sequence can be located in 5′ end of the fusion transcript sequence and the part of the ORF of said fusion transcript sequence, which encodes the tumor neoantigenic peptide, can overlap the junction; or
the TE sequence is located in 3′ end of the fusion transcript sequence and the exonic sequence is located in 5′ end of the chimeric transcript sequence, the part of the ORF which encodes a tumor neoantigenic peptide, is downstream of the junction between the exonic sequence and the TE sequence, optionally wherein the peptide sequence which is thus encoded by the pure TE sequence is non-canonical.

The TE sequences can be selected from the TE class I: Endogenous Retro Virus (ERVs), Long interspersed nuclear elements (LINEs) and short interspersed nuclear element (SINEs) and MaLR sequences or the DNA transposons of class II.

The present disclosure notably provides an isolated tumor neoantigenic peptide comprising at least 8 amino acids of SEQ ID NO:1-38907 and 38916-41099 and optionally comprising a transposable element (TE) sequence and an exonic sequence, wherein said ORF overlaps the junction between the TE and the exonic sequence, is pure TE and/or is non-canonical.

Most particularly, the present disclosure provides an isolated tumor neoantigenic peptide, according to claim 1 wherein the peptide is from any one of SEQ ID NO: 1-38907 and 38916-41099, including a fragment thereof, and comprises at least a portion of a TE-derived amino acid sequence or is from any one of SEQ ID NO:1-38907 and 38916-41099. In some embodiments, the neoantigenic peptide overlaps the breakpoint between, the TE-derived amino acid sequence and the exon-derived amino acid sequence. In other embodiments, the neoantigenic peptide is derived from a pure TE sequence. In yet other embodiments, the neoantigenic peptide is encoded by a non-canonical ORF downstream of the junction between the TE-derived amino acid sequence and the exon-derived amino acid sequence.

In one embodiment, the tumor neoantigenic peptide is 8 or 9 amino acids long, notably 8 to 11, and binds to at least one MHC class I molecule.

In another embodiment, the tumor neoantigenic peptide is from 13 to 25 amino acids long, and binds to at least one MHC class II molecule of said subject.

Said neoantigenic peptides are typically expressed at higher levels, or higher frequency, in tumor samples compared to normal, optionally said neoantigenic peptides are not expressed in normal tissue samples (i.e. normal healthy cells), or not detectably expressed in normal healthy samples.

In some embodiments said neoantigenic peptides are expressed in at least 1%, 5%, 10%, 15%, 20% 25% or even at least 30% of subjects from a population of subjects suffering from cancer and notably from a population of subjects suffering from cancer, notably from lung cancer, more particularly Non-small cell lung cancer (NSCLC), even more particularly from lung adenocarcinoma (LUAD).

Typically, the neoantigenic peptides bind MHC class I or class II with a binding affinity Kd of less than about 10⁻⁴, 10⁻⁵, 10⁻⁶, 10⁻⁷, 10⁻⁸ or 10⁻⁹ M (lower numbers indicating higher binding affinity).

Typically, the neoantigenic peptides bind MHC class I with a binding affinity of less than 2% percentile rank score predicted by NetMHCpan 4.0

Typically, the neoantigenic peptides bind MHC class II with a binding affinity of less than 10% percentile rank score predicted by NetMHCpanII 3.2.

The present disclosure also encompasses:
a population of autologous dendritic cells or antigen presenting cells that have been pulsed with one or more of the peptides as herein defined, or transfected with a polynucleotide encoding one or more of the peptides as herein described;
a vaccine or immunogenic composition, notably a sterile vaccine or immunogenic composition, capable of raising a specific T-cell response comprising
   a. one or more neoantigenic peptides as herein defined,
   b. one or more polynucleotides encoding a neoantigenic peptide as herein defined, optionally wherein the one or more polynucleotides are linked to a heterologous regulatory control nucleotide sequence; or
   c. a population of autologous dendritic cells or antigen presenting cells (notably artificial APC) that have been pulsed or loaded with one or more of the peptides as herein defined,
optionally in combination with a physiologically or pharmacologically acceptable buffer, carrier, excipient, immunostimulant and/or adjuvant.
an antibody, or an antigen-binding fragment thereof, a T cell receptor (TCR), or a chimeric antigen receptor (CAR) that has been selected for its binding affinity to a neoantigenic peptide from any one of SEQ ID NO: 1-38907 and 38916-41099, including a portion thereof, e.g. of a length at least 4, 5, 6 7, or 8 amino acids, or a composition comprising such antibody, antigen-binding fragment thereof, TCR or CAR.
a polynucleotide encoding a neoantigenic peptide, an antibody, a CAR or a TCR as herein defined, typically operatively linked to a heterologous regulatory control nucleotide sequence, and a vector encoding such polynucleotide, or a vaccine or immunogenic composition comprising such polynucleotide or vector;
an immune cell, or a population or immune cells that targets one or more neoantigenic peptides from any one of SEQ ID NO:1-38907 and 38916-41099, including a portion thereof, e.g. of a length at least 4, 5, 6 7, or 8 amino acids, wherein the population of immune cells preferably targets a plurality of different tumor neoantigenic peptides as herein disclosed, or a composition comprising such immune cells or population of immune cells optionally in combination with a physiologically or pharmacologically acceptable buffer, carrier, excipient, immunostimulant and/or adjuvant.

Typically, the antibody or antigen-binding fragment thereof, TCR or CAR binds a neoantigenic peptide, optionally in association with an MHC molecule, or optionally expressed on the surface of a cell, with a Kd affinity of about 10⁻⁶ M or less.

In some embodiments, the T cell receptor can be made soluble and fused to an antibody fragment directed to a T cell antigen, optionally wherein the targeted antigen is CD3 or CD16.

In some embodiments, the antibody can be a multispecific antibody that further targets at least an immune cell antigen, optionally wherein the immune cell is a T cell, a NK cell or a dendritic cell, optionally wherein the targeted antigen is CD3, CD16, CD30 or a TCR. In any of the embodiments relating to an antibody, the antibody can be chimeric, humanized, or human, and may be IgG, e.g. IgG1, IgG2, IgG3, IgG4.

The immune cell can be typically a T cell or a NK cell, a CD4+ and/or CD8+ cell, a TILs/tumor derived CD8 T cells, a central memory CD8+ T cells, a Treg, a MAIT, or a Yδ T cell. The cell can also be autologous or allogenic. Methods of preparing such immune cells are also contemplated, for example, by delivering a nucleic acid or vector encoding any of the antibody, TCR, or CAR described herein to the cell, in vivo or ex vivo.

The immune cell, e.g. T cell, can comprises comprise a recombinant antigen receptor selected from T cell receptor and chimeric antigen receptor as herein described, wherein the antigen is a tumor neoantigenic receptor as herein disclosed.

The present disclosure also encompasses a method of producing an antibody, TCR or CAR that specifically binds a neoantigenic peptide as herein described and comprising the step of selecting an antibody, TCR or CAR that binds to a tumor neoantigen peptide of the present disclosure, optionally in association with an MHC or HLA molecule, or optionally expressed on the surface of a cell, optionally with a Kd binding affinity of about 10⁻⁶ M or less. Antibodies, TCRs and CARs selected by said method are also part of the present application, and thus any references to antibodies, TCRs or CARs herein also means an antibody, TCR or CAR that has been selected by said method.

A polynucleotide encoding a neoantigenic peptide as herein defined, or encoding an antibody, a CAR or a TCR as herein defined, optionally linked to a heterologous regulatory control sequence are also part of the present application.

As per the present disclosure, the neoantigenic peptide, the population of dendritic cells, the vaccine or immunogenic composition, the polynucleotide or the vector encoding the peptide can be used in cancer vaccination therapy of a subject; or for treating cancer in a subject suffering from cancer or at risk of cancer; or can be used for inhibiting proliferation of cancer cells. Typically, the peptide(s) bind at least one MHC molecule of said subject. Treatment as used herein includes both prophylactic and therapeutic treatment.

As per the present disclosure, the antibody or the antigen-binding fragment thereof, the multispecific antibody, the TCR, the CAR, the polynucleotide, or the vector encoding such antibody, TCR or CAR, or the immune cells, as herein defined can be used in the treatment of cancer in a subject in need thereof, the subject suffering from cancer or at risk of cancer, or can be used for inhibiting proliferation of cancer cells. Still as per the present disclosure, the population of immune cells as herein defined can be used in cell therapy of a subject suffering from cancer or at risk of cancer, or can be used for inhibiting proliferation of cancer cells.

Pharmaceutical compositions comprising any of the foregoing, optionally with a sterile pharmaceutically acceptable excipient(s), carrier, and/or buffer are also contemplated as well as methods of using them.

In any of the embodiments described herein, the Cancer Therapeutic Products as above defined can be administered in combination with at least one further therapeutic agent. Such further therapeutic agent can typically be a chemotherapeutic agent, or an immunotherapeutic agent.

For example, according to the present disclosure, any of the Cancer Therapeutic Products can be administered in combination with an anti-immunosuppressive/immunostimulatory agent. For example, the subject is further administered with one or more checkpoint inhibitors typically selected from PD-1 inhibitors, PD-L1 inhibitors, Lag-3 inhibitors, Tim-3 inhibitors, TIGIT inhibitors, BTLA inhibitors, V-domain Ig suppressor of T-cell activation (VISTA) inhibitors and CTLA-4 inhibitors, or IDO inhibitors.

Various embodiments of the methods, neoantigenic peptides and Cancer Therapeutic Products are described in detailed below. Except for alternatives clearly mentioned, combinations of such embodiments are encompassed by the present application.

### DETAILED DISCLOSURE

Transposable elements (TEs) expression in normal tissues is silenced by DNA methylation established early during embryonic development. An additional layer of inhibition is provided by histone modifications. TEs can be re-activated in tumor cells. The inventors have discovered and provided clear evidence that non-canonical alternative splicing events between exons and TEs can be a source of tumor antigens, in particular of tumor-specific antigens.

The Inventors have developed a method for identifying a tumor antigen, and notably a tumor specific antigen. In particular, the inventors have identified a method for identifying tumor antigens derived from junctions between TEs and exons (JETs). In some embodiments, the present invention therefore relates to a method for identifying, or selecting, a tumor neoantigenic peptide encoded by a fusion transcript (i.e.: JET) sequence comprising a part of a TE sequence and a part of an exonic sequence.

The neoantigenic tumor specific peptides, in particular neoantigenic peptides from any one of SEQ ID NO:1-38759, identified by the method according to the present disclosure are highly immunogenic. Indeed, because they are derived from fusion transcripts (also named herein JETs), composed of a transposable element, TE, and an exonic sequence, which are absent from normal cells, the peptides of the present disclosure are expected to exhibit very low immunological tolerance.

The present disclosure also allows selecting peptides having shared tumor neoepitopes among a population of patients. Such shared tumor peptides are of high therapeutic interest since they may be used in immunotherapy for a large population of patients.

### Definitions

According to the present disclosure, the term “disease” refers to any pathological state, including cancer diseases, in particular those forms of cancer diseases described herein.

The term “normal” refers to the healthy state or the conditions in a healthy subject or tissue, i.e., non-pathological conditions, wherein “healthy” preferably means non-cancerous.

Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor but some, like leukemia, do not.

Malignant tumor is essentially synonymous with cancer. Malignancy, malignant neoplasm, and malignant tumor are essentially synonymous with cancer.

As used herein, the term “tumor” or “tumor disease” refers to an abnormal growth of cells (called neoplastic cells, tumorigenous cells or tumor cells) preferably forming a swelling or lesion. By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

A benign tumor is a tumor that lacks all three of the malignant properties of a cancer. Thus, by definition, a benign tumor does not grow in an unlimited, aggressive manner, does not invade surrounding tissues, and does not spread to non-adjacent tissues (metastasize).

Neoplasm is an abnormal mass of tissue as a result of neoplasia. Neoplasia (new growth in Greek) is the abnormal proliferation of cells. The growth of the cells exceeds, and is uncoordinated with that of the normal tissues around it. The growth persists in the same excessive manner even after cessation of the stimuli. It usually causes a lump or tumor. Neoplasms may be benign, pre-malignant or malignant.

“Growth of a tumor” or “tumor growth” according to the present disclosure relates to the tendency of a tumor to increase its size and/or to the tendency of tumor cells to proliferate.

For purposes of the present disclosure, the terms “cancer” and “cancer disease” are used interchangeably with the terms “tumor” and “tumor disease”.

Cancers are classified by the type of cell that resembles the tumor and, therefore, the tissue presumed to be the origin of the tumor. These are the histology and the location, respectively.

According to the present application, cancer may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the disclosure therefore comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the present disclosure also comprises cancer metastases and relapse of cancer.

The main types of lung cancer are small cell lung carcinoma (SCLC) and non-small cell lung carcinoma (NSCLC). There are three main sub-types of the non-small cell lung carcinomas: squamous cell lung carcinoma, lung adenocarcinoma (LUAD), and large cell lung carcinoma. Adenocarcinomas account for approximately 10% of lung cancers. This cancer usually is seen peripherally in the lungs, as opposed to small cell lung cancer and squamous cell lung cancer, which both tend to be more centrally located.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

The cells of a secondary or metastatic tumor are like those in the original tumor. This means, for example, that, if ovarian cancer metastasizes to the liver, the secondary tumor is made up of abnormal ovarian cells, not of abnormal liver cells. The tumor in the liver is then called metastatic ovarian cancer, not liver cancer.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, according to the present disclosure, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

By “treat” is meant to administer a compound or composition as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject. In particular, the term “treatment of a disease” includes curing, shortening the duration, ameliorating, preventing, slowing down or inhibiting progression or worsening, or preventing or delaying the onset of a disease or the symptoms thereof.

By “being at risk” is meant a subject, i.e. a patient, that is identified as having a higher than normal chance of developing a disease, in particular cancer, compared to the general population. In addition, a subject who has had, or who currently has, a disease, in particular cancer, is a subject who has an increased risk for developing a disease, as such a subject may continue to develop a disease. Subjects who currently have, or who have had, a cancer also have an increased risk for cancer metastases.

The therapeutically active agents, vaccines and compositions described herein may be administered via any conventional route, including by injection or infusion.

The agents described herein are administered in effective amounts. An “effective amount” refers to the amount which achieves a desired reaction or a desired effect alone or together with further doses or together with further therapeutic agents. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition.

An effective amount of an agent described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on various of such parameters. In the case that a reaction in a patient is insufficient with an initial dose, higher doses (or effectively higher doses achieved by a different, more localized route of administration) may be used.

The pharmaceutical compositions as herein described are preferably sterile and contain an effective amount of the therapeutically active substance to generate the desired reaction or the desired effect.

The pharmaceutical compositions as herein described are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g. CpG oligonucleotides, cytokines, chemokines, saponin, GM-CSF and/or RNA and, where appropriate, other therapeutically active compounds. When used in medicine, the salts should be pharmaceutically compatible.

As used herein, the term “nucleic acid molecules” include any nucleic acid molecule that encodes a polypeptide of interest or a fragment thereof. Such nucleic acid molecules need not be 100% homologous or identical with an endogenous nucleic acid sequence but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, e.g., at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. ETseful variations on these conditions will be readily apparent to those skilled in the art. For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., of at least about 42° C., or of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

Unless specifically stated or obvious from context, as used herein, the term “about” is to be understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

A “transposable element” as used herein is a repeated DNA sequence DNA sequences that is able to move from one location to another in the genome either through an RNA copy generated by a reverse transcriptase (Class I TEs, retrotransposons), or by excising themselves from their original location (Class II TEs, or DNA transposons). It thus includes both class I (retrotransposons, including those containing LTRs, LINEs and SINEs) and class II (DNA transposons) endogenously part of the genome (i.e.: not from infection). This includes both autonomous and non-autonomous elements from both classes. According to the present disclosure the TE sequences can be for example selected from TE of class I, such as retrotransposons including Endogenous RetroVirus (ERVs), Long interspersed nuclear elements (LINEs) and short interspersed nuclear element (SINEs) and mammalian long terminal repeat transposon (MaLR), and TE of class II, such as DNA transposons endogenously part of the genome.

Retrotransposons are by far more abundant and their characteristics are similar to retroviruses, such as HIV. Retrotransposons function via reverse transcription of an RNA intermediate replicative mechanism. They are commonly grouped into three main orders: retrotransposons with long terminal repeats (LTRs) flanking the retroelement main body, which encode reverse transcriptase, similar to retroviruses; retroposons with long interspersed nuclear elements (LINEs, LINE-1s, or L1s), which encode reverse transcriptase but lack LTRs, and are transcribed by RNA polymerase II; and retrotransposons with short interspersed nuclear elements (SINEs) that do not encode reverse transcriptase and are transcribed by RNA polymerase III. DNA transposons have a transposition mechanism that do not involve an RNA intermediate. The transpositions are catalyzed by several transposase enzymes. LTRs include endogenous retroviruses (ERVs), while non-LTR TEs subdivide into long-interspersed (LINEs) and short interspersed elements (SINEs), nonautonomous transposons mobilized by the LINE integration machinery. These lineages are composed of phylogenetically related families, further branching out into multiple subfamilies, each originating from one precursor copy. With time, the accumulation of mutations introduced divergence in the consensus sequence within members of each subfamily. For review on TE retrotransposon, see Richardson, Sandra R et al. “The Influence of LINE-1 and SINE Retrotransposons on Mammalian Genomes.” Microbiology spectrum vol. 3,2 (2015): MDNA3-0061-2014.

A typical L1 element is approximately 6,000 base pairs (bp) long and consists of two non-overlapping open reading frames (ORF) which are flanked by untranslated regions (UTR) and target site duplications. LINE-1 retrotransposons have been amplifying in mammalian genomes for greater than 160 million years. In humans, the vast majority of LINE-1 sequences have amplified since the divergence of the ancestral mouse and human lineages approximately 65-75 million years ago. Sequence comparisons between individual genomic LINE-1 sequences and a consensus sequence derived from modern, active LINE-1s can be used to estimate the age of genomic LINE-1s (Khan H, Smit A, Boissinot S; Genome Res. 2006 January; 16 (1): 78-87). L1 subfamilies typically categorize into old (L1M, AluJ), intermediate (L1P, L1PB, AluS), young (L1HS, L1PA, AluY) and related (HAL, FAM) subfamilies. In humans, the only autonomously active family is the long-interspersed element-1 (LINE-1 or L1), however a few L1 copies are still retrotransposition competent, all of them belonging to the youngest human-specific L1HS subfamily.

SVA elements comprise an evolutionarily young, non-autonomous retrotransposon family that arose in primate lineages approximately 25 million years ago (Hancks D C, Kazazian H H Jr, Semin Cancer Biol. 2010 August; 20 (4): 234-45). A typical SVA element is approximately 2,000 bp and has a composite structure that consists of: 1) a hexameric CCCTCT repeat; 2) an inverted Alu-like element repeat; 3) a set of GC-rich variable nucleotide tandem repeats (VNTRs); 4) a SINE-R sequence that shares homology with HERVK-10, an inactive LTR retrotransposon; and 5) a canonical cleavage polyadenylation specificity factor (CPSF) binding site that is followed by a poly (A) tract. The youngest SVA subfamilies include SVA-D, SVA-E, SVA-F, and SVA-F1 subfamilies.

A “messenger RNA (mRNA)” is a single-stranded RNA molecule that corresponds to the genetic sequence of a gene and is read by the ribosome in the process of producing a protein. mRNA is created during the process of transcription, where the enzyme RNA polymerase converts genes into primary transcript mRNA (also known as pre-mRNA). This pre-mRNA usually still contains introns, regions that will not go on to code for the final amino acid sequence. These are removed in the process of RNA splicing, leaving only exons, regions that will encode the protein. This exon sequence constitutes mature mRNA. Mature mRNA is then read by the ribosome, and, utilizing amino acids carried by transfer RNA (tRNA), the ribosome creates the peptide sequence a process called translation.

A “transcript” as herein intended is a messenger RNA (or mRNA) or a part of a mRNA which is expressed by an organism, notably in a particular tissue or even in a particular tissue. Expression of a transcript varies depending on many factors. Expression of a transcript may be modified in a cancer cell as compared to a normal healthy cell.

A “transcriptome” as herein intended is the full set of messenger RNA, or mRNA, molecules expressed or transcribed by the gene of a cell. In some embodiments, the term “transcriptome” can also be used to describe the array of mRNA transcripts produced in a particular cell (or tissue type). In contrast with the genome, which is characterized by its stability, the transcriptome actively changes. In fact, an organism's transcriptome varies depending on many factors, including stage of development, environmental and physiological conditions. Typically, also, the transcriptome is modified in a cancer cell as compared to a corresponding normal healthy cell. Typically, the transcriptome as herein intended is the human transcriptome. The terms “transcriptomic pattern” and “transcriptome” are used herein as synonyms.

A reading frame is a way of dividing the sequence of nucleotides in a nucleic acid (DNA or RNA) molecule into a set of consecutive, non-overlapping triplets.

An open reading frame (ORF) is the part of a reading frame that has the ability to be translated into a peptide. An ORF is a continuous stretch of codons that contain a start codon (for example AUG) at a transcription starting site (TSS) and a stop codon (for example UAA, UAG or UGA). An ATG codon within the ORF (not necessarily the first) may indicate where translation starts. The transcription termination site is located after the ORF, beyond the translation stop codon. In eukaryotic genes with multiple exons, ORFs span intron/exon regions, which may be spliced together after transcription of the ORF to yield the final mRNA for protein translation.

A “canonical ORF” as herein intended is a protein coding sequence with specified reading frame within a mRNA sequence which is described or annotated in databases such as for example Ensembl genome/transcriptome/proteome database collection (typically HG19). Typically, a canonical ORF is the same as one of the exons in normal healthy cells.

A “non-canonical ORF” as herein intended is a protein coding sequence with specified reading frame within a mRNA sequence which is not described (i.e. unannotated) in genome databases such as for example in Ensembl genome/transcriptome/proteome database. Typically, a non-canonical ORF means thus that the reading frame is shifted compared to the usual reading frame of exons in normal healthy cells. In some embodiments however, a non-canonical can be described in genome databases (such as Ensembl database), but the mRNA sequence represents minor species in normal cells. By minor species it is typically intended less that 5%, notably less than 2%, or preferentially less than 1% species in normal cells.

An exon is any part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed, and exons are covalently joined to one another as part of generating the mature messenger RNA. An exonic sequence as per the present applicant comprises at least a portion of one or more exons. Typically, the exonic sequence comprises at least a portion of one or 2 exons.

The untranslated sequences in 3′end and in 5′ end (3′UTR and 5′UTR) present in mature RNA after splicing are exonic sequences, but are non-coding sequences because these sequences are located upstream of the start codon for the translation (5′UTR) or downstream of the stop codon ending the translation (3′UTR).

In the present application, the terms “fusion transcript”, “chimeric transcripts” “TE-exon transcript”, or “Junction Exon-TE” (JET) are used indifferently as synonyms. A “fusion or a chimeric” “transcript or sequence”, as per the present disclosure is defined as a transcript that aligns in part with an exon sequence and in part with a transposable element (TE) sequence. A fusion, or chimeric, transcript is also shortly named herein JET (junction between exon and TE). Typically, a fusion transcript according to the present description has a normalized number of read greater than 2.10⁻⁶. The normalized number of reads is defined as the number of reads that cover the fusion divided by the library size of the sample.

The term “peptide or polypeptide,” is used interchangeably with “neoantigenic peptide or polypeptide” in the present specification to designate a series of residues, typically L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification does not destroy the biological activity of the polypeptides as herein described.

As used herein pJETs are peptides or polypeptides derived from (i.e. encoded by) chimeric/fusion transcripts or JETs. pJETs are also named herein translated JETs.

A “reference genome, or “representative genome” is a digital nucleic acid sequence data base, assembled by scientists as a representative example of species set of genes. As they are often assembled from the sequencing of DNA from a number of donors, reference genomes do not accurately represent the set of genes of any single individual (animal or person). Instead a reference provides a haploid mosaic of different DNA sequences from each donor.

RNA-Seq (named as an abbreviation of RNA sequencing) is a sequencing technique which uses next-generation sequencing (NGS) to reveal the presence and quantity of RNA (typically messenger RNA, mRNA) in a biological sample and generates an enormous numbers of raw sequencing reads (typically at least in the tens of millions). Single-cell RNA sequencing (scRNA-Seq) provides the expression profiles of an individual cell. A read refers to an RNA sequence from one RNA fragment from a biological sample or a single cell. The RNA sample that was sequenced is called the RNA library. RNA sequencing data are thus typically called RNA reads.

In the present application, “MHC molecule” or “HLA molecule” refers to at least one MHC/HLA class I molecule or at least one MHC/HLA Class II molecule. MHC class I proteins form a functional receptor on most nucleated cells of the body. There are 3 major MHC class I genes in HLA: HLA-A, HLA-B, HLA-C and three minor genes HLA-E, HLA-F and HLA-G. 32-microglobulin binds with major and minor gene subunits to produce a heterodimer. MHC molecules of class I consist of a heavy chain and a light chain and can bind a peptide of about 8 to 11 amino acids, but usually 8 or 9 amino acids, if this peptide has suitable binding motifs, and presenting it to cytotoxic T-lymphocytes. The binding of the peptide is stabilized at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow the required flexibility. The peptide bound by the MHC molecules of class I usually originates from an endogenous protein antigen. As an example, the heavy chain of the MHC molecules of class I is typically an HLA-A, HLA-B or HLA-C monomer, and the light chain is β-2-microglobulin, in humans. There are 3 major and 2 minor MHC class II proteins encoded by the HLA. The genes of the class II combine to form heterodimeric (αβ) protein receptors that are typically expressed on the surface of antigen-presenting cells. The peptide bound by the MHC molecules of class II usually originates from an extracellular or exogenous protein antigen. As an example, the α-chain and the β-chain are in particular HLA-DR, HLA-DQ and HLA-DP monomers, in humans. MHC class II molecules are capable of binding a peptide of about 8 to 20 amino acids, notably from 10 to 25 amino acids or from 13 to 25 amino acids if this peptide has suitable binding motifs, and of presenting it to T-helper cells. The peptide lies in an extended conformation along the MHC II peptide-binding groove which (unlike the MHC class I peptide-binding groove) is open at both ends. It is held in place mainly by main-chain atom contacts with conserved residues that line the peptide-binding groove.

The term “peptidome” refers to the complete set of peptides expressed by a particular genome, or present within a particular organism or cell type (such as a cancer cell). Proteomic analysis (proteomics) thus refers to the separation, identification, and quantification of the entire set of peptides or proteins expressed by a genome, a cell, or a tissue at a specific point in time.

Proteomics analysis are typically based on two major techniques, namely two-dimensional gel electrophoresis (2-DGE) (Harper S et al., In: Coligan J E, Dunn B M, Speicher D W, Wing-field P T, editors. Current Protocols in Protein Science. John Wiley & Sons; Hoboken, N.J.: 1998. pp. 10.4.1-10.4.36.) and Mass Spectrometry (MS) (Aebersold & Mann, 2003), which are both powerful methods for the analysis of complex mixtures of proteins. HPLC is an alternative separation technique for proteomic studies, especially in separation and identification of low-molecular-weight proteins and peptides (Garbis et al., 2005). MS allows the determination of the molecular mass of proteins or peptides based on the mass to charge ratio (m/z) of ions in the gas phase. The terms “gel-based” or “gel-free” proteomics are used in relation to the applied separation techniques, 2-DGE or HPLC; proteomics approaches can also be “bottom-up” or “top-down,” which basically identify proteins from their protease (e.g., trypsin) digests or, as a whole, via a mass spectrometer, respectively.

Bottom-up proteomics is a common method to identify proteins from a biological sample (tissue(s) or cells) and characterize their amino acid sequences and post-translational modifications by proteolytic digestion of proteins prior to analysis by mass spectrometry. The crude protein extract is enzymatically digested, followed by one or more dimensions of separation of the peptides typically by liquid chromatography coupled to mass spectrometry, a technique known as shotgun proteomics. By comparing the masses of the proteolytic peptides or their tandem mass spectra with those predicted from a sequence database or annotated peptide spectral in a peptide spectral library, peptides can be identified, and multiple peptide identifications assembled into a protein identification.

In top-down proteomics, intact proteins are purified prior to digestion and/or fragmentation either within the mass spectrometer or by 2D electrophoresis. Top-down proteomics either uses an ion trapping mass spectrometer to store an isolated protein ion for mass measurement and tandem mass spectrometry (MS/MS) analysis or other protein purification methods such as two-dimensional gel electrophoresis in conjunction with MS/MS.

From the data generated by the MS, the protein is either sequenced de novo by manual mass analyses of the spectra or processed automatically via sequence search engines such as SEQUEST, Mascot, Phenyx, X!Tandem, and OMSSA. These algorithms are developed based on the correlation between experimental and theoretical MS/MS data; the latter being generated from in silico digestion of protein databases such as UniProt/Swiss-Prot (Deutsch, Lam, & Aebersold, 2008).

The term “immunopeptidome”, also commonly named “immunopeptidomic pattern”, “pMHC repertoire”, or “MHC-ligandome” or “HLA ligandome”, refers to the complete set of peptides within a particular cell type, which are bound to at least one MHC/HLA molecule at the cell surface. Correspondingly, “immunopeptidomics” has emerged as a term to describe analysis of the MHC/HLA-ligandome. The most common immunopeptidomics methods rely on mass spectrometry (MS). Immunopeptidomics samples are generally prepared by isolating MHCs, for example by using an allele-specific antibody, pan-specific antibody, or engineered affinity tag system, from lysed cells or tissues. Isolated complexes are acid eluted, and peptides are purified from the MHC molecules using molecular weight cut-off filtration (MWCO), solid phase extraction or other techniques, and are subsequently analyzed by MS (see for example for review L. E. Stopfer et al., Immuno-Oncology and Technology, Volume 11, 2021, 100042).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab.

F(ab′)₂, and Fab fragments that lack the Fc fragment of intact antibody, clear more rapidly from the circulation, and may have less non-specific tissue binding of an intact antibody (Wahl et ah, J. Nucl. Med. 24:316-325 (1983).

As used herein, antibodies include whole native antibodies, bispecific antibodies; chimeric antibodies; Fab, Fab′, single chain V region fragments (scFv), fusion polypeptides, and unconventional antibodies.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains (See, e.g., Rabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Rabat system (Rabat, E. A., et al. (1991) Sequences of Proteins of Immunological Interest, Fifth Edition, ET.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay), surface plasmon resonance assays such as BIACORE assays, and kinetic exclusion assays such as KINEXA assays.

The term “chimeric antigen receptor” or “CAR” as used herein refers to a molecule comprising an extracellular antigen-binding domain that is fused to an intracellular signalling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR has a high binding affinity or avidity for the antigen.

The term “antigen-binding domain” as used herein refers to a domain capable of specifically binding a particular antigenic determinant or set of antigenic determinants present on a cell.

The term “immune cell” as herein intended typically encompasses T cells, Natural Killer T cells, CD4+/CD8+ T cells, TILs/tumor derived CD8 T cells, central memory CD8+ T cells, Treg, MAIT, Yδ T cells, human embryonic stem cells, and pluripotent stem cells from which lymphoid cells may be differentiated.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Unless specifically stated or obvious from context, as used herein, the term “about” is to be understood as within a range of normal tolerance in the art, for example within 2 standard deviations of the mean. About can be understood as within 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### Method for Selecting a Tumor Neoantigenic Peptide

The method for selecting a tumor neoantigenic peptide as per the present disclosure comprises
a step of identifying, among mRNA sequences from a cancer cell sample of a subject, a fusion transcript (JET) sequence comprising a transposable element (TE) sequence and an exonic sequence, and including an open reading frame (ORF), and
a step of selecting a tumor neoantigenic peptide of at least 8 amino acids, encoded by a part of said ORF of the fusion transcript sequence,
   wherein said ORF overlaps the junction between the TE and the exonic sequence, is pure TE and/or is non-canonical, and
   wherein said tumor neoantigenic peptide binds to at least one Major Histocompatibility Complex (MHC) molecule of said subject.

Typically, a peptide translated from a part of non-canonical ORF of an exonic sequence is recognized as non-self by the immune system.

In some embodiments, the exonic sequence is from an oncogene and/or a tumor suppressor gene or one of their mutated variants.

Conceptually, cancer is a result of consecutive somatic mutation accumulation. Many studies have shown that both the gain of function in oncogenes and the loss of function in tumor-suppressor genes are required for the development of cancer from a normal cell. For a diploid organism, gain-of-function mutations are often dominant or semi-dominant, whereas loss-of-function mutations are usually recessive. Two-hit hypothesis of oncogenesis proposes that the development of cancer is initiated by the loss of both alleles of a tumor-suppressor gene.

Oncogenes (also named cancer genes) are genes whose action positively promotes cell proliferation or growth. The normal nonmutant versions are known as proto-oncogenes. The mutant versions are excessively or inappropriately active leading to tumor growth. Oncogenes can be identified in the Cancer Gene Marker Database (CGMD) (Pradeepkiran, J., Sainath, S., Kramthi Kumar, K. et al. CGMD:. Sci Rep 5, 12035 (2015) “An integrated database of cancer genes and markers”). Oncogenes (ONCs) can also be downloaded from Network of Cancer Genes database (NCG 5.0) (An O, Dall'Olio GM, Mourikis T P, Ciccarelli F D, Nucleic Acids Res. 2016 Jan. 4; 44 (D1): D992-9; “NCG 5.0: updates of a manually curated repository of cancer genes and associated properties from cancer mutational screenings”). Non-limitatives examples of oncogenes include: L-MYC, LYL-1, LYT-10, LYT-10/Cal, MAS, MDM-2, MLL, MOS, MTG8/AML1, MYB, MYH11/CBFB, NEU, N-MYC, OST, PAX-5, PBX1/E2A, PIM-1, PRAD-1, RAF, RAR/PML, RAS-H, RAS-K, RAS-N, REL/NRG, RET, RHOM1, RHOM2, ROS, SKI, SIS, SET/CAN, SRC, TAL1, TAL2, TAN-1, TIAM1, TSC2, and TRK.

Tumor suppressor genes (also named anti-oncogenes) represent the opposite side of cell growth control, normally acting to inhibit cell proliferation and tumor development. Thus tumor suppressor genes are genes that normally suppress cell division or growth. Loss of TSG function promotes uncontrolled cell division and tumor growth. Rb, a tumor suppressor gene that was identified by the genetic analysis of retinoblastoma an encoding atranscriptional regulatory protein, served as the prototype for the identification of additional tumor suppressor genes that contribute to the development of many different human cancers. Tumor suppressor genes are notably described in “Cooper GM. The Cell: A Molecular Approach. 2nd edition. Sunderland (MA): Sinauer Associates; 2000. Tumor Suppressor Genes”. Tumor-suppressor genes (TSGs) can also be downloaded from Tumor Suppressor Gene database (TSGene 2.0) (see for reference Zhao M, Kim P, Mitra R, Zhao J, Zhao Z; Nucleic Acids Res. 2016 Jan. 4; 44 (D1): D1023-31; “TSGene 2.0: an updated literature-based knowledgebase for tumor suppressor genes”). In this context, non-limitative examples of tumor suppressor genes include: APC, BRCA1, BRCA2, DPC4, INK4, MADR2, NF1, NF2, p53, PTC, PTEN, Rb, RB1, VHL, WT1, BUB1, BUBR1, TGF-βRII, Axin, DPC4, p300, PPARγ, p16, DPC4, PTEN, and hSNF5.

Oncogenes, tumor suppressor genes or “double agent” genes (with both oncogenic and tumor-suppressor functions) can be systematically identified through database search and text mining. Indeed, information on oncogenes or tumor suppressor genes can typically be found in Ensembl database (but see also Shen L, Shi Q, Wang W. Double agents: genes with both oncogenic and tumor-suppressor functions. Oncogenesis. 2018; 7 (3): 25. Published 2018 Mar. 13). Double agent genes may be identified as genes overlapped between the two above mentioned databases (see also Shen et al., Oncogenesis 2018 above).

Without to be bound by any theory, the inventors believe that selection of fusion wherein the exonic sequence is from an oncogene and/or a tumor suppressor gene is of high relevance for the reason below:

TE insertion in oncogenes can alter their oncogenic activity. Insertion of TE sequences in oncogene active domains could therefore result in constitutive activity of the oncogenes, similar to driver mutations. These fusions giving chimeric oncogenes could thus represent a new family of oncogenic proteins. If this is the case, targeting the activity of these new “fusion oncogenes” with small molecule antagonists could represent a potential therapeutic approach for cancer where these chimeric oncogenes are expressed.

TE insertions in tumor suppressors could inactivate their suppressor functions, leading typically to a loss of function (for example through introduction of stop codons, changes in ORF or disruptive amino acid stretches), thereby contributing to the oncogenic process.

Fusions implicating cancer driver genes would be excellent targets for adoptive cell therapies, antibodies, ADCs, T cell engagers, etc. If they are involved in oncogenesis, fusions oncogenes are expected to be more specific for cancer cells, and thus to reduce the development of resistances (because of the oncogenic activity of the target).

In some embodiments, the TE sequence is located in 5′ end of the fusion transcript sequence (it is also said that the TE sequence is the donor sequence) and the exonic sequence is located in 3′ end of the fusion transcript sequence with respect to the junction (the exon sequence is thus called an acceptor sequence). The expression “is located in 5′ end of the fusion transcript sequence” means that the element is located upstream of the junction in the fusion transcript sequence. The expression “is located in 3′ end of the fusion transcript sequence” means that the element is located downstream of the junction in the fusion transcript sequence.

In a particular embodiment, the TE sequence is located in 5′ end of the fusion transcript (JET) sequence and the exonic sequence is located in 3′ end of the fusion transcript (JET) sequence, and the part of the ORF of said fusion transcript (JET) sequence, which encodes the neoantigenic peptide (pJET), overlaps the junction. In this case, the ORF can be canonical or non-canonical. It is understood that the ORF may comprise the junction but the neoantigenic peptide (pJET) may not derive from the junction. In some embodiments however, where the neoantigenic peptide has a sequence which is derived from the junction sequence, the obtained peptide is thus encoded by both TE sequence and exonic sequence.

The expression “the part of the ORF is overlapping or overlaps the junction between the TE sequence and the exonic sequence”, means that said junction is contained in the part of the ORF of the fusion transcript (JET) sequence, which encodes said neoantigenic peptide (pJET).

In embodiments wherein (i) the part of the ORF encoding the neoantigenic peptide is overlapping the junction between the TE sequence and the exonic sequence, and (ii) the TE sequence and the exonic sequence are respectively in 5′ end and 3′end of the fusion transcript (JET) sequence, said part of the ORF typically encodes a neoantigenic peptide of at least 8 amino acids, including at least between 1 to 6 amino acids, notably 2 to 6 from the TE sequence and at least between 1 and 6, notably 2 to 6 amino acids from the exonic sequence.

In another embodiment wherein the TE sequence is located in 5′ end of the fusion transcript (JET) sequence and the exonic sequence is located in 3′ end of the fusion transcript (JET) sequence, the part of ORF which encodes said neoantigenic peptide, is downstream of the junction and the ORF is thus non-canonical.

The expression “the part of the ORF is downstream of the junction” means that the part of the ORF encoding the neoantigenic peptide (pJET) is not overlapping the junction, but it is contained in the 3′end part of said fusion transcript sequence with respect to the junction. In this embodiment, as the 3′ end part with respect to the junction, is the exonic sequence, the part of the ORF encoding the neoantigenic peptide is thus contained in the exonic sequence. Thus, as the part of the ORF is only located in the exonic sequence, the obtained peptide is therefore encoded by the exonic sequence, in a non-canonical ORF. Thus, in the particular embodiment wherein the exonic sequence is located in 3′ end of the fusion transcript (JET) sequence with respect to the junction, and wherein the part of the ORF which encodes the neoantigenic peptide is downstream of the junction with a non-canonical reading frame, the part of the ORF of the fusion transcript (JET) sequence encodes a neoantigenic peptide including 0 amino acid from the TE sequence, and at least 8 amino acids from the exonic sequence.

In another embodiment, the TE sequence is located in 3′ end of the fusion transcript (JET) sequence and the exonic sequence is located in 5′ end of the fusion transcript sequence with respect to the junction.

In some embodiments, the TE sequence is located in 3′ end of the fusion transcript (JET) sequence and the exonic sequence is located in 5′ end of the fusion transcript sequence and the part of the ORF of said fusion transcript sequence, which encodes a neoantigenic peptide, is overlapping the junction between the TE sequence and the exonic sequence. In this case, the ORF can also be canonical or non-canonical. The obtained peptide (pJET) is encoded by both TE sequence and exonic sequence.

In the particular embodiment wherein the part of the ORF encoding the neoantigenic peptide (pJET), is overlapping the junction between the exonic sequence and the TE sequence, and wherein the exonic sequence and the TE sequence are respectively in 5′ end and 3′end of the fusion transcript (JET) sequence, said part of the ORF encodes a neoantigenic peptide (pJET) of at least 8 amino acids, including at least between 1 to 6, notably 2 to 6 amino acids from the TE sequence and at least between 1 and 6, notably 2 to 6 amino acids from the exonic sequence.

In still another embodiment, the TE sequence is located in 3′ end of the fusion transcript (JET) sequence, the exonic sequence is located in 5′ end of the fusion transcript sequence, and the part of the ORF which encodes a neoantigenic peptide (pJET), is downstream of the junction between the exonic sequence and the TE sequence. Optionally, the peptide sequence (pJET) which is thus encoded by the pure TE sequence is non-canonical.

In this embodiment, as the 3′ end part with respect to the junction is the TE sequence, the part of the ORF encoding the neoantigenic peptide is therefore encoded by the TE sequence. Thus, the part of the ORF encodes a neoantigenic peptide including no amino acid from the exonic sequence and at least 8 amino acids from the TE sequence. In the particular embodiment wherein the TE sequence is located in 3′ end of the fusion transcript sequence with respect to the junction, and the part of the ORF which encodes the neoantigenic peptide is downstream the junction, the part of the ORF of the fusion transcript (JET) sequence encodes a neoantigenic peptide (pJET) including 0 amino acid from the exonic sequence, and at least 8 amino acids from the TE sequence.

A tumor neoantigenic peptide is a peptide that arises from somatic alterations (classically mutations in the DNA sequence), is recognized as different from self, and is presented by antigen-presenting cells (APC), such as dendritic cells (DC) and tumor cells themselves. Cross-presentation plays an important role as the APC is able to translocate exogenous antigens from the phagosome into the cytosol for proteolytic cleavage into the major histocompatibility complex I (MHC I) epitopes by the proteasome.

In the present disclosure the alteration corresponds to the transcription of fusion mRNA sequences that comprise a transposable element (TE) sequence and an exonic sequence (JET). This may arise from somatic (i.e.: specifically in the tumor clone) transposition. It may also arise not from de novo transposition but from tumor specific transcriptional de-repression such that a TE and nearby gene are co-transcribed.

A neoantigenic peptide (pJET) according to the present disclosure may be completely absent from normal healthy samples (i.e., not expressed in normal healthy samples) and thus be specific to tumor samples. Alternatively, it may be expressed at low levels in normal cells and/or disproportionately expressed on tumor samples as compared to normal (healthy) samples.

It can also be selectively expressed by the cell lineage from which the cancer evolved.

Cancer or tumor samples according to the present disclosure can be isolated from any solid tumor or non-solid tumor of any of the tissues or organs as defined previously, for example, breast cancer, lung cancer and/or melanoma. In some embodiments cancer samples are from Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma or Uveal Melanoma samples. In a particular embodiment, cancer samples are from lung cancer samples, notably from LUAD samples.

Typically, as per the present disclosure, the step of identifying the fusion transcript sequences is carried out by mapping mRNA sequences from cancer sample against a reference genome, and then distinguishing normal and abnormal (non-canonical) junctions.

According to the present disclosure, normal junctions correspond to junctions between donor and acceptor on the same strand and not too far apart (e.g.: typically not on different chromosomes).

According to the present disclosure, abnormal junctions correspond to junctions between donor and acceptor sequences on different chromosomes, or in cis (same chromosomes) but on different strands (no matter the order and the 5′-3′ sense).

mRNA sequences typically usable according to the present disclosure are RNA seq data (as illustrated in the results herein). RNA seq data are typically obtained from purified RNA obtained from a cell or a tissue sample, fragmented and reverse-transcribed into cDNA. The obtained cDNA are then amplified and sequenced (next-generation sequencing-NGS) on a high-throughput platform (such as for example the Illumina GA/HiSeq-see http://www.illumina.com—, SOLID or Roche 454). This process generates millions of short reads taken from one end of the cDNA fragments. A common variant on this process is to generate reads from both ends of each cDNA fragment, known as “paired-end” reads.

In some embodiments, the mRNA sequences can be mapped against a corresponding reference genome or transcriptome (such as the human reference genome Hg19 ENSEMBL (RNA sequences, GRCh37) with an adapted software, such as for example: Spliced Transcripts Alignment to a Reference (i.e.: STAR-see Dobin, Alexander et al. “STAR: ultrafast universal RNA-seq aligner.” Bioinformatics (Oxford, England) vol. 29, 1 (2013): 15-21), TopHat2 (Kim, Daehwan et al. “TopHat2: accurate alignment of transcriptomes in the presence of insertions, deletions and gene fusions.” Genome biology vol. 14, 4 R36. 25 Apr. 2013, doi: 10.1186/gb-2013-14-4-r36) or HISAT (Kim, Daehwan et al. “HISAT: a fast spliced aligner with low memory requirements.” Nature methods vol. 12, 4 (2015): 357-60. doi: 10.1038/nmeth.3317). STAR is a standalone software that uses sequential maximum mappable seed search followed by seed clustering and stitching to align RNA-seq reads. It can typically detect canonical junctions, non-canonical splices, and fusion/chimeric transcripts. Typically, detection of the junctions can be performed as detailed in the results based on the definitions from ENSEMBL and RepeatMasker databases respectively, downloaded from the UCSC Genome Browser. Thus, in some embodiments, the normal and abnormal junctions are determined in silico using dedicated databases, such as for example Ensembl and Repeatmasker databases, and the fusion transcripts having junctions between a TE and an exonic sequence are extracted in silico.

More particularly, in some embodiments, RNAseq reads from a sample (or cell) of interest are aligned to a reference genome (such as typically the hg19 genome) using typically STAR two-pass mode27 to identify un-annotated junctions. As previously indicated JETs are identified as a junction between an exon (most particularly a coding DNA sequence-CDS-exon) and a TE (or repeated element, RE). As per the present disclosure TE (or RE) can be identified (i.e. filtered) according to the definition of commonly used databases in the field such as ENSEMBL (GRCh37) and RepeatMasker.

According to the present disclosure, the mRNA sequences can come from all types of cancer cell or tumor cell sample(s). The tumor may be a solid or a non-solid tumor. In particular, the mRNA sequences come from any tissues or organs affected by a cancer or tumor as previously defined, for example from breast cancer, lung cancer and/or melanoma. In a particular embodiment, mRNA sequences are from LUAD samples. Tumor samples can be for example obtained from the Cancer Genome Atlas (TCGA). In some embodiments, the mRNA sequences are obtained from cell lines such as for example tumor cell lines from the Cancer Cell Line Encyclopedia (CCLE).

In some embodiments, the number of splicing reads can be normalized by the number of unique mapped reads. Typically JETs with a level of expression over 2.10⁻⁷ are selected.

In some embodiments of the present disclosure, the fusion transcript sequences are shared in more than 1%; notably more than 5%, more than 10%, more than 15%, more than 20% or even more than 25% of cancer samples (typically obtained from various patients, for example from the cancer samples collected for a given cancer type in the TCGA) and/or cell lines. In some embodiments, a fusion transcript (JET) sequence as per the present disclosure is shared in cancer samples from more than 1%; notably more than 2%, more than 5%, more than 10%, more than 15%, more than 20% or even more than 25% of the subjects suffering from a cancer. Alternatively, or additionally, the fusion (JET) transcript sequence may be specific for a cancer type of shared between several cancer types. Alternatively, or in addition, a fusion transcript sequence can be expressed in at least 1; 2; 3; 4; 5; 6; 7; 8; 9; 10; 11; 12; 13; 14; 15; 16; 17; 18; 19; 20 cell lines (typically from the CCLE).

According to the present disclosure, the fusion (JET) transcript sequences are expressed at higher levels in tumor cells compared to normal healthy cells. In some embodiments, the fusion transcript sequence is expressed in cancer cells (obtained from one or more cancer samples and/or one or more cell lines) and not in healthy cells (obtained from one or more tissue samples or one or more cell lines), in particular not in thymus healthy cells. In some embodiments a JET is considered not expressed in a cell when its expression level is below 2.10-7, notably below 2.10-8 and typically not detectable. Such fusion (JET) transcript may be called tumor specific fusion (JET) as per the present disclosure. Fusion transcripts that are expressed at higher level(s) in tumor cells as compared to normal cell, typically that are disproportionally expressed in cancers cells as compared to normal cells as defined above may be called tumor associated fusion transcripts (TAF) as per the present disclosure. Tumor associated fusion transcripts may be selected according to the present application if they are present in more than 10% of the tumor samples (for example in more than 1, notably more than 2%, more than 5% and in particular more than 10% of tumor samples obtained from the TCGA database for the same cancer type) and in less than 20% of the normal samples (for example juxta-tumor samples from the TCGA). Alternatively, or in addition, a fusion transcript sequence can be expressed in at least 1; 2; 3; 4; 5; 6; 7; 8; 9; 10; 11; 12; 13; 14; 15; 16; 17; 18; 19; 20 cell lines.

In some embodiments, the method further comprises a step of determining, optionally in silico or using in vitro techniques (see notably the examples for illustration), the binding affinity of the tumor neoantigenic peptide with at least one MHC molecule of the said subject suffering from a cancer.

When the method is carried out on human samples, the method may comprise a step of determining the patient's class I or class I Major Histocompatibility Complex (MHC, aka human leukocyte antigen (HLA) alleles). It is to be noticed that as MHC alleles for laboratory mice are generally known such that this step may not be necessary in that particular context. In the present application, “MHC molecule” refers to at least one MHC class I molecule or at least one MHC Class II molecule.

An MHC allele database is carried out by analyzing known sequences of MHC I and MHC II and determining allelic variability for each domain. This can be typically determined in silico using appropriate software algorithms well-known in the field. Several tools have been developed to obtain HLA allele information from genome-wide sequencing data (whole-exome, whole-genome, and RNA sequencing data), including OptiType, Polysolver, PHLAT, HLAreporter, HLAforest, HLAminer, and seq2HLA (see Kiyotani K et al., Immunopharmacogenomics towards personalized cancer immunotherapy targeting neoantigens; Cancer Science 2018; 109:542-549). For example, the seq2hla tool (see Boegel S, Lower M, Schafer M, et al. HLA typing from RNA-Seq sequence reads. Genome Med. 2012; 4:102), which is well designed to perform the method as herein disclosed is an in silico method written in python and R, which takes standard RNA-Seq sequence reads in fastq format as input, uses a bowtie index (Langmead B, et al., Ultrafast and memory-efficient alignment of short DNA sequences to the human genome. Genome Biol. 2009, 10: R25-10.1186/gb-2009-10⁻³-r25) comprising all HLA alleles and outputs the most likely HLA class I and class II genotypes (in 4 digit resolution), a p-value for each call, and the expression of each class.

Typically, the sequences having junctions between a TE and an exonic sequence (JETs) are extracted in silico. The affinity of all possible peptides encoded by each sequence for each MHC allele from the patient (or mouse) can be for example determined in silico using computational methods to predict peptide binding-affinity to HLA molecules. Indeed, accurate prediction approaches are based on artificial neural networks with predicted IC₅₀. For example, NetMHCpan software which has been modified from NetMHC to predict peptides binding to alleles for which no ligands have been reported, is well appropriate to implement the method as herein disclosed (Lundegaard C et al., NetMHC-3.0: accurate web accessible predictions of human, mouse and monkey MHC class I affinities for peptides of length 8-11; Nucleic Acids Res. 2008; 36: W509-W512; Nielsen M et al. NetMHCpan, a method for quantitative predictions of peptide binding to any HLA-A and -B locus protein of known sequence. PLOS One. 2007; 2: e796, but see also Kiyotani K et al., Immunopharmacogenomics towards personalized cancerimmunotherapy targeting neoantigens; Cancer Science 2018; 109:542-549 and Yarchoan M et al., Nat rev. cancer 2017; 17 (4): 209-222). NetMHCpan software predicts binding of peptides to any MHC molecule of known sequence using artificial neural networks (ANNs). The method is trained on a combination of more than 180,000 quantitative binding data and MS derived MHC eluted ligands. The binding affinity data covers 172 MHC molecules from human (HLA-A, B, C, E), mouse (H-2), cattle (BoLA), primates (Patr, Mamu, Gogo) and swine (SLA). The MS eluted ligand data covers 55 HLA and mouse alleles.

In example embodiments, neoantigenic peptides encoded by fusion (JETs) transcripts as above described and having a Kd affinity for MHC alleles of less than 10⁻⁴, 10⁻⁵, 10⁻⁶, 10⁻⁷ M or less than 500 nM, notably less than 50 nM are selected as tumor neoantigenic peptides.

As above mentioned, affinity of the selected peptide for MHC alleles can be determined in silico using appropriate software such as netMHCpan. Thus, in some embodiments, neoantigenic peptides bind MHC class I with a binding affinity of less than 2% percentile rank score predicted by NetMHCpan 4.0. In other embodiments, the neoantigenic peptides bind MHC class II with a binding affinity of less than 10% percentile rank score predicted by NetMHCpanII 3.2.

Affinity can also (alternatively or in addition) be estimated in vitro, for example by using MHC tetramer formation assay as described in the results included therein (see example 2, point 2.1 and 2.2.2). Commercial assays for example from ImmunAware® can typically be used by the skilled person (EasYmers® kits are from ImmunAware® are notably used according to their training guide). Typically, binding affinity is determined as a percentage of binding to a positive control. Generally, peptides showing a percentage of binding of at least 30%, notably at least 40% or even at least 50% of the positive control are selected. Typically, the neoantigenic peptide as per the present disclosure, and typically obtainable as per the present method, binds at least one HLA/MHC molecule with an affinity sufficient for the peptide to be presented on the surface of a cell as an antigen. Generally, the neoantigenic peptide has an IC50 affinity of less than 10⁻⁴, or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less for at least one HLA/MHC molecule (lower numbers indicating greater binding affinity), typically a molecule of said subject suffering from a cancer.

Further optional steps according to the present method may thus independently include:
a step of exclusion of fusion transcripts or predicted peptides expressed at high levels or high frequency on healthy cells. An alignment of the fusion transcript sequence against the RNAseq data of healthy cells, typically allows determining the relative amount of fusion transcript sequence(s) present in healthy cells; In one embodiment, fusion transcripts or predicted peptides expressed on healthy cells are discarded.
a step to confirm that a tumor neoantigenic peptide is not expressed in healthy cells of the subject. This step can be carried out using typically the Basic local alignment search tool (BLAST) and performing alignment of the sequence of the neoantigenic peptide against the proteome of healthy cells; Preferably, peptides that align against the proteome of normal healthy cells (for example using BLAST) are discarded.
a step to confirm that the fusion transcript or predicted peptide is expressed in cancer cells of the subject. The presence of the selected fusion transcript sequence in cancer cells can be checked typically by RT-PCR in mRNA extracted from cancer cell sample.

In some embodiments, the present method can also include a step wherein the identified fusion (JETs) transcripts are in silico translated to generate a JET-derived protein database (JET-db). Typically, Strand-indexed JETs containing gene as donor can be translated using the canonical ORF from the implicated gene until the first stop codon after the breakpoint. In JETs where TE was the donor, the 3 possible ORFs can be translated and only the sequence found more proximal to the breakpoint and between two stop codons is typically kept. This JET db (typically also concatenated to the human proteome) can be then interrogated in mass spectrometry based proteomic datasets obtained from tumor samples and/or tumor cell lines which typically consist in proteomics data obtained from tumors samples and/or tumor cell lines. In some embodiments, public mass spectrometry datasets can be used. This embodiment is notably well described in the results provided in the present application. Such analysis also to identify JET-derived peptides or proteins.

In more specific embodiments, the JETdb (typically concatenated to the human proteome) can be interrogated to immunonopeptidomics mass spectrometry-based datasets as also detailed in the examples included herein. This embodiment allows to identify JET-derived peptides or proteins (pJETs) that are presented to MHC molecules.

To ensure that JET-derived peptides did not match with canonical proteins or peptides derived from JETs found in normal samples, identified peptides can be filtered for example with UniProt/TrEMBL database and/or with in silico translated JETs from normal (including for example juxta-tumor) samples or cell(s) (for example from public databases such as the TCGA or the CCLE).

### Neoantigenic Peptides

The present disclosure also relates to an isolated tumor neoantigenic peptide comprising at least 8, 9, 10, 11, or 12 amino acids, encoded by a portion of an open reading frame (ORF) from a fusion transcript that is a human mRNA sequence comprising a transposable element (TE) sequence and an exonic sequence. The peptide may be 8-9, 8-10, 8-11, 12-25, 13-25, 12-20, or 13-20 amino acids in length. Although the ORF overlaps a junction between a TE sequence and an exonic sequence, it is understood that the tumor neoantigenic peptide itself may not comprise the junction.

More particularly, the present disclosure provides a selection of isolated neoantigenic tumor peptide candidates obtained from the fusion transcripts predicted from the bioinformatic tumor transcriptome database CCLE (Broad Institute Cancer Cell Line Encyclopedia).

This step has been detailed in the above section (detailed description and previous examples) of the present application. Briefly, and as previously mentioned, in these fusion transcripts, the TE can be donor (in 5′ position) or acceptor (in 3′ acceptor) and correspondingly the exon can be acceptor or donor. TE-exon splicing results in the incorporation of parts of the “non-coding” genome into the coding genome, thereby exposing non-coding genomic sequences to the translation machinery. These fusions (or chimeric) transcripts also named JET (Junction Exon TE) include an ORF (open reading frame). When the TE is acceptor, the ORF of the fusion transcript is canonical (i.e. the same as the canonical transcript), whereas when the TE is the donor the ORF can be canonical (generally ORF1) or can be shifted by 1 or 2 nucleotides (typically ORFs 2 and 3 respectively). The fusion transcripts include not only the fused TE and exon sequences but can also further include exon(s), upstream the fusion breakpoint (between the exon and the TE) if the exon is donor or downstream the fusion breakpoint is the TE is donor, corresponding to the various transcript isoforms. Amino acid sequences SEQ ID NO:1-38907 and 38916-41099 correspond to the in silico translated sequences of all the transcripts (splice variants) including a fusion event (a fusion between an exonic sequence and TE sequence also named herein JET for “Junction Exon TE”), which are thus also named “fusion transcripts” or “chimeric transcripts”. Amino acid sequences are also named translated fusion transcripts or shortly “translated fusions” or “translated JETs” or “pJETs. Sequences SEQ ID NO: 1-10170; 38760-38907; 38916-39683; 39924-39965; 40128-40193; 40365-40391; and 40510-40728 are translated amino acid sequences from exon donor derived fusion transcripts. Sequences SEQ ID NO:10171-38759; 38760-38907; 38916-39683; 39684-39923; 399666-40127; 40194-40364; 40392-40509; and 40729-41099 are translated amino acid sequences from TE donor derived fusion transcripts. Tables 9; 11; 13; 15; 17 and 19 and 10; 12; 14; 16; 18; 20 herein provides the reference, location and coordinate of the donor sequence (exon or TE) and acceptor sequence (TE or exon respectively) such that each fusion transcript sequence can be unambiguously retrieved. Tables 9; 11; 13; 15; 17 and 19 and 10; 12; 14; 16; 18; 20 also assign each transcript to the corresponding translated fusion transcripts identified as SEQ ID NO: 1-38907 and 38916-41099. For each translated fusion transcript, the position of the breakpoint (between the exonic and TE sequences) is provided such that it is possible to identify for each of the amino acid sequences of SEQ ID NO:1-38907 and 38916-41099, the TE-derived sequence and the exon-derived sequence.

Thus, the present disclosure encompasses isolated tumor neoantigenic peptides, optionally comprising at least 8 amino acids, wherein said neoantigenic peptides are encoded by a part of an open reading frame (ORF) from a fusion transcript encoding any one of SEQ ID NO:1-38907 and 38916-41099 and comprising a transposable element (TE) sequence and an exonic sequence, wherein said ORF overlaps the junction between the TE and the exonic sequence, is pure TE and/or is non-canonical.

In some embodiments, the present disclosure encompasses isolated tumor neoantigenic peptide, optionally of at least 8 amino acids, wherein the neoantigenic peptide:
is a part of any one of the sequences of SEQ ID NO:1-38759; 39587-41089, notably is of any one of SEQ ID NO:38760-38907 and 38916-39596 or a fragment thereof, and comprises a TE-derived amino acid sequence, or
is a part of any one of the sequence of SEQ ID NO: 1-10170; 39597-39683; 39924-39965; 40128-40193; 40365-40311; and 40510-40728, notably is of any one of SEQ ID NO: 38760-38907 and 38916-39596 or a fragment thereof and is encoded by a fusion wherein the exon is the donor. Typically said neoantigenic peptides are derived from a part of a non-canonical ORF of a fusion transcript as above defined.

In some embodiments, the neoantigenic peptide overlaps the breakpoint between the TE-derived sequence and the exon-derived sequence of the translated fusion transcripts of any one of SEQ ID NO: 1-38759; 39597-41099, including fragments thereof. In other embodiments the neoantigenic peptide consists in or comprises a pure TE-derived sequence from a translated fusion transcripts of any one of SEQ ID NO:1-38759; 39597-41099, including fragments thereof.

in some embodiments the neoantigenic peptide comprises at least 8, 9, 10, 11 or 12 amino acids.

The peptide may be 8-9, 8-10, 8-11, 12-25, 13-25, 12-20, or 13-20 amino acids in length and fulfills one or more of the neoantigen peptide characteristics described above. The N-terminus of the peptide of at least 8 amino acids may be encoded by the triplet codon starting at any of nucleotide positions 1, 2, 3, 4, 5, 6, 7, 8, 9, 10, 11, 12, 13, 14, 15, 16, 17, 18, and higher (it being understood that the disclosure contemplates a start position that is any of the integers between 1 and 8000 without having to list every number between 1 and 8000). More specifically, the N terminus of the neoantigenic peptide of at least 8 amino acids may start at any of amino acid positions 1, 2, 3, 4, 5, 6, 7, 8, 9, 10, 11, 12, 13, 14, 15, 16, 17, 18, and higher (it being understood that the disclosure contemplates a start position that is any of the integers between 1 and 15000 without having to list every number between 1 and 15000) of any one of the sequences SEQ ID NO: 1-38759. Typically however, and as mentioned above, the neoantigenic peptide:
is a fragment of any one of the sequence of SEQ ID NO:1-38759; 39597-41099 and comprises a TE-derived amino acid sequence, or
is a fragment of any one of the sequence of SEQ ID NO: 1-38759; 39597-41099. Typically said neoantigenic peptides are derived from a part of a non-canonical ORF of a fusion transcript as above defined.

In some embodiments, the neoantigenic peptide overlaps the breakpoint between the TE-derived sequence and the exon-derived sequence of the translated fusion transcripts of any one of SEQ ID NO:1-1-38759; 39597-41099. In other embodiments the neoantigenic peptide consists in or comprises a pure TE-derived sequence from a translated fusion transcripts of any one of SEQ ID NO: 1-38759; 39597-41099.

A peptide as above defined is typically obtainable according to the method of the present disclosure and thus encompasses one or more of the characteristics as previously described. In particular a neoantigenic peptide as per the present disclosure may exhibit one or a combination of the following further characteristics:
It binds or specifically binds MHC class I of a subject and is 8 to 11 amino acids, notably 8, 9, 10, or 11 amino acids. Typically the neoantigenic peptide is 8 or 9 amino acids long, and binds to at least one MHC class I molecule of the subject; or alternatively, it binds to at least one MHC class II molecule of said subject and contains from 12 to 25 amino acids, notably is 12, 13, 14, 15, 16, 17, 18, 19, 20, 21, 22, 23, 24 or 25 amino acids long.
It binds at least one HLA/MHC molecule of said subject suffering from a cancer with an affinity sufficient for the peptide to be presented on the surface of a cell as an antigen. Typically the neoantigenic peptide has an IC50 of less than 10⁻⁴, or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less (lower numbers indicating greater binding affinity).
It does not induce a significant autoimmune response and/or invoke immunological tolerance when administered to a subject.
It is expressed at higher levels in tumor samples compared to normal healthy samples. Typically, as per the present disclosure, a fusion transcript may be selected if it is present in more than 1%, notably more than 2%, more than 5% and typically more than 10% of the tumor samples (from the same or different tumor type, typically from one or more subjects typically from TCGA tumor samples) and in less than 20%, notably less than 15%, less than 10%, less than 5%, less than 2% or even less than 1% of the normal samples. Alternatively, or in addition, the transcript can be identified in one or more (at least 2, 5, 10, 20, 50, 100 cell lines such as for example from the CCLE) In some embodiments, the neoantigenic is more specifically a tumor specific antigen (TSA), i.e.: it is only expressed in cancer sample and not in normal samples, or is expressed at relatively low levels in normal samples (e.g. the expressed mRNA sequences represent minor species in normal cells from normal samples).
It comprises the junction between the TE sequence and the exonic sequence, in other words it is encoded by a part of a TE sequence and a part of an exonic sequence, the ORF being either canonical or non-canonical or
It is encoded by a non-canonical ORF of an exonic sequence or
It is encoded by the TE sequence, optionally in a non-canonical ORF

A tumor neoantigenic peptide may first be validated by RT transcription analysis of fusion transcripts sequence in tumors cell from a subject. Typically also, immunization with a tumor neoantigenic peptide as per the present disclosure elicits a T cell response

Affinity for MHC alleles can be determined by known techniques in the field and notably in silico or in vitro as exemplified above;

In a particular embodiment, a tumor neoantigenic peptide as per the present disclosure binds to a MHC molecule present in at least 1%, 5%, 10%, 15%, 20%, 25% or more of subjects. Notably, a tumor neoantigenic peptide as herein disclosed is expressed in at least 1%, 5%, 10%, 15%, 20%, 25% of subjects from a population of subjects suffering from cancer

More particularly, a tumor neoantigenic peptide of the present disclosure is capable of eliciting an immune response against a tumor present in at least 1%, 5%, 10%, 15%, 20%, or 25% of the subjects in the population of subjects suffering from cancer.

As previously defined, cancer may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus. For example, the tumors or cancers as per the present application includes leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. Examples thereof are lung carcinomas, mamma carcinomas, prostate carcinomas, colon carcinomas, renal cell carcinomas, cervical carcinomas, or metastases of the cancer types or tumors described above. The term cancer according to the present disclosure also comprises cancer metastases and relapse of cancer.

Typically, a neoantigenic peptide as per the present disclosure does not induce a significant autoimmune response and/or invoke immunological tolerance when administered to a subject. Tolerating mechanisms involve clonal deletion, ignorance, anergy, or suppression in the host w the reduction in the number of high-affinity self-reactive T cells.

The neoantigenic peptide can also be modified by extending or decreasing the compound's amino acid sequence, e.g., by the addition or deletion of amino acids. The peptides can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions are generally made in accordance with the following Table 1 when it is desired to finely modulate the characteristics of the peptide.

TABLE 1

| Original residue | Exemplary substitution |
|---|---|
| Ala | Ser |
| Arg | Lys, His |
| Asn | Gln |
| Asp | Glu |
| Cys | Ser |
| Gln | Asn |
| Glu | Asp |
| Gly | Pro |
| His | Lys, Arg |
| Ile | Leu, Val |
| Leu | Ile, Val |
| Lys | Tyr, Trp |
| Met | Thr |
| Phe | Ser |
| Ser | Tyr, Phe |
| Tyr | Trp, Phe |
| Val | Ile, Leu |
| Pro | Gly |

Substantial changes in function (e.g., affinity for MHC molecules or T cell receptors) are made by selecting substitutions that are less conservative than those in above Table, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) hydrophilic residue, e.g. seryl, is substituted for (or by) a hydrophobic residue, e.g. leucyl, isoleucyl, phenylalanyl, valyl or alanyl; (b) a residue having an electropositive side chain, e.g., lysl, arginyl, or histidyl, is substituted for (or by) an electronegative residue, e.g. glutamyl or aspartyl; or (c) a residue having a bulky side chain, e.g. phenylalanine, is substituted for (or by) one not having a side chain, e.g., glycine.

The peptides and polypeptides may also comprise isosteres of two or more residues in the neoantigenic peptide or polypepeptides. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

In addition, the neoantigenic peptide may be conjugated to a carrier protein, a ligand, or an antibody. Half-life of the peptide may be improved by PEGylation, glycosylation, polysialylation, HESylation, recombinant PEG mimetics, Fc fusion, albumin fusion, nanoparticle attachment, nanoparticulate encapsulation, cholesterol fusion, iron fusion, or acylation.

Modifications of peptides and polypeptides with various amino acid mimetics or unnatural amino acids are particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half life of the peptides of the present disclosure is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides and polypeptides may be modified to provide desired attributes other than improved serum half-life. For instance, the ability of the peptides to induce CTL activity can be enhanced by linkage to a sequence which contains at least one epitope that is capable of inducing a T helper cell response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the peptide may be linked to the T helper peptide without a spacer.

The neoantigenic peptide may be linked to the T helper peptide either directly or via a spacer either at the amino or carboxy terminus of the peptide. The amino terminus of either the neoantigenic peptide or the T helper peptide may be acylated. Exemplary T helper peptides include tetanus toxoid 830-843, influenza 307-319, malaria circumsporozoite 382-398 and 378-389

Multiple neoantigenic peptides described herein can also be linked together, optionally by a spacer.

### Peptide Productions, Polynucleotides and Vectors

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

In a further aspect the present disclosure provides a nucleic acid (e.g. polynucleotide) encoding a neoantigenic peptide as herein disclosed. The polynucleotide may be selected from DNA, cDNA, PNA, CNA, RNA, either single- and/or double-stranded, or native or stabilized forms of polynucleotides, such as for example polynucleotides with a phosphorothiate backbone, or combinations thereof and it may or may not contain introns so long as it codes for the peptide. Only peptides that contain naturally occurring amino acid residues joined by naturally occurring peptide bonds are encodable by a polynucleotide.

A still further aspect of the disclosure provides an expression vector capable of expressing a neoantigenic peptide as herein disclosed. Expression vectors for different cell types are well known in the art and can be selected without undue experimentation. Generally, the DNA is inserted into an expression vector, such as a plasmid, in proper orientation and correct reading frame for expression. The expression vector will comprise the appropriate heterologous transcriptional and/or translational regulatory control nucleotide sequences recognized by the desired host. The polynucleotide encoding the tumor neoantigenic peptide may be linked to such heterologous regulatory control nucleotide sequences or may be non-adjacent yet operably linked to such heterologous regulatory control nucleotide sequences. The vector is then introduced into the host through standard techniques. Guidance can be found for example in Sambrook et al. (1989) Molecular Cloning, A Laboratory Manual, Cold Spring Harbor Laboratory, Cold Spring Harbor, N.Y.

### Antigen Presenting Cells (APCs)

The present disclosure also encompasses a population of antigen presenting cells that have been pulsed with one or more of the peptides as previously defined and/or obtainable in a method as previously described. Preferably, the antigen presenting cells are dendritic cell (DCs) or artificial antigen presenting cells (aAPCs) (see Neal, Lillian R et al. “The Basics of Artificial Antigen Presenting Cells in T Cell-Based Cancer Immunotherapies.” Journal of immunology research and therapy vol. 2, 1 (2017): 68-79). Dendritic cells (DC) are professional antigen-presenting cells (APC) that have an extraordinary capacity to stimulate naive T-cells and initiate primary immune responses to pathogens. Indeed, the main role of mature DCs are to sense antigens and produce mediators that activate other immune cells, particularly T cells. DCs are potent stimulators for lymphocyte activation as they express MHC molecules that trigger TCRs (signal 1) and co-stimulatory molecules (signal 2) on T cells. Additionally, DCs also secrete cytokines that support T cell expansion. T cells require presented antigen in the form of a processed peptide to recognize foreign pathogens or tumor. Presentation of peptide epitopes derived from pathogen/tumor proteins is achieved through MHC molecules. MHC class I (MHC-I) and MHC class II (MHC-II) molecules present processed peptides to CD8+ T cells and CD4+ T cells, respectively. Importantly, DCs home to inflammatory sites containing abundant T cell populations to foster an immune response. Thus, DCs can be a crucial component of any immunotherapeutic approach, as they are intimately involved with the activation of the adaptive immune response. In the context of vaccines, DC therapy can enhance T cell immune responses to a desired target in healthy volunteers or patients with infectious disease or cancer. In one embodiment, APCS are artificial APC, which are genetically modified to express the desired T-cell co-stimulatory molecules, human HLA alleles and/or cytokines. Such artificial antigen presenting cells (aAPC) are able to provide the requirements for adequate T-cell engagement, co-stimulation, as well as sustained release of cytokines that allow for controlled T-cell expansion. These cells are not subject to the constraints of time and limited availability and can be stored in small aliquots for subsequent use in generating T-cell lines from different donors, thus representing an off the shelf reagent for immunotherapy applications. Expression of potent co-stimulatory signals on these aAPC endows this system with higher efficiency lending to increased efficacy of adoptive immunotherapy. Furthermore, aAPC can be engineered to express genes directing release of specific cytokines to facilitate the preferential expansion of desirable T-cell subsets for adoptive transfer; such as long lived memory T-cells (see for review Hasan A H et al., Artificial Antigen Presenting Cells: An Off the Shelf Approach for Generation of Desirable T-Cell Populations for Broad Application of Adoptive Immunotherapy; Adv Genet Eng. 2015; 4 (3): 130, Kim J V, Latouche J B, Rivière I, Sadelain M. The ABCs of artificial antigen presentation. Nat Biotechnol. 2004; 22:403-410 or Wang C, Sun W, Ye Y, Bomba H N, Gu Z. Bioengineering of Artificial Antigen Presenting Cells and Lymphoid Organs. Theranostics 2017; 7 (14): 3504-3516.).

Typically, the dendritic cells are autologous dendritic cells that are pulsed with a neoantigenic peptide as herein disclosed. The peptide may be any suitable peptide that gives rise to an appropriate T-cell response. The antigen-presenting cell (or stimulator cell) typically has an MHC class I or II molecule on its surface, and in one embodiment is substantially incapable of itself loading the MHC class I or II molecule with the selected antigen. The MHC class I or II molecule may readily be loaded with the selected antigen in vitro.

As an alternative the antigen presenting cell may comprise an expression construct encoding a tumor neoantigenic peptide as herein disclosed. The polynucleotide may be any suitable polynucleotide as previously defined and it is preferred that it is capable of transducing the dendritic cell, thus resulting in the presentation of a peptide and induction of immunity

Thus the present disclosure encompasses a population of APCs than can be pulsed or loaded with the neoantigenic peptide as herein disclosed, genetically modified (via DNA or RNA transfer) to express at least one neoantigenic peptide as herein disclosed, or that comprise an expression construct encoding a tumor neoantigenic peptide of the present disclosure. Typically the population of APCs is pulsed or loaded, modified to express or comprises at least one, at least 5, at least 10, at least 15, or at least 20 different neoantigenic peptide or expression construct encoding it.

The present disclosure also encompasses compositions comprising APCs as herein disclosed. APCs can be suspended in any known physiologically compatible pharmaceutical carrier, such as cell culture medium, physiological saline, phosphate-buffered saline, cell culture medium, or the like, to form a physiologically acceptable, aqueous pharmaceutical composition. Parenteral vehicles include sodium chloride solution, Ringer's dextrose, dextrose and sodium chloride, lactated Ringer's. Other substances may be added as desired such as antimicrobials. As used herein, a “carrier” refers to any substance suitable as a vehicle for delivering an APC to a suitable in vitro or in vivo site of action. As such, carriers can act as an excipient for formulation of a therapeutic or experimental reagent containing an APC. Preferred carriers are capable of maintaining an APC in a form that is capable of interacting with a T cell. Examples of such carriers include, but are not limited to water, phosphate buffered saline, saline, Ringer's solution, dextrose solution, serum-containing solutions, Hank's solution and other aqueous physiologically balanced solutions or cell culture medium. Aqueous carriers can also contain suitable auxiliary substances required to approximate the physiological conditions of the recipient, for example, enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer.

### Vaccine Compositions

The present disclosure further encompasses a vaccine or immunogenic composition capable of raising a specific T-cell response comprising:
one or more neoantigenic peptides as herein defined,
one or more polynucleotides encoding a neoantigenic peptide as herein defined; and/or
a population of antigen presenting cells (such as autologous dendritic cells or artificial APC) as described above.

Preferably, neoantigenic peptide which are encoded by tumor specific fusions as previously defined are used in vaccine compositions as per the present disclosure. Said neoantigenic peptide can be also named tumor specific peptides. Preferably also polynucleotides encoding tumor specific peptides are used as per the present disclosure.

A suitable vaccine or immunogenic composition will preferably contain between 1 and 20 neoantigenic peptides, more preferably 2, 3, 4, 5, 6, 7, 8, 9, 10, 11, 12, 13, 14, 15, 16, 17, 18, 19, 20, 21, 22, 23, 24, 25 different neoantigenic peptides, further preferred 6, 7, 8, 9, 10 11, 12, 13, or 14 different neoantigenic peptides, and most preferably 12, 13 or 14 different neoantigenic peptides.

The neoantigenic peptide(s) may be linked to a carrier protein. Where the composition contains two or more neoantigenic peptides, the two or more (e.g. 2-25) peptides may be linearly linked by a spacer molecule as described above, e.g. a spacer comprising 2-6 nonpolar or neutral amino acids.

In one embodiment of the present disclosure the different neoantigenic peptides, encoding polynucleotides, vectors, or APCs are selected so that one vaccine or immunogenic composition comprises neoantigenic peptides capable of associating with different MHC molecules, such as different MHC class I molecules. Preferably, such neoantigenic peptides are capable of associating with the most frequently occurring MHC class I molecules, e.g. different fragments capable of associating with at least 2 preferred, more preferably at least 3 preferred, even more preferably at least 4 preferred MHC class I molecules. In some embodiments, the compositions comprise peptides, encoding polynucleotides, vectors, or APCs capable of associating with one or more MHC class II molecules. The MHC is optionally HLA-A, -B, -C, -DP, -DQ, or -DR.

The vaccine or immunogenic composition is capable of raising a specific cytotoxic T-cells response and/or a specific helper T-cell response.

Thus, in a particular embodiment, the present disclosure also relates to a neoantigenic peptide as described above, wherein the neoantigenic peptide has a tumor specific neoepitope and is included in a vaccine or immunogenic composition. A vaccine composition is to be understood as meaning a composition for generating immunity for the prophylaxis and/or treatment of diseases. Accordingly, vaccines are medicines which comprise or generate antigens and are intended to be used in humans or animals for generating specific defense and protective substance by vaccination. An “immunogenic composition” is to be understood as meaning a composition that comprises or generates antigen(s) and is capable of eliciting an antigen-specific humoral or cellular immune response, e.g. T-cell response.

In a preferred embodiment, the neoantigenic peptide according to the disclosure is 8 or 9 residues long, or from 13 to 25 residues long. When the peptide is less than 20 residues, in order to have a peptide better suited for in vivo immunization, said neoantigenic peptide, is optionally flanked by additional amino acids to obtain an immunization peptide of more amino acids, usually more than 20.

Pharmaceutical compositions (i.e., the vaccine or immunogenic composition) comprising a peptide as herein described may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition by measuring specific CTL activity in the patient's blood. It must be kept in mind that the peptide and compositions of the present invention may generally be employed in serious disease states, that is, life-threatening or potentially life threatening situations, especially when the cancer has metastasized. In such cases, in view of the minimization of extraneous substances and the relative nontoxic nature of the peptide, it is possible and may be felt desirable by the treating physician to administer substantial excesses of these peptide compositions.

For therapeutic use, administration should begin at the detection or surgical removal of tumors. This is followed by boosting doses until at least symptoms are substantially abated and for a period thereafter.

The vaccine or immunogenic compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor.

The vaccine or immunogenic composition may be a pharmaceutical composition which additionally comprises a pharmaceutically acceptable adjuvant, immunostimulatory agent, stabilizer, carrier, diluent, excipient and/or any other materials well known to those skilled in the art. Such materials should be non-toxic and should not interfere with the efficacy of the active ingredient. The carrier is preferably an aqueous carrier but its precise nature of the carrier or other material will depend on the route of administration. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may further contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. See, for example, Butterfield, B M J. 2015 22; 350 for a discussion of cancer vaccines.

Example adjuvants that increase or expand the immune response of a host to an antigenic compound include emulsifiers, muramyl dipeptides, avridine, aqueous adjuvants such as aluminum hydroxide, chitosan-based adjuvants, saponins, oils, Amphigen, LPS, bacterial cell wall extracts, bacterial DNA, CpG sequences, synthetic oligonucleotides, cytokines and combinations thereof. Emulsifier include, for example, potassium, sodium and ammonium salts of lauric and oleic acid, calcium, magnesium and aluminum salts of fatty acids, organic sulfonates such as sodium lauryl sulfate, cetyltrhethylammonlum bromide, glycerylesters, polyoxyethylene glycol esters and ethers, and sorbitan fatty acid esters and their polyoxyethylene, acacia, gelatin, lecithin and/or cholesterol. Adjuvants that comprise an oil component include mineral oil, a vegetable oil, or an animal oil. Other adjuvants include Freund's Complete Adjuvant (FCA) or Freund's Incomplete Adjuvant (FIA). Cytokines useful as additional immunostimulatory agents include interferon alpha, interleukin-2 (IL-2), and granulocyte macrophage-colony stimulating factor (GM-CSF), or combinations thereof.

The concentration of peptides as herein described in the vaccine or immunogenic formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The peptides as herein described may also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), USA U.S. Pat. Nos. 4,235,871, 4,501,728 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and antigen presenting cell (APC) is present. Correspondingly, it may enhance the immune response if not only the peptide is used for activation of CTLs, but if additionally APCs with the respective MHC molecule are added. Therefore, in some embodiments the vaccine or immunogenic composition according to the present disclosure alternatively or additionally contains at least one antigen presenting cell, preferably a population of APCs.

The vaccine or immunogenic composition may thus be delivered in the form of a cell, such as an antigen presenting cell, for example as a dendritic cell vaccine. The antigen presenting cells such as a dendritic cell may be pulsed or loaded with a neoantigenic peptide as herein disclosed, may comprise an expression construct encoding a neoantigenic peptide as herein disclosed, or may be genetically modified (via DNA or RNA transfer) to express one, two or more of the herein disclosed neoantigenic peptides, for example at least 2, 3, 4, 5, 6, 7, 8, 9, or 10 neoantigenic peptides.

Suitable vaccines or immunogenic compositions may also be in the form of DNA or RNA relating to neoantigenic peptides as described herein. For example, DNA or RNA encoding one or more neoantigenic peptides or proteins derived therefrom may be used as the vaccine, for example by direct injection to a subject. For example, DNA or RNA encoding at least 2, 3, 4, 5, 6, 7, 8, 9, 10, 11, 12, 13, 14, 15, 16, 17, 18, 19, 20, 21, 22, 23, 24 or 25 neoantigenic peptides or proteins derived therefrom.

A number of methods are conveniently used to deliver the nucleic acids to the patient. For instance, the nucleic acid can be delivered directly, as “naked DNA”. This approach is described, for instance, in Wolff et al., Science 247:1465-1468 (1990) as well as USAU.S. U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6 (7): 682-691 (1988); 5279833USARose U.S. Pat. Nos. 5,279,833; 9,106,309WOAWO 91/06309; and Felgner et al., Proc. Natl. Acad. Sci. USA 84:7413-7414 (1987).

Delivery systems may optionally include cell-penetrating peptides, nanoparticulate encapsulation, virus like particles, liposomes, or any combination thereof. Cell penetrating peptides include TAT peptide, herpes simplex virus VP22, transportan, Antp. Liposomes may be used as a delivery system. Listeria vaccines or electroporation may also be used.

The one or more neoantigenic peptides may also be delivered via a bacterial or viral vector containing DNA or RNA sequences which encode one or more neoantigenic peptides. The DNA or RNA may be delivered as a vector itself or within attenuated bacteria virus or live attenuated virus, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides of the invention, e.g., Salmonella typhivectors and the like, will be apparent to those skilled in the art from the description herein.

An appropriate mean of administering nucleic acids encoding the peptides as herein described involves the use of minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Thus, the DNA or RNA encoding the neoantigenic peptide(s) may typically be operably linked to one or more of:
a promoter that can be used to drive nucleic acid molecule expression. AAV ITR can serve as a promoter and is advantageous for eliminating the need for an additional promoter element. For ubiquitous expression, the following promoters can be used: CMV (notably human cytomegalovirus immediate early promoter (hCMV-IE)), CAG, CBh, PGK, SV40, RSV, Ferritin heavy or light chains, etc. For brain expression, the following promoters can be used: Synapsin1 for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA). Typically, the promoter includes a down-stream cloning site for minigene insertion. For examples of suitable promoters sequences, see notably U.S. Pat. Nos. 5,580,859 and 5,589,466.
Transcriptional transactivators or other enhancer elements, which can also increase transcription activity, e.g. the regulatory R region from the 5′ long terminal repeat (LTR) of human T-cell leukemia virus type 1 (HTLV-1) (which when combined with a CMV promoter has been shown to induce higher cellular immune response).
Translation optimizing sequences e.g. a Kozak sequence flanking the AUG initiator codon (ACCAUGG) within mRNA, and codon optimization.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immunostimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used.

DNA vaccines or immunogenic compositions as herein described can be enhanced by co-delivering cytokines that promote cell-mediated immune responses, such as IL-2, IL-12, IL-18, GM-CSF and IFNγ. CXC chemokines such as IL-8, and CC chemokines such as macrophage inflammatory protein (MIP)-1α, MIP-3α, MIP-3β, and RANTES, may increase the potency of the immune response. DNA vaccine immunogenicity can also be enhanced by co-delivering plasmid-encoded cytokine-inducing molecules (e.g. LeIF), co-stimulatory and adhesion molecules, e.g. B7-1 (CD80) and/or B7-2 (CD86). Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Vaccines or immunogenic compositions comprising peptides may be administered in combination with vaccines or immunogenic compositions comprising polynucleotide encoding the peptides. For example, administration of peptide vaccine and DNA vaccine may be alternated in a prime-boost protocol. For example, priming with a peptide immunogenic composition and boosting with a DNA immunogenic composition is contemplated, as is priming with a DNA immunogenic composition and boosting with a peptide immunogenic composition.

The present disclosure also encompasses a method for producing a vaccine composition comprising the steps of:
a) Optionally, identifying at least one neoantigenic peptide according to the method as previously described;
b) producing said at least one neoantigenic peptide, at least one polypeptide encoding neoantigenic peptide(s), or at least a vector comprising said polypeptide(s) as described herein; and
c) optionally adding physiologically acceptable buffer, excipient and/or adjuvant and producing a vaccine with said at least one neoantigenic peptide, polypeptide or vector.

Another aspect of the present disclosure, is a method for producing a DC vaccine, wherein said DCs present at least one neoantigenic peptide as herein disclosed.

### Antibodies TCRs, CARs and Derivatives Thereof

The present disclosure also relates to an antibody or an antigen-binding fragment thereof that specifically binds a neoantigenic peptide as herein defined.

In some embodiments, the neoantigenic peptide is in association with an MHC or HLA molecule.

Typically, said antibody, or antigen-binding fragment thereof binds a neoantigenic peptide as herein defined, alone or optionally in association with an MHC or HLA molecule (i.e., peptide MHC complex) with a dissociation constant (K_{d}) of about 2×10⁻⁷M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10″ 9 M or less, about 3×10⁻⁹ or less, about 2×10⁻⁹ M or less, or about 1×10⁻⁹ M or less, or about 1×10⁻¹⁰ M or less, or about 1×10⁻¹² M or less. In certain non-limiting embodiments, the K_{d} is from about 1.5×10⁻⁶ M to about 2.7×10⁻¹² M.

The present disclosure thus includes antibodies targeting MHC-restricted peptides and in particular targeting neoantigenic peptides as herein defined in association with at an MHC (or HLA) molecule (peptide MHC complex) or TCR-like antibodies (see notably for detailed description and method of production Høydahl, Lene Støkken et al. “Targeting the MHC Ligandome by Use of TCR-Like Antibodies.” Antibodies (Basel, Switzerland) vol. 8, 2 32. 9 May. 2019, doi: 10.3390/antib8020032; He, Qinghua et al. “TCR-like antibodies in cancer immunotherapy.” Journal of hematology & oncology vol. 12, 1 99. 14 Sep. 2019, doi: 10.1186/s13045-019-0788-4; Trenevska I, Li D, Banham A H. Therapeutic Antibodies against Intracellular Tumor Antigens. Front Immunol. 2017 Aug. 18; 8:1001. doi: 10.3389/fimmu.2017.01001. PMID: 28868054; PMCID: PMC5563323; or. Chang A Y, Gejman R S, Brea E J, Oh C Y, Mathias M D, Pankov D, Casey E, Dao T, Scheinberg D A. Opportunities and challenges for TCR mimic antibodies in cancer therapy. Expert Opin Biol Ther. 2016 August; 16 (8): 979-87. doi: 10.1080/14712598.2016.1176138. Epub 2016 Apr. 27. PMID: 27094818; PMCID: PMC4936943).

In some embodiments, TCR-like antibodies can also be conjugated with cytotoxic organic compounds, such as antibody-drug conjugates (ADCs), radionuclides, and protein toxins, to mediate the specific killing of tumor cells (Dao T, et al. Therapeutic bispecific T-cell engager antibody targeting the intracellular oncoprotein WT1. Nat Biotechnol. 2015; 33 (10): 1079-1086). Furthermore, immunomodulators or secondary antibodies can be conjugated with the TCR-like antibodies to mediate specific immune responses around the tumor site, as in bi-specific T cell engagers (BiTE) (Trenevska I, Li D, Banham A H. Therapeutic antibodies against intracellular tumor antigens. Front Immunol. 2017; 8:1001).

To promote the infiltration and recognition of tumor cells by lymphocytes T (LT), another strategy consists in using antibodies capable of recognizing more than one antigenic target simultaneously and more particularly two antigenic targets simultaneously. There are many formats of bispecific antibodies. BiTE (bi-specific T-cell engager) are the first to have been developed. These are proteins of fusion consisting of two scFvs (variable domains heavy VH and light VL chains) from two antibodies linked by a binding peptide: one recognizes the LT marker (CD3+) and the other a tumor antigen. The goal is to favor recruitment and activation of LTs in contact with tumor, thus leading to cell lysis tumor (See for review Patrick A. Baeuerle and Carsten Reinhardt; Bispecific T-Cell Engaging Antibodies for Cancer Therapy; Cancer Res 2009; 69: (12). Jun. 15, 2009; and Galaine et al., Innovations & Thérapeutiques en Oncologie, vol. 3-n°3-7, mai-août 2017).

In a particular embodiment, said antibody is thus a bi-specific T-cell engager (BiTE), typically derived from a TCR-like antibody that specifically binds a tumor neoantigenic peptide as herein defined, optionally in association with a MHC or an HLA molecule and which further targets at least an immune cell antigen. Typically, the immune cell is a T cell, a NK cell or a dendritic cell. In this context, the targeted immune cell antigen may be for example CD3, CD16, CD30 or a TCR.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rlgG) fragments, variable heavy chain (VH) regions capable of specifically binding the antigen, single chain antibody fragments, including single chain variable fragments (scFv), and single domain antibodies (e.g., VHH antibodies, sdAb, sdFv, nanobody) fragments. The term encompasses genetically engineered and/or otherwise variants modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody and fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments, the antibody comprises a light chain variable domain and a heavy chain variable domain, e.g. in an scFv format.

Antibodies include variant polypeptide species that have one or more amino acid substitutions, insertions, or deletions in the native amino acid sequence, provided that the antibody retains or substantially retains its specific binding function. Conservative substitutions of amino acids are well known and described above.

The present disclosure further includes a method of producing an antibody, or antigen-binding fragment thereof, comprising a step of selecting antibodies that bind to a tumor neoantigen peptide as herein defined, optionally in association with an MHC or HLA molecule, with with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less, or about 1×10⁻¹⁰ M or less, or about 1×10⁻¹² M or less.

In certain embodiments, the antibody is of murine, human or camelid (e.g., lama) origin.

In some embodiments, the antibodies are selected from a library of human antibody sequences. In some embodiments, the antibodies are generated by immunizing an animal with a polypeptide comprising the neoantigenic peptide, optionally in association with an MHC or HLA molecule, followed by the selection step.

Antibodies including chimeric, humanized or human antibodies can be further affinity matured and selected as described above. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci.

Antibodies produced by said method, as well as immune cells expressing such antibodies or fragments thereof are also encompassed by the present disclosure.

The present disclosure also encompasses pharmaceutical compositions comprising one or more antibodies as herein disclosed alone or in combination with at least one other agent, such as a stabilizing compound, which may be administered in any sterile, biocompatible pharmaceutical carrier and optionally formulated with formulated with sterile pharmaceutically acceptable buffer(s), diluent(s), and/or excipient(s). Pharmaceutically acceptable carriers typically enhance or stabilize the composition, and/or can be used to facilitate preparation of the composition. Pharmaceutically acceptable carriers include solvents, dispersion media, coatings, antibacterial and antifungal agents, isotonic and absorption delaying agents, and the like that are physiologically compatible and in some embodiments pharmaceutically inert.

Administration of a pharmaceutical composition comprising antibodies as herein disclosed can be accomplished orally or parenterally. Methods of parenteral delivery include topical, intra-arterial (directly to the tumor), intramuscular, spinal, subcutaneous, intramedullary, intrathecal, intraventricular, intravenous, intraperitoneal, or intranasal administration.

Thus, in addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Depending on the route of administration, the active compound, i.e., antibody, bispecific and multispecific molecule, may be coated in a material to protect the compound from the action of acids and other natural conditions that may inactivate the compound.

The composition is typically sterile and preferably fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxilliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl, cellulose, hydroxypropylmethylcellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, ie. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

Pharmaceutical compositions of the disclosure can be prepared in accordance with methods well known and routinely practiced in the art. See. e.g., Remington: The Science and Practice of Pharmacy, Mack Publishing Co., 20th ed., 2000; and Sustained and Controlled Release Drug Delivery Systems, J R. Robinson, ed., Marcel Dekker, Inc., New York, 1978. Pharmaceutical compositions are preferably manufactured under GMP conditions.

The present disclosure also encompasses a recombinant T cell receptor (TCR) that targets a neoantigenic peptide as herein defined in association with an MHC or HLA molecule.

The present disclosure further includes a method of producing a TCR, or an antigen-binding fragment thereof, comprising a step of selecting TCRs that bind to a tumor neoantigen peptide as herein defined, optionally in association with an MHC or HLA molecule, with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less, or about 1×10⁻¹⁰ M or less, or about 1×10⁻¹² M or less.

Nucleic acid encoding the TCR can be obtained from a variety of sources, such as by polymerase chain reaction (PCR) amplification of naturally occurring TCR DNA sequences, followed by expression of antibody variable regions, followed by the selecting step described above. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

A “T cell receptor” or “TCR” refers to a molecule that contains a variable α and β chains (also known as TCRa and TCRb, respectively) or a variable γ and δ chains (also known as TCRg and TCRd, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules through its extracellular binding domain. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains {e.g., α-chain, β-chain) can contain two immunoglobulin domains, a variable domain {e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain {e.g., α-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD35 chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each CD35 chain has three. Generally, ITAMs are involved in the signaling capacity of the TCR complex. These accessory molecules have negatively charged transmembrane regions and play a role in propagating the signal from the TCR into the cell. The CD3-and-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

While T-cell receptors (TCRs) are transmembrane proteins and do not naturally exist in soluble form, antibodies can be secreted as well as membrane bound. Importantly, TCRs have the advantage over antibodies that they in principle can recognize peptides generated from all degraded cellular proteins, both intra- and extracellular, when presented in the context of MHC molecules. Thus TCRs have important therapeutic potential.

The present disclosure also relates to soluble T-cell receptors (sTCRs) that contain the antigen recognition part directed against a tumor neoantigenic peptide as herein disclosed (see notably Walseng E, Wälchli S, Fallang L-E, Yang W, Vefferstad A, Areffard A, et al. (2015) Soluble T-Cell Receptors Produced in Human Cells for Targeted Delivery. PLOS ONE 10 (4): e0119559). In a particular embodiment, the soluble TCR can be fused to an antibody fragment directed to a T cell antigen, optionally wherein the targeted antigen is CD3 or CD16 (see for example Boudousquie, Caroline et al. “Polyfunctional response by ImmTAC (IMCgp100) redirected CD8+ and CD4+ T cells.” Immunology vol. 152, 3 (2017): 425-438. doi: 10.1111/imm.12779).

In certain embodiments, the present disclosure encompasses Recombinant HLA-independent (or non-HLA restricted) T cell receptors (referred to as “HI-TCRs”) that bind to a neoantigenic peptide as herein defined in an HLA-independent manner. “HI-TCRs” as herein intended and which are well-suited to the present invention are described in International Application No. WO 2019/157454. Thus, typically HI-TCRs according to the present disclosure comprise an antigen binding chain that comprises: (a) an antigen-binding domain (as previously defined) that binds to an antigen in an HLA-independent manner, for example, an antigen-binding fragment of an immunoglobulin variable region; and (b) a constant domain that is capable of associating with (and consequently activating) a CD32 polypeptide. Because typically TCRs bind antigen in a HLA-dependent manner, the antigen-binding domain that binds in an HLA-independent manner is heterologous. Preferably, the antigen-binding domain or fragment thereof comprises: (i) an antigen-binding domain comprising or consisting of an heavy chain variable region (VH) of an antibody and/or (ii) a light chain variable region (VL) of an antibody. The constant domain of the TCR is, for example, a native or modified TRAC polypeptide, or a native or modified TRBC polypeptide. The constant domain of the TCR is, for example, a native TCR constant domain (alpha or beta) or fragment thereof. Unlike chimeric antigen receptors, which typically themselves comprise an intracellular signaling domain, the HI-TCR does not directly produce an activating signal; instead, the antigen-binding chain associates with and consequently activates a CD3 polypeptide. The immune cells comprising the recombinant TCR provide superior activity when the antigen has a low density on the cell surface of less than about 10,000 molecules per cell.

The CD3ζ polypeptide is, for example, a native CD3ζ polypeptide or a modified CD3ζ polypeptide. The CD3ζ polypeptide is optionally fused to an intracellular domain of a co-stimulatory molecule or a fragment thereof. Alternatively, the antigen binding domain optionally comprises a co-stimulatory region, e.g. intracellular domain, that is capable of stimulating an immunoresponsive cell upon the binding of the antigen binding chain to the antigen. Example co-stimulatory molecules include CD28, 4-1BB, OX40, ICOS, DAP-10, fragments thereof, or a combination thereof.

In some embodiments, the recombinant HI-TCR is expressed by a transgene that is integrated at an endogenous gene locus of the immunoresponsive cell, for example, a CD3δ locus, a CD3ε locus, a CD247 locus, a B2M locus, a TRAC locus, a TRBC locus, a TRDC locus and/or a TRGC locus. In most embodiments, expression of the recombinant HI-TCR is driven from the endogenous TRAC or TRBC gene locus. In some embodiments, the transgene encoding a portion of the recombinant HI-TCR is integrated into the endogenous TRAC and/or TRBC locus in a manner that disrupts or abolishes the endogenous expression of a TCR comprising a native TCR α chain and/or a native TCR β chain. This disruption prevents or eliminates mispairing between the recombinant TCR and a native TCR α chain and/or a native TCR β chain in the immunoresponsive cell. The endogenous gene locus may also comprise a modified transcription terminator region, for example, a TK transcription terminator, a GCSF transcription terminator, a TCRA transcription terminator, an HBB transcription terminator, a bovine growth hormone transcription terminator, an SV40 transcription terminator, and a P2A element.

In some embodiments of the present disclosure, the recombinant TCR and typically the HI-TCR comprises an extracellular antigen-binding domain which is capable of dimerizing with a second extracellular antigen-binding domain. Typically, the second extracellular antigen-binding domain binds a tumor antigen, preferably wherein the tumor antigen is selected from pHER95, CD19, MUC16, MUC1, CAIX, CEA, CD8, CD7, CD10, CD20, CD22, CD30, CD70, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-a, GD2, GD3, HER-2, hTERT, IL-13R-a2, κ-light chain, KDR, LeY, L1 cell adhesion molecule, MAGE-A1, Mesothelin, MAGEA3, p53, MART1, GP100, Proteinase3 (PRI), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, LILRB4, PRAME, and ERBB.

The present disclosure also encompasses a chimeric antigen receptor (CAR) which is directed against a tumor neoantigenic peptide as herein disclosed. CARs are fusion proteins comprising an antigen-binding domain, typically derived from an antibody, linked to the signalling domain of the TCR complex. CARs can be used to direct immune cells, such as T-cells or NK T cells, against a tumor neoantigenic peptide as previously defined with a suitable antigen-binding domain selected.

The antigen-binding domain of a CAR is typically based on a scFv (single chain variable fragment) derived from an antibody. In addition to an N-terminal, extracellular antibody-binding domain, CARs typically may comprise a hinge domain, which functions as a spacer to extend the antigen-binding domain away from the plasma membrane of the immune effector cell on which it is expressed, a transmembrane (TM) domain, an intracellular signalling domain (e.g. the signalling domain from the zeta chain of the CD3 molecule (CD35) of the TCR complex, or an equivalent) and optionally one or more co-stimulatory domains which may assist in signalling or functionality of the cell expressing the CAR. Signalling domains from co-stimulatory molecules including CD28, OX-40 (CD134), and 4-1BB (CD137) can be added alone (second generation) or in combination (third generation) to enhance survival and increase proliferation of CAR modified T cells. Potential co-stimulatory domains also include ICOS-1, CD27, GITR, and DAP10.

Thus, the CAR may include
(1) In its extracellular portion, one or more antigen binding molecules, such as one or more antigen-binding fragment, domain, or portion of an antibody, or one or more antibody variable domains, and/or antibody molecules.
(2) In its transmembrane portion, a transmembrane domain derived from human T cell receptor-alpha or -beta chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR. In some embodiments, the transmembrane domain is derived from CD28, CD8 or CD3-zeta.
(3) One or more co-stimulatory domains, such as co-stimulatory domains derived from human CD28, 4-1BB (CD137), ICOS-1, CD27, OX 40 (CD137), DAP10, and GITR (AITR). In some embodiments, the CAR comprises co-stimulating domains of both CD28 and 4-1BB.
(4) In its intracellular signalling domain, an intracellular signalling domain comprising one or more ITAMs, for example, the intracellular signalling domain is CD3-zeta, or a variant thereof lacking one or two ITAMs (e.g. ITAM3 and ITAM2), or the intracellular signalling domain is derived from FcεRIγ.

The CAR can be designed to recognize tumor neoantigenic peptide alone or in association with an HLA or MHC molecule.

The moieties used to bind to antigen include three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3 (4): 388-398 (see notably table 1) and are included in the present application.

Antibodies include chimeric, humanized or human antibodies, and can be further affinity matured and selected as described above. Chimeric or humanized scFv's derived from rodent immunoglobulins (e.g. mice, rat) are commonly used, as they are easily derived from well-characterized monoclonal antibodies. Humanized antibodies contain rodent-sequence derived CDR regions; typically, the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequences, and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

Typically, the CAR includes an antigen-binding domain as previously defined from an antibody molecule, such as a single-chain antibody fragment (scFv) derived from the variable heavy (VH) and variable light (VL) chains of a monoclonal antibody (mAb).

In some aspects, the antigen-binding, domain of the CAR is linked to one or more transmembrane and intracellular signaling domains. In some embodiments, the CAR includes a transmembrane domain fused to the extracellular domain of the CAR. In one embodiment, the transmembrane domain that is naturally associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain can be derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR). The transmembrane domain can also be synthetic. In some embodiments, the transmembrane domain is derived from CD28, CD8 or CD3-zeta.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3-chain, which is the primary transmitter of signals from endogenous TCRs. Second generation CARs typically further comprise intracellular signaling domains from various costimulatory protein receptors (e.g., CD28, 41BB (CD28), ICOS) to the cytoplasmic tail of the CAR to provide additional signals to the T cell. Co-stimulatory domains include domains derived from human CD28, 4-1BB (CD137), ICOS-1, CD27, OX 40 (CD137), DAP10, and GITR (AITR). Combinations of two co-stimulatory domains are contemplated, e.g. CD28 and 4-1BB, or CD28 and OX40. Third generation CARs combine multiple signaling domains, such as CD3z-CD28-4-1BB or CD3z-CD28-OX40, to augment potency.

The intracellular signaling domain can be from an intracellular component of the TCR complex, such as a TCR CD3+chain that mediates T-cell activation and cytotoxicity, e.g., the CD3 zeta chain. Alternative intracellular signaling domains include FcεRIγ. The intracellular signaling domain may comprise a modified CD3 zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The intracellular signaling region of CD3-zeta is residues 22-164 of SEQ ID NO: 4. ITAM1 is located around amino acid residues 61-89, ITAM2 around amino acid residues 100-128, and ITAM3 around residues 131-159. Thus, the modified CD3 zeta polypeptide may have any one of ITAM1, ITAM2, or ITAM3 inactivated. Alternatively, the modified CD3 zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3 zeta polypeptide retains only ITAM1 and the remaining CD32 domain is deleted (residues 90-164). As another example, ITAM1 is substituted with the amino acid sequence of ITAM3, and the remaining CD32 domain is deleted (residues 90-164). See, for example, Bridgeman et al., Clin. Exp. Immunol. 175 (2): 258-67 (2014); Zhao et al., J. Immunol. 183 (9): 5563-74 (2009); Maus et al., WO 2018/132506; Sadelain et al., WO/2019/133969, Feucht et al., Nat Med. 25 (1): 82-88 (2019).

Thus, in some aspects, the antigen binding domain is linked to one or more cell signaling modules. In some embodiments, cell signaling modules include CD3 transmembrane domain, CD3 intracellular signaling domains, and/or other CD transmembrane domains. The CAR can also further include a portion of one or more additional molecules such as Fc receptor γ, CD8, CD4, CD25, or CD16.

In some embodiments, upon ligation of the CAR, the cytoplasmic domain or intracellular signaling domain of the CAR activates at least one of the normal effector functions or responses of the corresponding non-engineered immune cell (typically a T cell). For example, the CAR can induce a function of a T cell such as cytolytic activity or T-helper activity, secretion of cytokines or other factors.

In some embodiments, the intracellular signaling domain(s) include the cytoplasmic sequences of the T cell receptor (TCR), and in some aspects also those of co-receptors that in the natural context act in concert with such receptor to initiate signal transduction following antigen-specific receptor engagement, and/or a variant of such molecules, and/or any synthetic sequence that has the same functional capability.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta

The CAR can also include a signaling domain and/or transmembrane portion of a costimulatory receptor, such as CD28, 4-1BB, OX40, DAP10, and ICOS. In some aspects, the same CAR includes both the activating and costimulatory components; alternatively, the activating domain is provided by one CAR whereas the costimulatory component is provided by another CAR recognizing another antigen.

Thus, in some embodiments, the CAR may include:
(1) In its extracellular portion, one or more antigen binding molecules, such as one or more antigen-binding fragment, domain, or portion of an antibody, or one or more antibody variable domains (heavy chain and/or light chain), and/or antibody molecules.
(2) In its transmembrane portion, a transmembrane domain derived from human T cell receptor-alpha or -beta chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR. In some embodiments, the transmembrane domain is derived from CD28, CD8 or CD3-zeta.
(3) One or more co-stimulatory domains, such as co-stimulatory domains derived from human CD28, 4-1BB (CD137), ICOS-1, CD27, OX 40 (CD137), DAP10, and GITR (AITR). In some embodiments, the CAR comprises co-stimulating domains of both CD28 and 4-1BB.
(4) In its intracellular signalling domain, one or more intracellular signalling domain(s) comprising one or more ITAMs, for example: the intracellular signalling domain or a portion thereof from CD3-zeta, or a variant thereof lacking one or two ITAMs (e.g.: ITAM3 and/or ITAM2 see also as detailed above and bibliographic references), FcR gamma, FcR beta, CD3 gamma, CD3 delta, CD3 epsilon, CDS, CD22, CD79a, CD79b, and/or CD66d, notably selected from the intracellular domain of CD3-zeta, or a variant thereof lacking one or two ITAMs (e.g.: ITAM3 and ITAM2), or the intracellular signalling of FcεRIγ or a variant thereof.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

In some embodiments, the CAR is a MHC-restricted antibody-based chimeric antigen receptor or TCR-like CAR. Typically, such CAR comprises an antibody or a fragment thereof targeting a MHC restricted neoantigenic peptide as previously defined. A non-limiting example of a CAR MHC-restricted antibody-based CAR which general structure is typically well-suited according to the present disclosure can be found in Maus MV, Plotkin J, Jakka G, et al. An MHC-restricted antibody-based chimeric antigen receptor requires TCR-like affinity to maintain antigen specificity. Mol Ther Oncolytics. 2017; 3:1-9.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061, WO2019157454, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3 (4): 388-398; Davila et al. (2013) PLOS ONE 8 (4): e61338; Turtle et al., Curr. Opin. Immunol., 2012 October; 24 (5): 633-39; Wu et al., Cancer, 2012 Mar. 18 (2): 160-75. In some aspects, the genetically engineered antigen receptors include a CAR as described in U.S. Pat. No. 7,446,190, and those described in International Patent Application Publication No.: WO/2014055668 A1.

The present disclosure also encompasses polynucleotides encoding antibodies, antigen-binding fragments or derivatives thereof, TCRs and CARs as previously described as well as vector comprising said polynucleotide(s).

### Immune Cells

The present disclosure further encompasses an immune cell, notably an isolated immune cell which target one or more tumor neoantigenic peptides as previously described. In more specific embodiments the present disclosure encompasses an immune cell, notably an isolated immune cell expressing a recombinant CAR or TCR as previously defined.

As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, the term “T cell” includes cells bearing a T cell receptor (TCR), in particular TCR directed against a tumor neoantigenic peptide as herein disclosed. T-cells according to the present disclosure can be selected from the group consisting of inflammatory T-lymphocytes, cytotoxic T-lymphocytes, regulatory T-lymphocytes, Mucosal-Associated Invariant T cells (MAIT), Yδ T cell, tumour infiltrating lymphocyte (TILs) or helper T-lymphocytes included both type 1 and 2 helper T cells and Th 17 helper cells. In another embodiment, said cell can be derived from the group consisting of CD4+ T-lymphocytes and CD8+ T-lymphocytes. Said immune cells may originate from a healthy donor or from a subject suffering from a cancer. In some embodiments, the immune cell is an allogenic or autologous cell. In some embodiments, the immune cell is selected from T cells, Natural Killer T cells, CD4+/CD8+ T cells, TILs/tumor derived CD8 T cells, central memory CD8+ T cells, Treg, MAIT, Yδ T cells, human embryonic stem cells, and pluripotent stem cells from which lymphoid cells may be differentiated.

Immune cells can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person, such as FICOLL™ separation. In one embodiment, cells from the circulating blood of a subject are obtained by apheresis. In certain embodiments, T-cells are isolated from PBMCs. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes, for instance by using CD14 DYNABEADS®. In some embodiments, red blood cells may be lysed prior to the density gradient centrifugation. In another embodiment, said cell can be derived from a healthy donor, from a subject diagnosed with cancer. The cell can be autologous or allogeneic.

In allogeneic immune cell therapy, immune cells are collected from healthy donors, rather than the patient. Typically these are HLA matched to reduce the likelihood of graft vs. host disease. Alternatively, universal ‘off the shelf’ products that may not require HLA matching comprise modifications designed to reduce graft vs. host disease, such as disruption or removal of the TCRαβ receptor. See Graham et al., Cells. 2018 October; 7 (10): 155 for a review. Because a single gene encodes the alpha chain (TRAC) rather than the two genes encoding the beta chain, the TRAC locus is a typical target for removing or disrupting TCRαβ receptor expression. Alternatively, inhibitors of TCRαβ signalling may be expressed, e.g. truncated forms of CD3% can act as a TCR inhibitory molecule. Disruption or removal of HLA class I molecules has also been employed. For example, Torikai et al., Blood. 2013; 122:1341-1349 used ZFNs to knock out the HLA-Alocus, while Ren et al., Clin. Cancer Res. 2017; 23:2255-2266 knocked out Beta-2 microglobulin (B2M), which is required for HLA class I expression. Ren et al. simultaneously knocked out TCRαβ, B2M and the immune-checkpoint PD1. Generally, the immune cells are activated and expanded to be utilized in the adoptive cell therapy. The immune cells as herein disclosed can be expanded in vivo or ex vivo. The immune cells, in particular T-cells can be activated and expanded generally using methods known in the art. Generally the T-cells are expanded by contact with a surface having attached thereto an agent that stimulates a CD3/TCR complex associated signal and a ligand that stimulates a co-stimulatory molecule on the surface of the T cells.

In one embodiment of the present disclosure, the immune cell can be modified to be directed to tumor neoantigenic peptides as previously defined. In a particular embodiment, said immune cell may express a recombinant antigen receptor directed to said neoantigenic peptide its cell surface. By “recombinant” is meant an antigen receptor which is not encoded by the cell in its native state, i.e. it is heterologous, non-endogenous. Expression of the recombinant antigen receptor can thus be seen to introduce new antigen specificity to the immune cell, causing the cell to recognise and bind a previously described peptide. The antigen receptor may be isolated from any useful source. In some embodiments, the cells comprise one or more nucleic acids introduced via genetic engineering that encode one or more antigen receptors, wherein the antigen include at least one tumor neoantigenic peptide as per the present disclosure.

Among the antigen receptors as per the present disclosure are genetically engineered T cell receptors (TCRs) and components thereof, as well as functional non-TCR antigen receptors, such as chimeric antigen receptors (CAR) as previously described.

Methods by which immune cells can be genetically modified to express a recombinant antigen receptor are well known in the art. A nucleic acid molecule encoding the antigen receptor may be introduced into the cell in the form of e.g. a vector, or any other suitable nucleic acid construct. Vectors, and their required components, are well known in the art. Nucleic acid molecules encoding antigen receptors can be generated using any method known in the art, e.g. molecular cloning using PCR. Antigen receptor sequences can be modified using commonly-used methods, such as site-directed mutagenesis.

In some embodiments, the immune cell is cell wherein the gene encoding the Suv39h1 protein, and in particular the human suv39h1 protein (referenced 043463 in UNIPROT), is disrupted.

In some embodiments, the immune cell comprises a recombinant HLA-independent (or non-HLA restricted) T cell receptors (referred to as “HI-TCRs”) that bind to an antigen of interest in an HLA-independent manner are described in International Application No. WO 2019/157454. Such HI-TCRs comprise an antigen binding chain that comprises: (a) an antigen-binding domain that binds to an antigen in an HLA-independent manner, for example, an antigen-binding fragment of an immunoglobulin variable region; and (b) a constant domain that is capable of associating with (and consequently activating) a CD32 polypeptide. Because typically TCRs bind antigen in a HLA-dependent manner, the antigen-binding domain that binds in an HLA-independent manner must be heterologous. Preferably, the antigen-binding domain or fragment thereof comprises: (i) a heavy chain variable region (VH) of an antibody and/or (ii) a light chain variable region (VL) of an antibody. The constant domain of the TCR is, for example, a native or modified TRAC polypeptide, or a native or modified TRBC polypeptide. The constant domain of the TCR is, for example, a native TCR constant domain (alpha or beta) or fragment thereof. Unlike chimeric antigen receptors, which typically themselves comprise an intracellular signaling domain, the HI-TCR does not directly produce an activating signal; instead, the antigen-binding chain associates with and consequently activates a CD3ζ polypeptide. The immune cells comprising the recombinant TCR provide superior activity when the antigen has a low density on the cell surface of less than about 10,000 molecules per cell.

The CD3ζ polypeptide is, for example, a native CD3ζ polypeptide or a modified CD3ζ polypeptide. The CD3ζ polypeptide is optionally fused to an intracellular domain of a co-stimulatory molecule or a fragment thereof. Alternatively, the antigen binding domain optionally comprises a co-stimulatory region, e.g. intracellular domain, that is capable of stimulating an immunoresponsive cell upon the binding of the antigen binding chain to the antigen. Example co-stimulatory molecules include CD28, 4-1BB, OX40, ICOS, DAP-10, fragments thereof, or a combination thereof.

In some embodiments of the present disclosure, the immune cell is a cell wherein (a) the SUV39H1 gene is inactivated, (b) the antigen-specific receptor is a modified TCR comprising a heterologous (or recombinant) antigen-binding domain and a native TCR constant domain or fragment thereof, and the antigen-specific receptor is capable of activating a CD3 zeta polypeptide. For example, the immune cell may further comprise at least one chimeric costimulatory receptor (CCR) and/or at least one chimeric antigen receptor, for example as previously defined.

In a related aspect, the immune cells, particularly if allogeneic, may be designed to reduce graft vs. host disease, such that the cells comprise inactivated (e.g. disrupted or deleted) TCRαβ receptor. In such cases, the nucleic acid encoding the antigen-binding domain of the HI-TCR (typically as previously defined) is conveniently inserted into the endogenous TRAC locus and/or TRBC locus of the immune cell. The insertion of the HI-TCR nucleic acid sequence, or another smaller mutation, can disrupt or abolish the endogenous expression of a TCR comprising a native TCR alpha chain and/or a native TCR beta chain. The insertion or mutation may reduce endogenous TCR expression by at least about 50%, 60%, 70%, 75%, 80%, 85%, 90%, 95%, 98%, or 99%. Because a single gene encodes the alpha chain (TRAC) rather than the two genes encoding the beta chain, the TRAC locus is a typical target for reducing TCRαβ receptor expression. Thus, the nucleic acid encoding the antigen-specific receptor (e.g. CAR or TCR) may be integrated into the TRAC locus at a location, preferably in the 5′ region of the first exon (SEQ ID NO: 3), that significantly reduces expression of a functional TCR alpha chain. See, e.g., Jantz et al., WO 2017/062451; Sadelain et al., WO 2017/180989; Torikai et al,. Blood, 119 (2): 5697-705 (2012); Eyquem et al., Nature. 2017 Mar. 2; 543 (7643): 113-117. Expression of the endogenous TCR alpha may be reduced by at least about 50%, 60%, 70%, 75%, 80%, 85%, 90%, 95%, 98%, or 99%. In such embodiments, expression of the nucleic acid encoding the antigen-specific receptor is optionally under control of the endogenous TCR-alpha or endogenous TCR-beta promoter.

Optionally, the immune cell also comprises a modified CD3 with a single active ITAM domain, and optionally the CD3 may further comprise one or more or two or more costimulatory domains. In some embodiments, the CD3 comprises two costimulatory domains, optionally CD28 and 4-1BB. The modified CD3 with a single active ITAM domain can comprise, for example, a modified CD3zeta intracellular signaling domain in which ITAM2 and ITAM3 have been inactivated, or ITAM1 and ITAM2 have been inactivated. In some embodiments, a modified CD3 zeta polypeptide retains only ITAM1 and the remaining CD32 domain is deleted (residues 90-164). As another example, ITAM1 is substituted with the amino acid sequence of ITAM3, and the remaining CD32 domain is deleted (residues 90-164).

The modified immune cells disclosed herein may comprise combinations of two or more, or three or more, or four or more, of the foregoing aspects.

For example, the modified immune cell is an immune cell wherein (a) the antigen-specific receptor is a modified TCR comprising a heterologous (or recombinant) antigen-binding domain (typically as previously defined) and a native TCR constant domain or fragment thereof, and the antigen-specific receptor is capable of activating a CD3 zeta polypeptide, and/or the antigen-specific receptor is a CAR, and optionally (b) the SUV39H1 gene is inactivated, and optionally (c) the immune cell comprises a modified CD3 with a single active ITAM domain, e.g. in which ITAM2 and ITAM3 have been inactivated, and optionally (d) the TCR is under control of an endogenous TRAC and/or TRBC promoter, and optionally (e) expression of native TCR-alpha chain and/or native TCR-beta chain are disrupted or abolished. In further embodiments, the cell may comprise at least one chimeric costimulatory receptor (CCR). In some embodiments, the tumor antigen is selected from pHER95, CD19, MUC16, MUC1, CAIX, CEA, CD8, CD7, CD10, CD20, CD22, CD30, CD70, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-a, GD2, GD3, HER-2, hTERT, IL-13R-a2, κ-light chain, KDR, LeY, L1 cell adhesion molecule, MAGE-A1, Mesothelin, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, LILRB4, PRAME, and ERBB.]

In some embodiments, the recombinant HI-TCR is expressed by a transgene that is integrated at an endogenous gene locus of the immunoresponsive cell, for example, a CD38 locus, a CD38 locus, a CD247 locus, a B2M locus, a TRAC locus, a TRBC locus, a TRDC locus and/or a TRGC locus. In most embodiments, expression of the recombinant HI-TCR is driven from the endogenous TRAC or TRBC gene locus. In some embodiments, the transgene encoding a portion of the recombinant HI-TCR is integrated into the endogenous TRAC and/or TRBC locus in a manner that disrupts or abolishes the endogenous expression of a TCR comprising a native TCR α chain and/or a native TCR β chain. This disruption prevents or eliminates mispairing between the recombinant TCR and a native TCR α chain and/or a native TCR β chain in the immunoresponsive cell. The endogenous gene locus may also comprise a modified transcription terminator region, for example, a TK transcription terminator, a GCSF transcription terminator, a TCRA transcription terminator, an HBB transcription terminator, a bovine growth hormone transcription terminator, an SV40 transcription terminator, and a P2A element.

The present disclosure also relates to a method for providing an immune cell, and in particular a T cell population which targets a tumor neoantigenic peptide as herein disclosed, in particular an immune cell and notably a T cell population expressing a TCR or a CAR in particular an MHC-restricted antibody-based chimeric antigen receptor as previously defined.

The T cell population may comprise CD8+ T cells, CD4+ T cells or CD8+ and CD4+ T cells.

Immune cell populations produced in accordance with the present disclosure may be enriched with immune cells that are specific to, i.e. target, the tumor neoantigenic peptide of the present disclosure. That is, the immune cell population that is produced in accordance with the present disclosure will have an increased number of immune cells that target one or more tumor neoantigenic peptide. For example, the immune cell population of the disclosure will have an increased number of immune cells that target a tumor neoantigenic peptide compared with the immune cells in the sample isolated from the subject. That is to say, the composition of the immune cell population will differ from that of a “native” immune cell population (i.e. a population that has not undergone the identification and expansion steps discussed herein), in that the percentage or proportion of immune cells that target a tumor neoantigenic peptide will be increased.

Immune cell populations produced in accordance with the present disclosure may be enriched with immune cells that are specific to, i.e. target, tumor neoantigenic peptide. That is, the T cell population that is produced in accordance with the present disclosure will have an increased number of immune cells that target one or more tumor neoantigenic peptide of the present disclosure. For example, the immune cell population of the present disclosure will have an increased number of immune cells that target a tumor neoantigenic peptide compared with the immune cells in the sample isolated from the subject. That is to say, the composition of the immune cell population will differ from that of a “native” immune cell population (i.e. a population that has not undergone the identification and expansion steps discussed herein), in that the percentage or proportion of immune cells that target a tumor neoantigenic peptide will be increased.

The immune cell population according to the present disclosure may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a tumor neoantigenic peptide as herein disclosed. For example, the immune cell population may have about 0.2%-5%, 5%-10%, 10-20%, 20-30%, 30-40%, 40-50%, 50-70% or 70-100% immune cells that target a tumor neoantigenic peptide of the present disclosure.

An expanded population of tumor neoantigenic peptide-reactive immune cells may have a higher activity than a population of immune cells not expanded, for example, using a tumor neoantigenic peptide. Reference to “activity” may represent the response of the immune cell population to restimulation with a tumor neoantigenic peptide, e.g. a peptide corresponding to the peptide used for expansion, or a mix of tumor neoantigenic peptide. Suitable methods for assaying the response are known in the art. For example, cytokine production may be measured (e.g. IL2 or IFNy production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

In a preferred embodiment present disclosure provides a plurality or population, i.e. more than one, of immune cells wherein the plurality of immune cells comprises a immune cell, notably a T cell, which recognizes a clonal tumor neoantigenic peptide and a T cell which recognizes a different clonal tumor neoantigenic peptide. As such, the present disclosure provides a plurality of immune cells, notably T cells, which recognize different clonal tumor neoantigenic peptide. Different immune cells, notably T cells, in the plurality or population may alternatively have different TCRs which recognize the same tumor neoantigenic peptide.

In a preferred embodiment the number of clonal tumor neoantigenic peptide recognized by the plurality of T cells is from 2 to 1000. For example, the number of clonal neo-antigens recognized may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, preferably 2 to 100. There may be a plurality of immune cells, notably T cells, with different TCRs but which recognize the same clonal neo-antigen.

The immune cell and in particular the T cell population may be all or primarily composed of CD8+ T cells, or all or primarily composed of a mixture of CD8+ T cells and CD4+ T cells or all or primarily composed of CD4+ T cells.

In particular embodiments, the T cell population is generated from T cells isolated from a subject with a tumor. For example, the T cell population may be generated from T cells in a sample isolated from a subject with a tumor. The sample may be a tumor sample, a peripheral blood sample or a sample from other tissues of the subject.

In a particular embodiment the immune cell population is generated from a sample from the tumor in which the tumor neoantigenic peptide is identified. In other words, the immune cell and notably the T cell population is isolated from a sample derived from the tumor of a patient to be treated. Such T cells are referred to herein as ‘tumor infiltrating lymphocytes’ (TILs).

T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient.

### Cancer Therapeutic Methods

In any of the embodiments, the Cancer Therapeutic Products described herein may be used in methods for inhibiting proliferation of cancer cells. The Cancer Therapeutic Products described herein may also be used in the treatment of cancer, in patients suffering from cancer, or for the prophylactic treatment of cancer, in patients at risk of cancer.

Cancers that can be treated using the therapy described herein include any solid or non-solid tumors as previously defined. Of particular interest according to the present disclosure are breast cancer, melanoma and lung cancer.

Cancers includes also the cancers which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

A subject as per the present disclosure is typically a patient in need thereof that has been diagnosed with cancer or is at risk of developing cancer. The subject is typically a human, dog, cat, horse or any animal in which a tumor specific immune response is desired.

The present disclosure also pertains to a neoantigenic peptide, a population of APCs, a vaccine or immunogenic composition, a polynucleotide encoding a neoantigenic peptide or a vector as previously defined for use in cancer vaccination therapy of a subject or for treating cancer in a subject, wherein the peptide(s) binds at least one MHC molecule of said subject.

The present disclosure also provides a method for treating cancer in a subject comprising administering a vaccine or immunogenic composition as described herein to said subject in a therapeutically effective amount to treat the subject. The method may additionally comprise the step of identifying a subject who has cancer.

The present disclosure also relates to a method of treating cancer comprising producing an antibody or antigen-binding fragment thereof by the method as herein described and administering to a subject with cancer said antibody or antigen-binding fragment thereof, or with an immune cell expressing said antibody or antigen-binding fragment thereof, in a therapeutically effective amount to treat said subject.

The present disclosure also relates to an antibody (including variants and derivatives thereof) in particular a TCR-like antibody, a T cell receptor (TCR) (including variants and derivatives thereof), or a CAR (including variants and derivatives thereof), in particular an MHC-restricted antibody-based chimeric antigen receptor, which are directed against a tumor neoantigenic peptide as herein described, for use in cancer therapy of a subject, wherein the tumor neoantigenic peptide binds at least one MHC molecule of said subject.

The present disclosure also relates to an antibody (including variants and derivatives thereof) in particular a TCR-like antibody, a T cell receptor (TCR) (including variants and derivatives thereof), or a CAR (including variants and derivatives thereof), in particular an MHC-restricted antibody-based chimeric antigen receptor, which are directed against a tumor neoantigenic peptide as herein described, optionally in association with an MHC or HLA molecule, or an immune cell which targets a neoantigenic peptide, as previously defined, for use in adoptive cell or CAR-T cell therapy in a subject, wherein the tumor neoantigenic peptide binds at least one MHC molecule of said subject. Typically, the skilled person is able to select an appropriate antigen receptor which binds and recognizes a tumor neoantigenic peptide as previously defined with which to redirect an immune cell to be used for use in cancer cell therapy. In a particular embodiment, the immune cell for use in the method of the present disclosure is a redirected T-cell, e.g. a redirected CD8+ and/or CD4+ T-cell.

In some embodiments, cancer treatment, vaccination therapy and/or adoptive cell cancer therapy as above described are administered in combination with additional cancer therapies. In particular, the T cell compositions according to the present disclosure may be administered in combination with checkpoint blockade therapy, co-stimulatory antibodies, chemotherapy and/or radiotherapy, targeted therapy or monoclonal antibody therapy.

Checkpoint inhibitors include, but are not limited to, PD-1 inhibitors, PD-L1 inhibitors, Lag-3 inhibitors, Tim-3 inhibitors, TIGIT inhibitors, BTLA inhibitors, V-domain Ig suppressor of T-cell activation (VISTA) inhibitors and CTLA-4 inhibitors, IDO inhibitors for example. Co-stimulatory antibodies deliver positive signals through immune-regulatory receptors including but not limited to ICOS, CD137, CD27 OX-40 and GITR. In a preferred embodiment the checkpoint inhibitor is a CTLA-4 inhibitor.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNa, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,ρ′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

‘In combination’ may refer to administration of the additional therapy before, at the same time as or after administration of the T cell composition according to the present disclosure.

In addition or as an alternative to the combination with checkpoint blockade, the T cell composition of the present disclosure may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells including but not limited to PD-1, Lag-3, Tim-3, TIGIT, BTLA CTLA-4 and combinations of these. The T cell as discussed here may be modified by any of these methods.

The T cell according to the present disclosure may also be genetically modified to express molecules increasing homing into tumours and or to deliver inflammatory mediators into the tumour microenvironment, including but not limited to cytokines, soluble immune-regulatory receptors and/or ligands.

In a particular embodiment, said tumor neoantigenic peptide is used in cancer vaccination therapy in combination with another immunotherapy such as immune checkpoint therapy, more particularly in combination with antibodies anti-PD1, anti-PDL1, anti-CTLA-4, anti-TIM-3, anti-LAG3, anti-GITR.

### BRIEF DESCRIPTION OF THE DRAWINGS

FIG. 1: Tumor neoantigenic peptides (or TE-derived epitopes) having a predicted affinity for MHC alleles of less than 500 nM, identified by the in silico method according to the disclosure in the tumor mouse lines B16F10-OVA cells (A) and in MCA101-OVA cells (B) and identified both in the two lines (C).

FIG. 2: (A) RT-PCR gels of amplification of the fusion transcript sequence encoding the neoantigenic peptide N25, in cDNA of tumor mouse lines B16F10-OVA and MCA101-OVA. (B) RT-PCR gels of amplification of the fusion transcript sequence encoding the neoantigenic peptide N26, in cDNA of tumor mouse lines B16F10, B16F10-OVA and MCA101-OVA.

FIG. 3: (A) Detection of peptide-reactive IFNg-secreting cells by ELISPOT in inguinal lymph nodes from immunized animals with DMSO (negative control), OVA (ovalbumine) (positive control), peptide N25 or peptide N26. (B) IFNg spots for 10\hat{ }5 cells for immunized animals with DMSO (negative control), SIINFEKL (positive control), N25 or N26 peptide.

FIG. 4: (A) Evolution of the tumor volume (mm3) in mice beforehand immunized with DMSO, OVA or N25L peptide, following the days after the injection of tumor cells B16F10-OVA into said immunized mice. (B) Evolution of the tumor volume (mm3) in mice beforehand immunized with DMSO, OVA or N26L peptide, following the days after the injection of tumor cells B16F10-OVA into said immunized mice.

FIG. 5: TCGA data sets for 784 luminal, 100 HER2+, 197 TNBC, 112 normal breast tissue, 516 primary lung adenocarcinomas (primary tumor) and 59 normal lung tissue (solid tissue normal), were analyzed by the method for identifying fusion transcript sequence encoded tumor neoantigenic peptide described. (A) Number of fusion transcript sequence (TE-exon fusions) in different subtypes of breast cancer (HER2+, TNBC, normal breast tissue and luminal). (B) Number of fusion transcript sequence (TE-exon fusions) in different subtypes of lung cancer (primary lung adenocarcinomas, normal lung tissue).

FIG. 6: 8-9 amino acid-long peptides predicted from TE-gene fusion products from each sample were tested in silico for binding to the predicted HLA alleles expressed in the same sample. Shown are peptides with predicted affinity below 500 nM for at least one HLA-A, -B, or -C allele from each sample. (A) Samples of different subtypes of breast cancer (HER2+, TNBC, normal breast tissue and luminal). (B) Samples of different subtypes of lung cancer (non-small cell lung cancer, normal lung tissue).

FIG. 7: Distribution of tumor-specific peptides per patient across breast tumor subtypes. (A) Numbers of tumor-specific HLA-binding peptides per subtypes of breast cancer patient are shown. (B) Numbers of predicted tumor neoantigenic peptides shared across luminal subtypes samples (n=784) (abscissa). (C) Numbers of predicted tumor neoantigenic peptides shared across HER2+ subtypes samples (n=100) (abscissa). (D) Numbers of predicted tumor neoantigenic peptides shared across TNBC subtypes samples (n=197) (abscissa).

FIG. 8: (A) Numbers of tumor-specific HLA-binding peptides per primary lung adenocarcinomas (LUAD) sample (lung cancer). (B) Distribution of tumor-specific peptides per patient across lung adenocarcinomas. Numbers of predicted tumor neoantigenic peptides shared across primary tumor subtypes samples (n=516) (abscissa).

FIG. 9: Reconstruction of the fusion nucleotide sequence when the donor is the exon (A) and when the donor is the TE (B).

FIG. 10: Binding of chimeric transcripts-derived peptides to HLA-A2. Binding to HLA-A2 allele of predicted peptides from the most frequent chimeric fusions were validated by flow cytometry using tetramer formation assay. The results are shown as percentage of binding relative to positive control. Dotted line indicates the threshold considered to confirm the binding to this allele.

FIG. 11. Immunogenicity of fusion transcripts-derived peptides and reactive CD8+ T cells generation. (A) Frequencies of pJET (fusion transcript derived peptides) specific tetramer-positive CD8+ T cells expanded from 6 different healthy donors in in vitro immunogenicity assays using 6 different healthy donors. (B) Cytokine secretion of CTL-clones after stimulation with different concentration of specific peptide. On the right is listed the CTL-clones generated and their peptide specificity. (C) Killing assay for CTL-clone 9 in co-culture with target cells loaded with 2 different peptide concentration in combination with anti-MHC-I antibodies or Isotype control (Left panel), or with un-loaded targets cells at different ratios (Right panel). (D) Killing assays for CTL-clone 9, 80 and 64 when co-cultured with peptide unloaded target cells in combination with anti-MHCI-I antibodies or isotype control. Effector: Target ratio is indicated in each individual plot. H1650 were used as target cells for each plot of this figure.

FIG. 12. Expression of TCR recognizing fusion-derived peptides. Transduced Jurkat-reporter cells with TCR sequence derived from CTL-clone 9 co-cultured with target cells alone, or loaded with 2 different peptide concentration. Plots show percentage of positive Jurkat cells for the 3 reporter genes evaluated by flow cytometry, using H1650 cell line as target cells (upper plots) or H1395 cell line as target cells (lower plots). Negative control: non-transduced Jurkat cells. No peptide: transduced Jurkat cells co-cultured with peptide unloaded target cells. Positive control: Transduced Jurkat cells stimulated with PMA/ionomycin.

FIG. 13. Tumor infiltrating lymphocytes recognizing fusion transcripts-derived peptides. Percentage of tetramer positive CD8 T cells for the indicated fusion transcript-derived peptides found in tumor infiltrating lymphocytes (TILs) expanded in the presence of fusion transcripts-derived peptide's mix+IL2 (A) or only with IL-2 (B).

FIG. 14. Phenotype of CD8+ T cells recognizing fusion transcripts-derived peptides in LUAD patient's derived samples. Percentage of tetramer positive CD8 T cells recognizing fusion transcripts-derived peptides present in tumor, juxta tumor, lymph nodes and blood samples derived from LUAD Patient 2 (A, upper panel) and Patient 3 (B, upper panel). In lower panel of figure (A) and (B) is shown the percentage of Naïve (CCR7+CD45+), Central Memory (CM, CCR7+CD45−), Effector Memory (EM, CCR7−CD45−) and Terminal Effector (TE, CCR7−CD45+) cells of tetramer positive parental cell population.

FIG. 15. Immunopeptidomics analysis of lung tumor samples. Fusion transcript-derived peptide sequences were searched in public MHC-I immunopeptidomes datasets. Each column represents a different sample. Each row represents a different peptide sequence (specify on the right). Colored squares indicate in which sample is found each fusion transcript-derived peptide. Publications describing each sample data-sets are annotated on the top.

FIG. 16. Immunopeptidomics. A. Identification of JET derived peptides across 17 primary lung tumors and the human adenocarcinoma cell line A549, treated or not, with interferon gamma. Peptides are shown in rows and samples in columns. B. Boxplot showing the comparison of MS/MS scores obtained from the annotated peptidome (canonical peptides) and from the pJET peptidome. C. Comparison of the frequencies of peptides identified from the annotated peptidome (canonical peptides) and the pJET peptidome at different amino acid peptide lengths.

FIG. 17. Binding of ER-derived peptides to HLA-A2 molecule. Peptides-HLA-A*02:01 complex formation for synthesized chimeric transcripts-derived peptides. Percentage of complex formation relative to positive control (CMV pp65 495-503) is represented. The mutated (MelA Mut) and non-mutated (MelA) sequences of Melan-A were used as strong and weak binder peptides controls, respectively. ‘Negative’ indicates staining background. Dashed line indicates the minimum complex formation value needed to consider a peptide as good binder to HLA-A*0201 (50% of positive control).

FIG. 18. A. Activation of Jurkat cells transduced with CTL-clones-derived TCRs recognizing chimeric transcripts-derived peptides after co-culture with target cells loaded with relevant/specific or an irrelevant/unrelated peptide (Melan-A). B. Activation of Jurkat cells transduced with CTL-clones-derived TCRs recognizing chimeric transcripts-derived peptides after co-culture with target cells loaded with relevant peptide or unrelated peptide (Melan-A), in presence or absence of anti-MHC-I blocking antibody (W6/32) or isotype control. PMA/Ionomycin was used as a positive control of activation and target cells without loading peptides were used as negative control of activation. H1395 LUAD cell line were used as target cells. CTL-clone from which each TCR is derived is indicated on the top and peptide specificity between brackets, showing aminoacidic sequence of chimeric transcript-derived peptide recognized by each of these TCR. This peptide sequence is the specific/relevant peptide used in each case to load target cells. Melan-A and MelA Mut both refer to the unrelated peptide (ELAGIGILTV).

FIG. 19: A. Heatmap summarizing the frequency of CD8+ T cells recognizing chimeric transcript-derived peptides found ex-vivo without T cell expansions. Only peptide specificities found in at least one tissue are shown (total evaluated patients=4). B. CCR7 and CD45RA percentages in tetramer positive cells summarized in A. after ex-vivo staining for patient 2 and patient 5. (no data available for Patient 1). C. Heatmap summarizing specific tetramer positive cells recognizing chimeric transcripts-derived peptides after in-vitro expansions at day 20 on CD8+ T cells from tumor, juxta tumor or tumor-draining LN samples in the 5 patients analyzed. Only peptide specificities found in at least one tissue are shown. Black squares highlight peptide specificities found also ex-vivo in the same tissue and patient.

### EXAMPLES

### 1. Example 1: Identification of Fusion Transcript Sequence Encoded Tumor Neoantigenic Peptide

### 1.1 Proof of Concept in Mice

To detect individual and shared tumor neoantigenic peptide issued from fusion transcripts sequences, a bioinformatics pipeline has been developed. This pipeline is designed to identify tumor-specific mRNA sequences composed in part of a TE sequence and in part of an exonic sequence. This pipeline implies determining the MHC alleles. For each human sample, the Class I and Class II MHC alleles can be determined using the seq2hla (v2.2) tool (bitbucket.org/sebastian_boegel/seq2hla). For mouse models, murine H-2 alleles are generally known. The bioinformatics method comprises the mapping of transcripts from RNA-sequencing against the reference genome. For the proof of concept analyses described here, mm10 was used for mouse and hg19 for human. Different versions of assembled genomes can be used for example hg19, hg38, mm9 or mm10. This mapping is carried out with STAR (v2.5.3a) (github.com/alexdobin/STAR), with the following setting:
For allowing multi-hits mapping the parameter outFilterMultimapNmax which sets the maximum number of loci, the read is allowed to map to, is set at 1000, and
For detecting the abnormal junction (fusion), the parameter chimSegmentMin which sets the minimum length of fusion segment, is set at 10, the parameter chimJunctionOverhangMin which sets the minimum overhang for a fusion junction is set at 10.

Normal (from SJ.out.tab output file) and abnormal (from Chimeric.out.junction output file) junctions are annotated using Ensembl and repeatmasker databases. Normal junctions define all the junctions that match the parameters used for the mapping (maximum intron length<=1 000 000 bp (set by --alignIntronMax), same chromosome and well oriented) and abnormal ones are junctions that do not match with at least one of the previous criteria. This mean that a TE/Exon junction could be in both junction type but a Exon/Exon junction must be in normal file (SJ.out.tab). Transcript sequences comprising a junction between a TE sequence and an exonic sequence are extracted in silico. From the area of the transcript sequence which overlaps the junction, or downstream of the junction when out-of-frame (reading frame non-canonical), the software predicts, in all reading frames, all possible peptides of 8 or 9 mers. Then, the binding affinity of all these possible peptides for the MHC alleles previously defined for the matched sample is determined netMHCpan (v3.4) (cbs.dtu.dk/services/NetMHCpan/). There are currently more than a dozen various prediction algorithms for predicting the binding affinity of peptides, with NetMHC being the most widely used and validated algorithm for neoantigen prediction pipelines.

Peptides with either less than 500 nM or with a percentile rank less than 2% are considered as potential neo-antigens. Each splice site (donor or acceptor) is uniquely annotated as TE or as Exon. The part in the 5′ end is qualified “donor”, and the part in the 3′ is qualified “acceptor”. Predicted HLA-binding peptides shared between cancer and normal tissues are excluded from further analyses.

This method has been applied to RNAseq data obtained from 7 well-characterized murine tumor cell lines (B16F10, B16F10-OVA, MCA101, MCA101-OVA, MC38, MC38-GFP, MC38-GFP-OVA). The cell lines with the extension-OVA corresponding to the same model but further expressing ovalbumin. In this study, this line is considered as the similar model, that is to say for example that an assay carried out on the cell line from B16F10-OVA is considered as a repeat of an assay carried out on the cell line from B16F10.

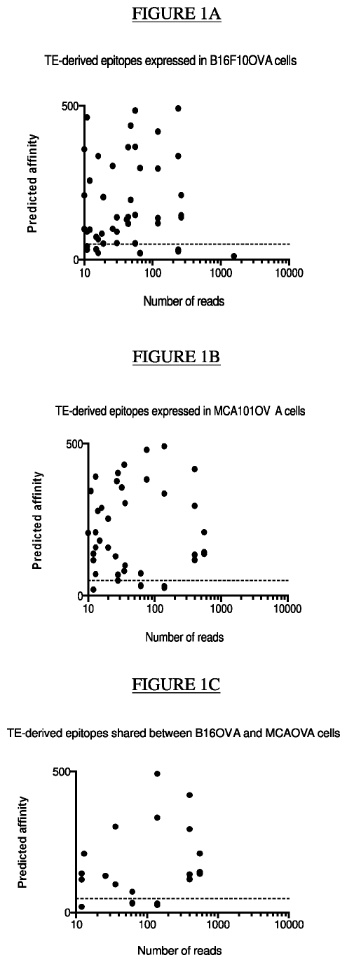

A list of candidate peptides has been obtained with these parameters (FIGS. 1A, 1B and 1C), some were specific to particular cell lines (FIGS. 1A and 1B), and some were shared between the two tumor cell lines (FIG. 1C).

For validation, we selected a range of peptides, expressed either in B16F10-OVA or MCA101-OVA, with predicted affinities less than 500 nM. Peptides were selected trying to optimize the ratio between number of reads and predicted affinity for MHC-I.

Four predicted tumor neoantigenic peptides were selected and characterized by identifying the TE and the exonic sequence (table 2).

TABLE 2
Characterization of 4 predicted tumor neoantigenic peptides selected by the method

| Peptide | Cell line | Donor | Acceptor | Predicted affinity |
|---|---|---|---|---|
| N25 | B16/B16-OVA | ERV-MaLR | Chmp3, exon2 | H2-Db, 51.8937 |
| | | (subfamily | | |
| | | MTA) | | |
| N26 | MCA/MCA-OVA | SINE- | Angel2, exon2 | H2-Kb, 392.0384 |
| | MC38-GFP/MC38-OVAGFP | Alu(B1F) | | |
| N90 | MCA/MCA-OVA | Predicted gene | ERVL-MaLR | H2-Kb, 403.8959 |
| | | 45873 | (subfamily | and 50.5416 |
| | | | ORR1A2-int) | |
| N94 | MCA/MCA-OVA | Rsrc1 | ERV1 (subfamily | H2-Kb, 431.0564 |
| | MC38-GFP/MC38-OVAGFP | | RLTR4_MM-int) | |

### 1.2 Validation by RT-PCR of the Fusion Transcript Sequence

First, a validation by regular RT-PCR has been performed, using primer pairs with one primer in the TE sequence, and the other one in the exonic sequence.

For the RNA extraction and reverse transcription, 3-5.10⁶ cells were lyzed in 500 μL Trizol, and 100 μL phenol-chloroform added to the lyzates prior centrifugation. Aqueous phase was collected, mixed in a 1:1 ratio with 100% EtOH and transferred to RNAeasy minikit columns. RNA was then collected following manufacturer's instructions (including on column DNAse treatment). After RNA elution, DNA contaminants were further removed by treatment with Turbo DNAse (Fisher scientific), according to manufacturer's instructions). RNA concentration was measured using a nanodrop, and 1 μg of RNA used for reverse transcription. First strand synthesis was performed with Superscript III (Life technologies) using oligodT (15) as primers, according to manufacturer's instructions. Primers were ordered from Eurogentec. PCR reactions were performed using Taq polymerase. After identification of optimal conditions for each reaction, PCR products were extracted from agarose gels, and sequencing was performed using GATC lightrun. Sequence alignment was checked with APE software.

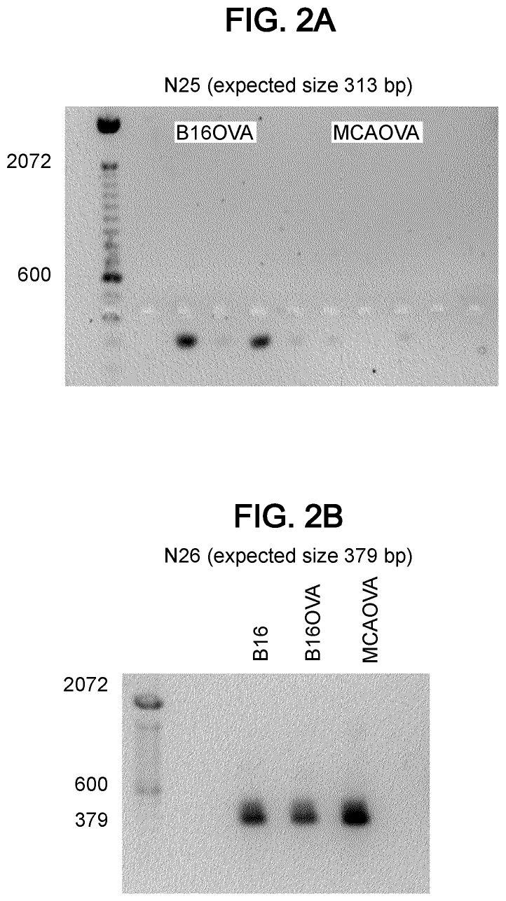

Using this approach, bands matching predicted size for N25, N26, N90 and N94 were detected, respectively in the cell lines identified in Table 2 (See FIG. 2A for N25). Interestingly, although N26 was detected only in MCA and MC38 cells in silico by RNAseq as previously described in the pipeline, using RT-PCR we detected a band corresponding to N26 in B16F10-OVA cells (FIG. 2B), indicating that this sequence is shared between three independent tumor cell lines (MCA, MC38 and B16F10). By re-analyzing the RNAseq data, we found that the N26 junction was present in B16F10-OVA cells, but below the detection threshold of the algorithm. Moreover, sequencing of the RT-PCR product showed exact match with sequences predicted by the algorithm.

### 1.3 In Vivo Immunization of Mice

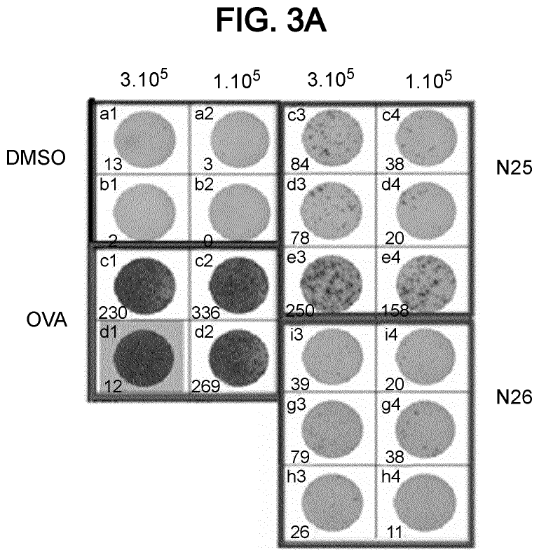

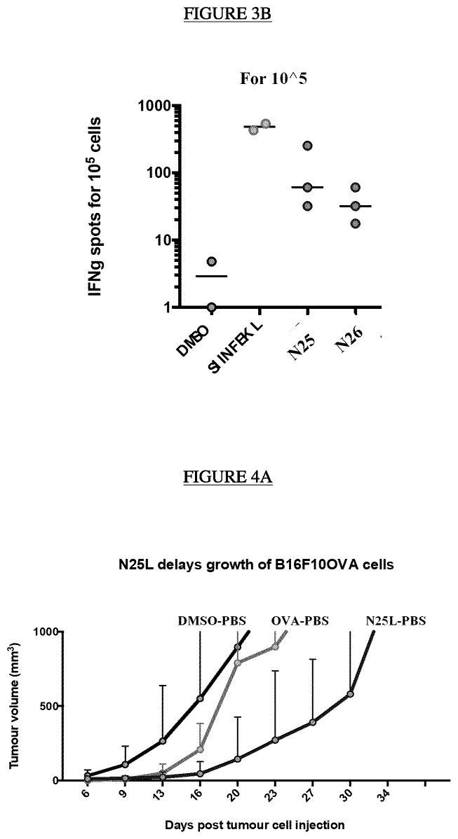

To validate these candidates in vivo, short (9-mers) peptides corresponding to neoantigenic peptide which binds to the MHC class I sequences, were synthetized. For the in vivo assays, long (27-mers) peptides, which include the flanking regions to the predicted MHC-binding short peptides of 9 mers, were synthetized, because this length is better suited for in vivo immunization. B16F10 OVA and MCA101-OVA were maintained in RPMI, Glutamax, 10% FCS, 1% penicillin-streptomycin and passaged using TrypLE. Cells were kept in culture for a maximum of one month, and new vials were thawed for each in vivo experiment. C57BL6J recipient mice were immunized with 100 μg long peptide (N25L or N26L), SIINFEKL peptide (short OVA peptide), OVA (Sigma) or DMSO, each with 50 μg polyI:C, by subcutaneous injection into the flank. Immunizations were repeated 7 days after primary immunization. 3 days later (10 days after primary immunization), animals were sacrificed and numbers of peptide-specific IFNg-secreting CD8 T cells in inguinal lymph nodes were detected by ELISPOT (FIG. 3A). Short peptides (N25, N26, or SIINFEKL) or DMSO at 10 μg·mL⁻¹ were used to restimulate T cells. Alternatively, 7 days after secondary immunization, animals were injected subcutaneously with 2.5.10⁵ B16F10-OVA or 5.10⁵ MCA-OVA cells in PBS. We found that N25, and to a lesser extent N26 were able to induce immune responses (FIG. 3B).

1.4 In Vivo Treatment of Mice with Tumor

To test whether these peptides were protective against tumor cells, we immunized C57BL6 mice with 100 mg peptides N25L or N26L, or OVA (control peptide) and 50 μg polyI: C in PBS at d0 and d7, and at d14, we injected 2.5.10⁵B16F10-OVA cells to mice immunized with OVA, N25L and N26L. B16F10 OVA and MCA101-OVA were maintained in RPMI, Glutamax, 10% FCS, 1% penicillin-streptomycin and passaged using TrypLE. Cells were kept in culture for a maximum of one month, and new vials were thawed for each in vivo experiment. C57BL6J recipient mice were immunized with 100 μg long peptide (N25L or N26L), OVA (Sigma) or DMSO, each with 50 μg polyI: C, by subcutaneous injection into the flank. Immunizations were repeated 7 days after primary immunization.

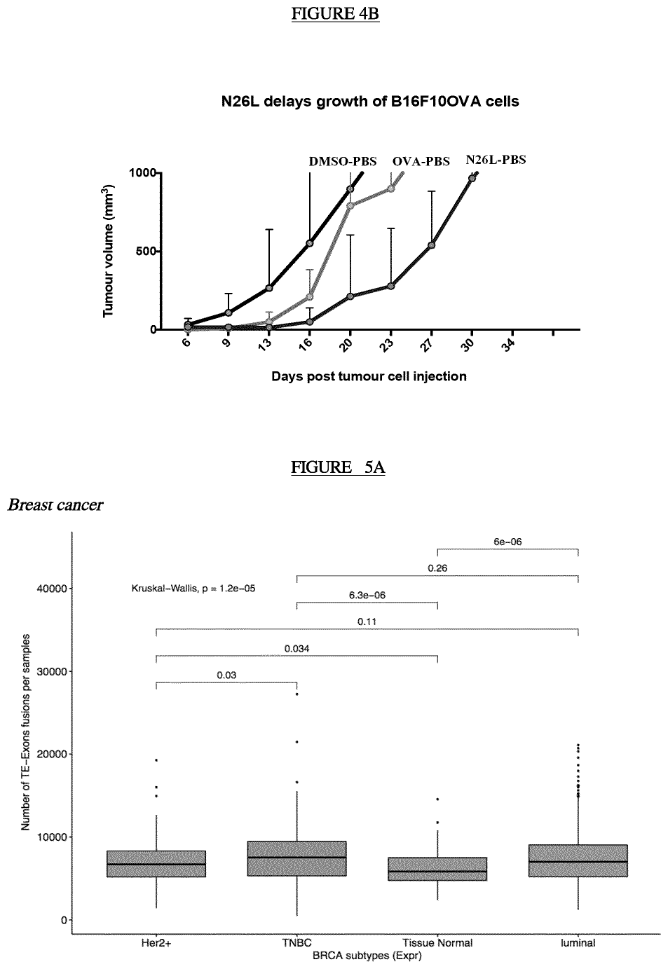

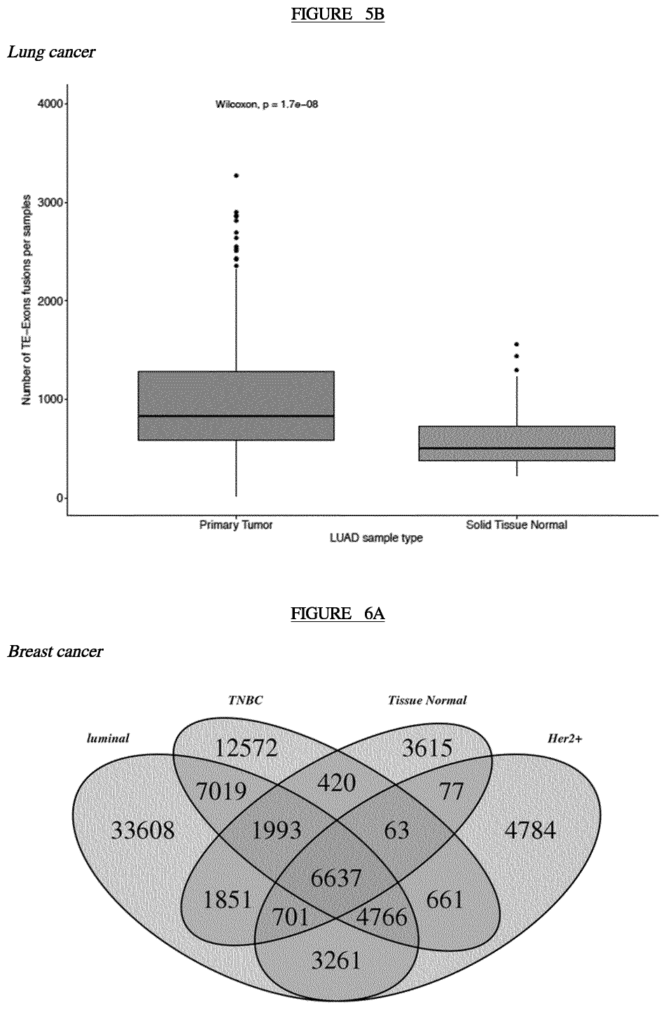

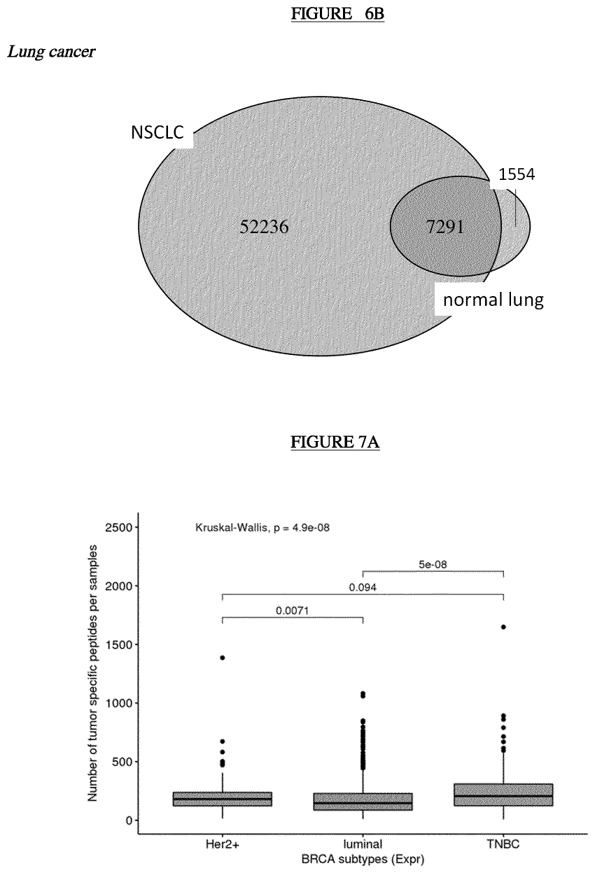

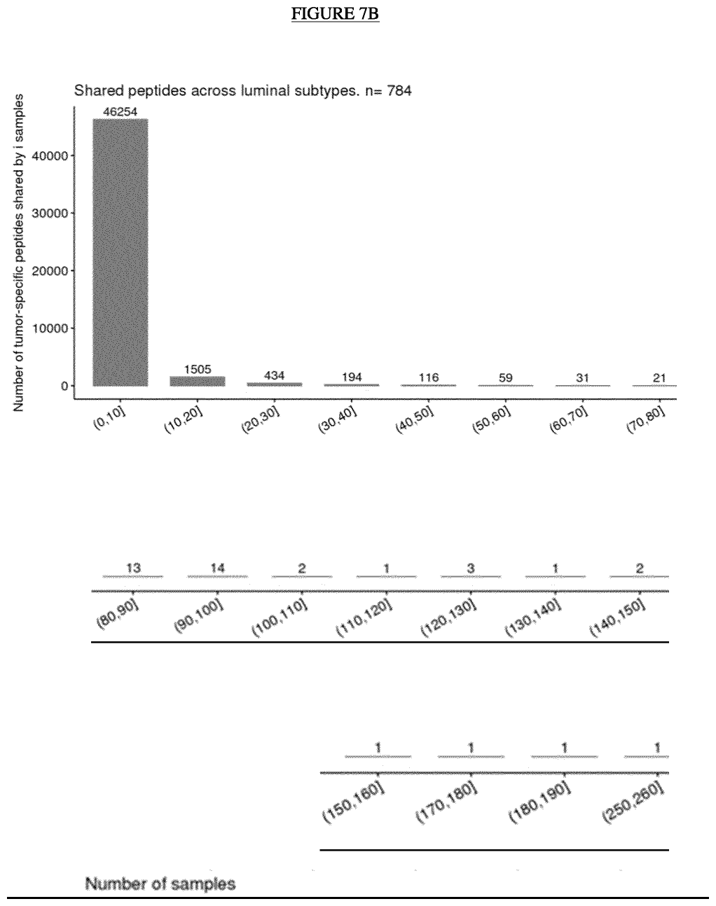

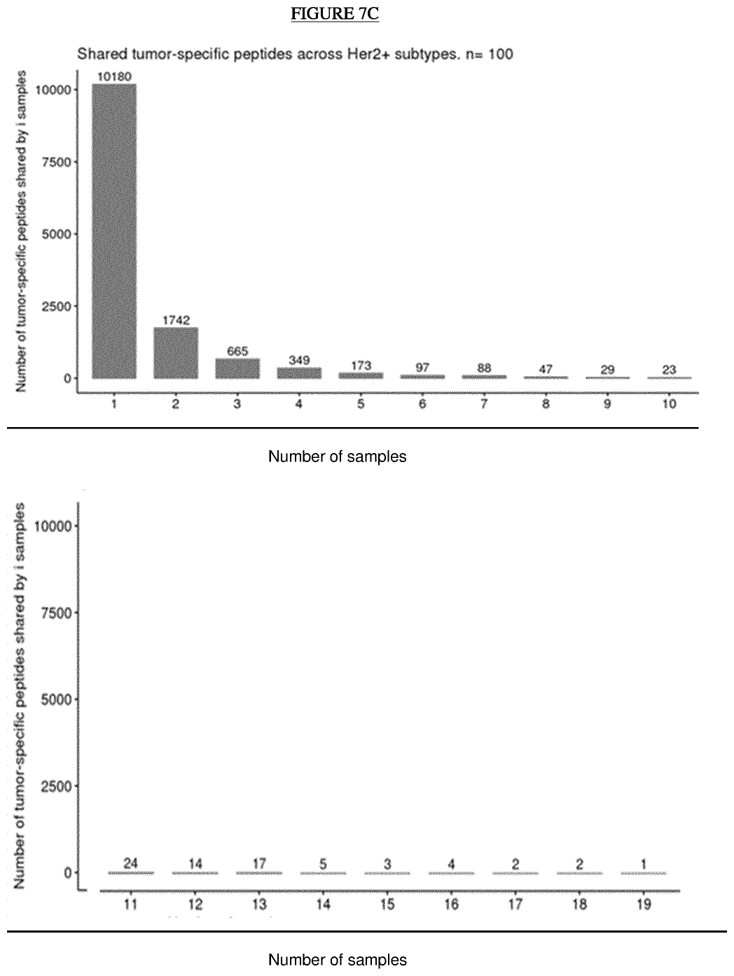

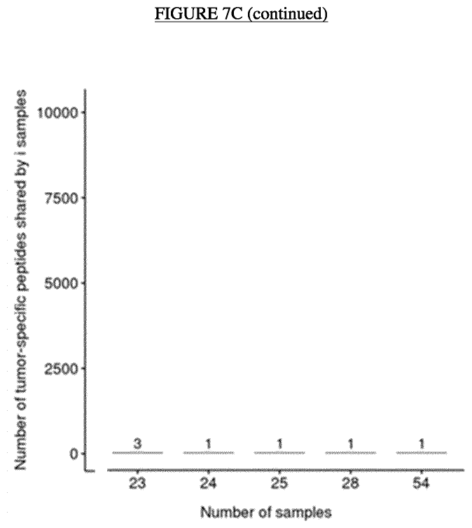

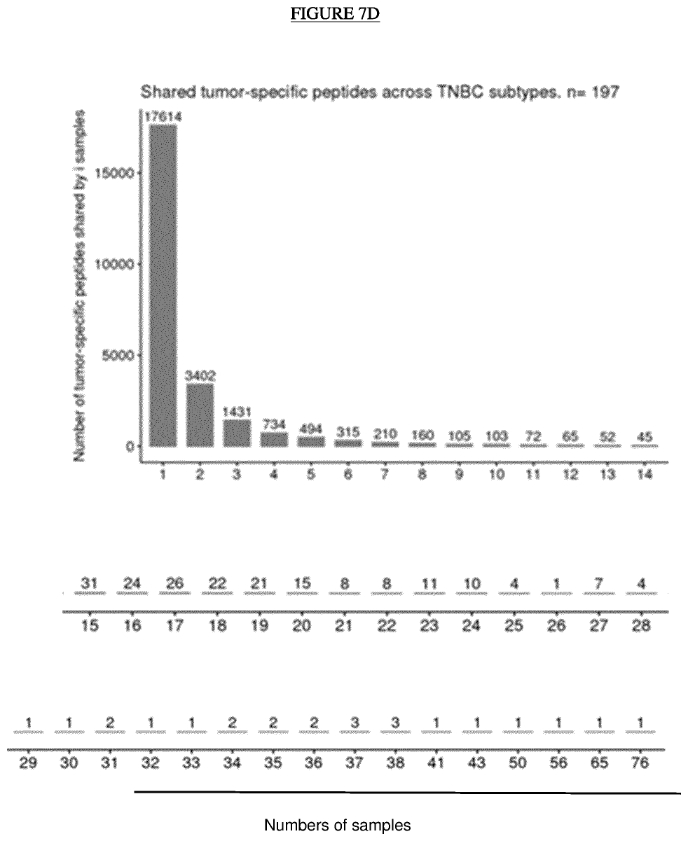

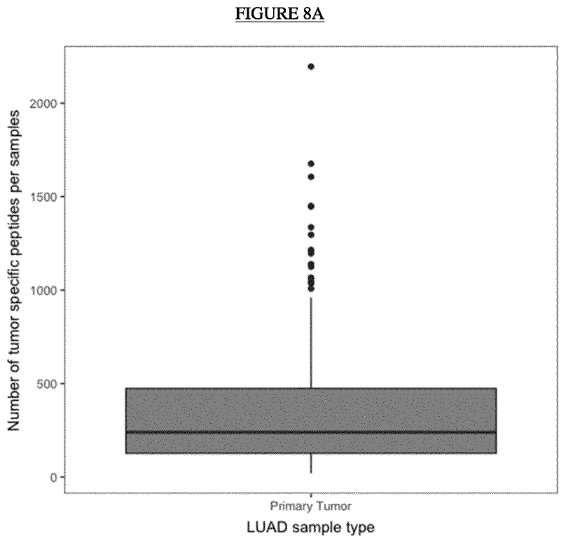

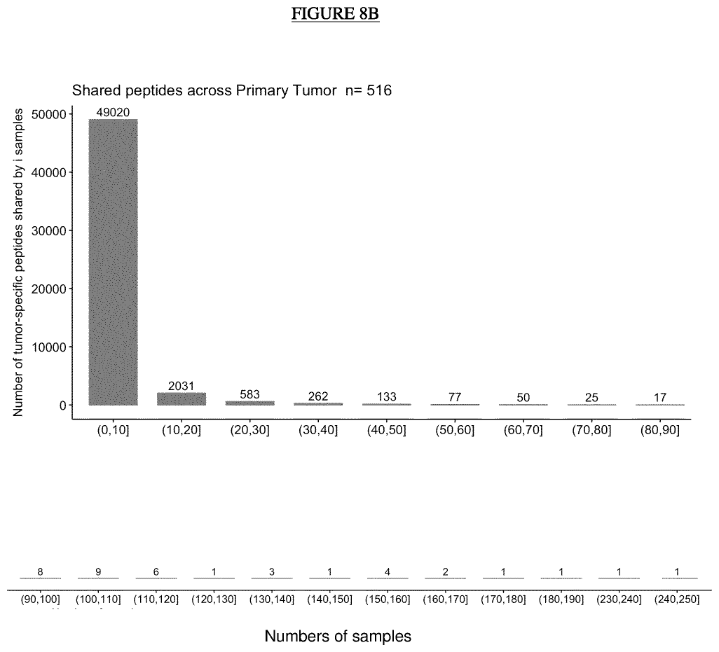

Short peptides (N25, N26, or SIINFEKL) or DMSO at 10 μg·mL⁻¹ were used to restimulate T cells. Alternatively, 7 days after secondary immunization, animals were injected subcutaneously with 2.5.10⁵ B16F10-OVA or 5.10⁵ MCA-OVA cells in PBS. Tumor size was measured twice weekly using a manual caliper, and animal health status monitored throughout the experiment timeframe (FIGS. 4A and 4B). Animals were sacrificed when tumor volume reached 1 mm³. Strikingly, we observed that N25L significantly delayed the formation of B16OVA tumors, in a more efficient way than OVA. Moreover, we obtained a similar result upon N26L immunization.

### 2 Example 2: Identification of Human Lung Adenocarcinoma (LUAD) Neoantigenic Peptides Derived from Fusion Transcripts Composed of a TE Element and an Exonic Sequence

### 2.1 Material and Methods

RNA extraction. Tumour and juxtatumour samples were cut into pieces of #1 mm³ and resuspended in 700 μl RTL lysis buffer (Quiagen) supplemented with 1% β-mercaptoethanol and homogenized using Perecellys 24 Tissue Homogenizer (Bertin Technologies). Total RNA isolation was performed using RNeasy Micro Kit (Qiagen) following manufacturer instructions. Total RNA from tumour cell lines were extracted from 5.10⁶ tumor cell lines using the same procedure.

PCR and Sequencing. Primers were designed using APE software. For each sample, 1 μg of RNA was retrotranscribed into cDNA using SuperScript III Reverse transcriptase (ThermoFisher), as indicated by the provider. PCR reaction was performed using GoTaq G2 Hot Start Polymarase (Promega). All primers were used in a concentration of 0.5 μM. Reactions were carried out in Veriti™ 96-Well Thermal Cycler (ThermoFisher). PCR products were loaded in LabChip GX (Caliper LifeSciences) and analysed by LabChip GX Software (v4.2).

PCR reactions were repeated for those samples with an amplification product on the expected size. Then, the PCR products were run in a 2% agarose gel SYBR Free Dye (1/10000) (Invitrogen). The specific bands were cut and the DNA products were purified using QIAquick Gel Extraction Kit (Qiagen) following manufacturer instructions. Finally, these products were sequenced by EuroFins Scientific. The resulting sequences were compared to the expected one using Serial Cloner software.

Tetramer formation. HLA-A2 monomers were purchased from ImmunAware® and the formation of tetramers was evaluated with synthetic ER-derived peptides following manufacturer instructions. Briefly, synthetic HLA-A2 monomers were incubated with synthetic peptides during 48h at 18° C. Tetramerization was done by further incubation of monomers with biotinylated-sepharose. Finally, tetramer formation was measured by flow cytometry using a PE-conjugated anti-β2-microglobulin antibody. As a positive control we used a peptide derived from CMV provided by the manufacturer.

In experiments addressed to evaluate the presence of specific CD8+ T cells, the tetramerization step was performed by incubating the monomers with different combinations of fluorescent streptavidin (PE, APC, PE-Cy5, PE-CF594, BV421, BV711 and FITC).

Priming of naïve CTLs. PBMCs were obtained by Ficoll gradient separation from HLA-A2+ healthy blood donors. CD14+, CD4+ and CD8+ cells were purified by positive selection using magnetic beads (Miltenyi Biotec). While CD4+ and CD8+ T cells were cryopreserved until the experiment day, CD14+ fraction was cultured in the presence of IL-4 (50 ng/ml) and GM-CSF (10 ng/mL) at 106 cells/mL during 5 days to obtain moDCs. After this period of time, the moDCs were maturated with LPS and incubated with synthetic ER-derived peptides at a final concentration of 1 μg/mL for 2 hours. Finally, peptide-loaded moDCs were co-cultured with autologous CD4+ and CD8+ T cells in culture medium supplemented with with IL-2 (10U/ml) and IL-7 (100 ng/ml). The ER-derived peptide stimulation of specific CD8+ CTL populations was assessed by MHC-I tetramer staining by flow cytometry using a combination of two-color tetramer for each peptide.

Tetramer Staining. Cells were resuspended in PBS, stained with Live/Dead Aqua-405 nm (ThermoFisher) during 20 minutes at 4° C. and washed once. After that, cells were resuspended in PBS-1% BSA containing the mix of SA-coupled tetramers and incubated in the dark at room temperature during 20 minutes. Without further washing, surface antibodies were added in PBS-1% BSA and cells were incubated 20 minutes in the dark at 4° C. Surface antibodies were a combination of anti-CD3-BV650+ anti-CD8-PECy7 in combination with anti-CCR7-AF700+ anti-CD45RA-BUV395 when required. Finally, cells were washed twice and resuspended in FACS buffer for flow cytometry analysis.

CTL-clones generation. Tetramer positive cells were single-cell FACS sorted (ARIA-sorter, BD) in U bottom 96-well plates. Sorted cells were collected in 100 μl of RPMI 10% human serum AB (Sigma-Aldrich) containing 150.000 feeders' cells. Finally, 100 μl of AIM-medium containing IL-2 (3000 IU/ml) and anti-CD3 (100 μg/ml, OKT3 clone from Miltenyi) were added and cells were cultured during 15-20 days maximum. When evident cell growth was observed in wells, we perform a second round of expansions with new feeders' cells for an additional period of 15 days maximum. Cells were feed and split as necessary during this period with the same culture media (AIM-RPMI 50/50+5% Human Serum) but only containing IL-2 at 500 IU/ml. Finally, expanded clones were checked for their specificity by FACs-tetramer staining and only clones with >85% of tetramer positive clones were used for further analysis.

Killing assays. To perform killing assays, xCELLigence RTCA S16 Real Time Cell Analyzer was used. H1650 cell-line were plated at 0.5×10⁶ cells/ml in pre-coated 16 well plates. One day after, cells were incubated or not during 1 h with different concentration of the correspondent synthetic peptides. After that, cells were washed twice with culture medium and incubated or not for additional 30 minutes with anti-MHC-I antibodies (clone W6/32, 50 μg/well) or isotype control at the same concentration. Without additional wash, CTL-clones were added at the correspondent ratio. The complete assay was done in free-serum culture medium in a final volume of 200 at 37° C. connected to the xCELLigence system. Impedance variation (cell-index) was measured in real-time during 40 h. Each condition was performed by duplicates.

Cytokine secretion and Jurkat cells activation. 50.000 H1650 cells were plated in 96-well plate in culture medium supplemented with 5% of fetal bovine serum. The day after, cells were culture during 1-2 h with synthetic peptides at different final concentrations. After that, cells were washed twice, CTL-clones were added at 1:1 ratio and co-cultured during 18 h with peptide-loaded target cells. Culture supernatants were collected and cytokine concentration analyzed by cytokine beads arrays (CBA, BD Biosciences) following manufacturer's instructions.

The same experiment was performed using transduced Jurkat cells instead of CTL-clones and two different types of target cells: H1650 and H1395 cell lines. In this assay, after co-cultured with peptide-loaded target cells, Jurkat cells were assessed by flow cytometry analyzing the expression of reporter markers. PMA/Ionomycin was used as positive control to activate Jurkat cells.

Tissues and Blood samples. Lung tumor, juxta tumor and lymph nodes samples were cut into small pieces and digested using a mix of collagenase-I (2 mg/ml), hyaluronidase (2 mg/ml) and DNasa (25 μg/ml) in a final volume of 2 ml culture medium (CO₂ independent medium+5) during 40 min at 37° C. After digestion single cell suspensions were collected through a cell Strainer and washed. Tumor and Juxta tumor suspensions were enriched on lymphocyte fractions by a ficoll gradient. After that cells were staining for tetramer analysis by FACs as described before.

Blood samples were seeded on a ficoll gradient and PBMCs were isolated. After that, PBMCs were enriched for CD8+ T cells using EasyStep Human CD8+ T cell Enrichment Kit (STEMCELL Technologies). Finally, enriched cells were stained for tetramer analysis as described before.

Tumor infiltrating lymphocytes (TILs) cultures. Tumor tissue was cut into small pieces (1-3 mm³ size, 6-12 pieces maximum). Each tumor fragment was transferred into individual wells from 24-well plates and cultured in a final volume of 2 ml RPMI 10% Human Serum+IL-2 6000 IU/ml. Cells were feed/split as necessary during 15-20 days and cryopreserve or analyzed for tetramer staining.

TCR cloning. Total RNA was extracted from CTL-clones and retrotranscribed into cDNA using SuperScript III (ThermoFisher). TCRα and β were amplified by PCR as described in Li et al 2019. DNA products were run in 2% agarose gels and sequenced after gel band extraction (Qiagen). TCR V regions (a and B) were concatenated with murine TCR constant chain and cloned into a PEW-pEF1A-inactEGFP vector and amplified in transformed bacteria.

Jurkat transduction. Lentivirus particles were produced by HEK-293 FT cell line transfected with TCR-expression plasmids together with envelope (pVSVG) and packaging (psPAX2) plasmids. After 64 h, supernatant was collected and lentivirus particles were concentrated using 100 kDa centrifugal filter (Sigma-Aldrich). Lentivirus suspension was transferred by spinoculation into TCR-negative Jurkat cells expressing reporter genes (NFAT-GPF, NF-KB-CFP and AP-1-mCherry). After 5 days, transduction efficiency was evaluated by FACS using anti-murine TCR-B antibody (Clone H57-597). This Jurkat cells were described in Rosskopf S. et al. 2018.

Mass spectrometry data analysis. Public immunopeptidomics raw data derived from MHC-eluted peptides were analysed using ProteomeDiscoverer 1.4 (ThermoFisher) with the following parameters: no-enzyme, peptide length 8-15 aa, precursor mass tolerance 20 ppm and fragment mass tolerance 0.02 Da. Methionine was enabled as variable modification and a false discovery rate (FDR) of 1% was applied. MS/MS spectra were searched against the human proteome from Uniprot/SwissProt (updated 6 Mar. 2020) concatenated with the list of all fusion transcripts-derived proteins from lung TCGA projects. Finally, peptides matching with Uniprot database or with translated fusion transcripts present in lung normal samples were discarded.

### 2.2 Results: Identification of Fusion Transcript Sequences Encoding Tumor Neoantigenic Peptide in Human Subject

### 2.2.1 Characterization of Neoantigens

First the TE-Exon fusion transcript landscape was characterized in normal samples from TCGA public database. A total of 8876 unique fusions were identified in 679 normal samples from 19 different tissues (bile duct, bladder, brain, breast, cervical, colon, head and neck, kidneys, liver, pancreas, PCPG, prostate, rectum, sarcoma, skin, thymus, thyroid, uterine). Specific fusions to each tissue type were found with a very small portion of pan-tissue fusion transcripts. These results suggest that a dedicated tissue specific regulatory mechanism is associated with these fusion transcripts.

Then the number of identified fusions in 514 LUAD samples from TCGA has been compared to their 59 normal associated pulmonary samples present in TCGA. On average, 235 fusions were identified in NSCLC samples, compared with 200 in healthy lung samples (Wilcoxon pvalue=9×10.⁻¹⁰). 8269 total unique fusions were identified in NSCLC tumors.

A first category of fusions called TSF (tumor specific fusion) was obtained as those found in at least 1% of tumor samples and in none of the normal samples. 210 fusions were thus defined as TSF.

Some high-frequency fusion transcripts in tumors and low frequency in normal cells may also be good candidates for neo-antigens. Thus, a second category called TAF (tumor associated fusion) was notably defined as fusions present in less than 4% of normal tissues, notably less than 2%, and more than 10% of the tumors and that is over expressed in tumors compared to normal tissue samples.

Fusion sequence:
In order to reconstruct the fusion nucleotide sequence, the sequence of the donor on chromosome “Donor Chromosome X” from “Donor_start_X” to “Donor_Breakpoint_X” on strand “Donor_strand_X” and the acceptor sequence on the chromosome “Acceptor_Chromosome_X” starting from “Acceptor_Breakpoint_X” to “Acceptor_end_X” on the strand “Acceptor_strand_X” have been extracted from the Ensembl HG19 human assembly database. It is to be noted that the use of the Ensembl HG19 human database is not limitative and that any other adapted database may be used such as NCBI reference Sequence Database (RefSeq).
Care should be taken to take the reverse complement of the sequence if the fusion is present on the minus strand.
The “fusion sequence” consists of the donor sequence followed by the acceptor sequence.

Nucleotide sequence of the fusion transcript:

On the basis of the known canonical transcripts in which the exon is involved, all the “fusion transcripts” were reconstructed.

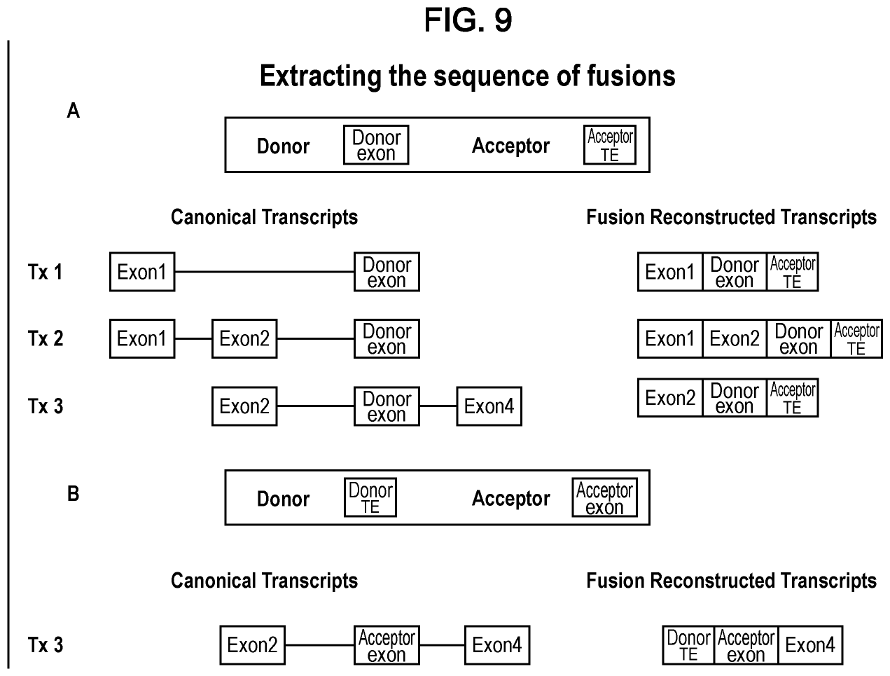

When the donor is the exon (see FIG. 9A)
it starts with the beginning of the canonical transcript to the donor exon and replace the complete canonical exon sequence with the fusion sequence. In this case, the fusion transcript stops after the TE sequence of the acceptor.

When the donor is the TE (FIG. 9 B)
The sequence begins at the canonical position of the acceptor exon in the transcript and forget all exons upstream. The canonical sequence of the acceptor exon was replaced with the fusion sequence and the transcript was reconstructed until the end.

Each nucleotide sequence of size k (i.e. from 24 to 75 nucleotides) of the fusion transcript (translation of the first k-mer starts at the first nucleotide of the fusion transcript, translation of the second k-mer starts at the second nucleotide of the fusion transcript, etc.) was then translated into a peptide sequence.

The obtained peptides are then further analyzed with NetMHCpan for MHC binding prediction. Affinity for binding to at least one of the known human alleles was thus predicted, (see also example 1 for further illustration) for each k-mer present in the sequence.

The peptides were then further screened against a reference proteome, typically for human subject against all sequences present in Uniprot (representing all the sequences encoded in the human exome). Peptides were considered equal to those in Uniprot if they had the same amino acid sequence or if they only differed in the amino acid in the first or last position. All these equal sequences were then discarded from the candidate list. 117 peptide sequences derived from these 230 fusion transcripts where thus predicted to bind to HLA-A2: 01 (see table 3 below).

TABLE 3
Peptides LUAD

| Peptide | Peptide | Peptide |
|---|---|---|
| sequence | sequence | sequence |
| RLLHLESFL | TLGGLMPVL | LMTSSIMSV |
| TLMNLVQVL | FLQGSITFI | MLMKTVWQA |
| ILHSLVTGV | MLLLYIWQV | SLQPEDMAL |
| FMMEQVGLA | YLKIMPVHL | KILTYFPMV |
| AMDGKELSL | HTLGGLMPV | FLGTRVTRV |
| TLAYGKYYI | YIMARVLFV | SLMQSGSPV |
| GLIQLIWLA | FILRTDHYI | VLMWTMAHL |
| GMVDGGSNI | IMSSAIAYL | LLGETKVYV |
| YLWTTFFPL | FIIGILQLA | KILTYFPMV |
| ALWEAKMII | YLLQEIYGI | SLLERGLEA |
| WLSSRVTQL | GVFPVVIQA | VLSSLNVPL |
| AILPKANTV | ALVHLPSQL | FLERKSIRV |
| VLLFEVELV | GLHPAKPQV | FVGSSTFYL |
| GLDTGLQGM | MLVTWELAL | FLYTGDFFL |
| SLLDGTQLF | VLLTNTIWL | SVGPFALTV |
| GLPTGYLFV | ALVHLPSQL | NLALPLPKV |
| LLDRFGYHV | CLIDEMPEA | VLESGLYQV |
| SLLEETQAI | ALMGGFMKT | MLVAITVLI |
| MLLVQPAEL | LLLHLPLXL | FMDDAKILF |
| GLLNISHTA | TLQDKNLGL | ALVHLPSQL |
| HLYEPWFPV | ILANLPPAL | ILTASITSI |
| YLQGLPLPL | PLWDGMAGL | AMDGKELSL |
| KAVEGILAV | GLDHQTHPL | SLGWNISGV |
| MIYEENNRL | GMFLLPPQL | MISAFPNEV |
| YLPYFLKSL | RLADHLSFC | RLTHELPGI |
| GLYSLSSVV | RMRDQLPAL | LLFSDGEKV |
| LMISRTPEV | GLLHAEVAL | RLNESTTFV |
| LLGGPSVFL | SLQNCQVSV | KLEELKSFV |
| ILSGYGPCV | VISAFPSEV | SINEEIQTV |
| FLPDLDRPL | ALAIAALEL | RLHDGPLRA |
| AMDGKELSL | VLDGLDVLL | MISAFPNEV |
| RMDFEDLGL | ELFPPLFMA | ILHTSVPFL |
| TLIFNPTEI | FLIVAEILI | YLENMVSGV |
| LLPGLLLLL | IVAEILISL | QLLGRLESL |
| LLLVHQHAV | KAVEGILAV | RLLHLESFL |
| FLDDAPPGT | YLPHLPQVL | ALLRQMEGI |
| VLIRYVWTL | MLLDPMGGI | TLNKDFQEV |
| YLCGHLHTL | RLLHLESFL | IMEQGDLSV |
| VLSQLTILI | YLAYILYFV | RLLHLESFL |

### 2.2.2 Validation on HLA-A2 Associated Peptides

Given that HLA-A2 allele is expressed in almost 50% of the Caucasian population, together with the existence of different technical tools, validations were focused on HLA-A2-associated peptides.

In the following paragraphs TE-Exon derived-transcripts is used interchangeably with “fusion transcripts” and the term “TE-derived peptides” is used interchangeably with “fusion transcripts-derived peptides.

### Expression of TE-Exon Derived-Transcripts in Lung Adenocarcinoma Samples

To experimentally validate the predicted TE-Exon transcripts, the expression by PCR in LUAD tumor samples and tumor cell lines was validated firstly. Specific primers for each chimeric fusion were thus designed, in order to have one of them binding to the TE part and the other to the Exon part of the fusion. The results were further confirmed by sequencing of the PCR products.

In particular, specific primers were designed in such a way that the forward primer was binding in the “donor” sequence and the reverse primer was binding in the “acceptor” sequence of the reconstructed fusion sequence. PCR reactions were run on RNA derived from lung tumor samples and human tumor cell lines. Amplifications products were seeded on agarose gels and bands found on the expected size were cut and sequenced. Finally, sequenced PCR products were compared with the reconstructed fusion sequence.

Using this approach, it was possible to confirm the presence of predicted fusion transcripts both in LUAD tumor samples and tumor cell lines. Table 4 below summarizes the results found for 8 of the most frequent chimeric fusions with a predicted peptide associated to bind with high affinity to HLA-A2 allele.

TABLE 4
Most frequent fusion transcript validation. The most frequent fusions peptides were validated by PCR in 15 LUAD tumor
samples and 6 LUAD tumor cell lines. The status ‘Yes’ or ‘No’ in the table below indicates the presence
or absence of the PCR product on the expected size. When the PCR product was further validated by sequencing, is denoted as ‘Yes’.

| | Frequency | TE-Exon fusion derived-peptides asociated to bind HLA-A2 | | | | | | | |
|---|---|---|---|---|---|---|---|---|---|
| | peptide | 119 | 48 | 28 | 24 | 23 | 19 | 18 | 16 |
| | sequence | RLLHLESFL | MLMKTVW [symbol] A | FLGTRVTRV | AILPKANTV | YLPYFLKSL | AMDGKELSL | FLIVAEILI | RLADHLSFC |
| [symbol] | [symbol] | Yes | Yes | No | No | Yes | No | No | No |
| [symbol] | [symbol] | Yes | No | No | No | No | No | No | Yes |
| [symbol] | [symbol] | Yes | No | No | No | No | No | No | Yes |
| | [symbol] | Yes | Yes | No | No | Yes | No | No | No |
| | [symbol] | Yes | No | No | No | No | No | No | No |
| | [symbol] | Yes | Yes | No | No | Yes | [symbol] | No | No |
| [symbol] | [symbol] | Yes | No | No | No | Yes | Yes | No | Yes |
| [symbol] | [symbol] | Yes | Yes | No | No | Yes | Yes | No | No |
| [symbol] | [symbol] | Yes | No | No | No | Yes | No | No | Yes |
| | [symbol] | Yes | No | No | No | Yes | Yes | No | No |
| | [symbol] | Yes | No | No | No | No | No | No | No |
| | [symbol] | Yes | No | No | Yes | Yes | Yes | Yes | Yes |
| | [symbol] | Yes | No | No | No | No | Yes | No | No |
| | [symbol] | Yes | No | No | No | Yes | Yes | No | No |
| | [symbol] | Yes | No | No | No | No | Yes | Yes | No |
| | [symbol] | Yes | Yes | Yes | Yes | Yes | Yes | Yes | No |
| | [symbol] | Yes | Yes | No | No | Yes | Yes | Yes | No |
| | [symbol] | Yes | Yes | Yes | Yes | Yes | Yes | No | No |
| | [symbol] | Yes | Yes | No | Yes | Yes | No | No | Yes |
| | [symbol] | Yes | Yes | Yes | Yes | Yes | Yes | No | Yes |
| | [symbol] | No | No | No | No | No | Yes | No | No |

[symbol]

indicates data missing or illegible when filed

### Binding of ER-Derived Peptides to HLA-A2 Molecule

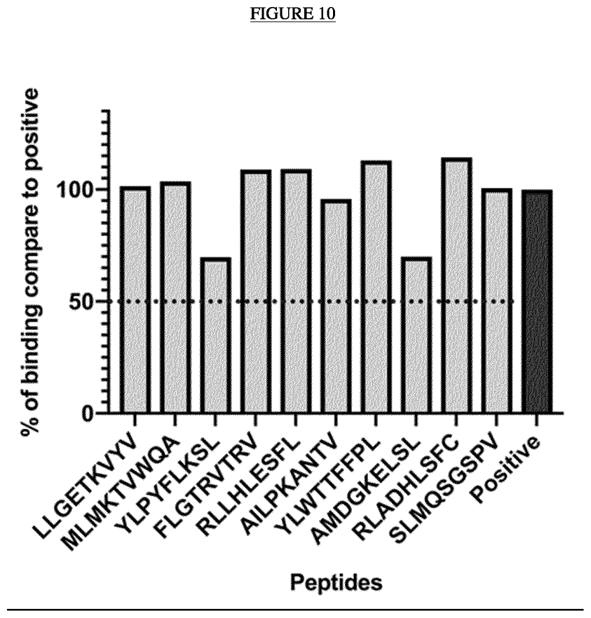

Once confirmed the expression of chimeric transcripts, the derived-peptides were synthetized and their binding to HLA-A2 was confirmed. Because monomer stabilization and tetramer formation are only possible in the presence of a high affinity binding peptide, the formation of HLA-A2 tetramers was estimated in the presence of synthetized peptides by flow cytometry. All predicted peptides were able to stabilize tetramer formation, showing a percentage of fluorescence higher than 50% relative to positive control. As positive control, a known high affinity binding peptide to HLA-A2 derived from Cytomegalovirus (CMV) was used. This result confirmed the predicted high affinity binding to HLA-A2 allele. FIG. 10 shows the result for 10 peptides derived from the most frequent fusions peptides.

FIG. 17 shows a new set of peptides, also derived from frequent chimeric transcripts, with a confirmed binding to HLA-A2 using the same peptide-MHC-I complex formation assay. As a positive control of complex formation, we used both CMV pp65 495-503 (NLVPMVATV) and the mutated sequence of Melan-A (MelA mut, ELAGIGILTV), both known good binders to HLA-A2. The non-mutated sequence of Melan-A (MelA) was used as a control of low binder peptide. Negative is recombinant HLA-A2 molecule without any peptide.

### Immunogenicity of ER-Derived Peptides

The following step after binding validation to HLA-A2 allele, was to test the immunogenicity of predicted peptides. Priming assays were thus performed to test the ability of identified peptides to expand specific cytotoxic T cells. PBMCs from HLA-A2+ healthy donors were used to generate monocyte derived-DCs (moDCs). After loading the moDCs with a mix of synthetic peptides, autologous co-culture was performed with CD4+ and CD8+ T cells. Finally, the expansion of specific CD8+ T cells was analysed by flow cytometry using two-colours tetramer staining. As a control of specific expansion, the co-culture was performed in the absence of peptides. By using this approach in one donor, it has been possible to identify and expand specific CD8+ T cells recognizing 6 of the most frequent chimeric fusion derived-peptides (RLLHLESFL, LLGETKVYV, AILPKANTV, RLADHLSFC, FLIVAEILI, YLWTTFFPL). This result is evidenced by an increase in at least one magnitude order of the percentage of tetramer positive cells compared to control test among total CD8+ T cells.

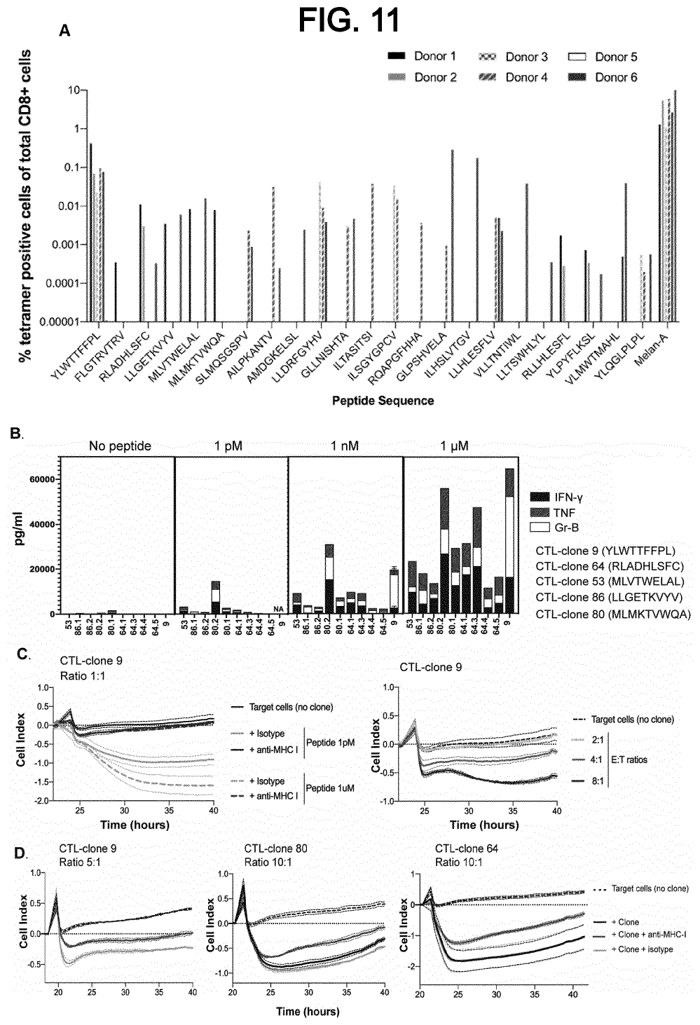

The same experiment was performed in order to evaluate the response in additional 5 donors. FIG. 11A summarizes the results obtained for the total of 6 donors analyzed in which we found specific CD8+ T expansions for 23 out of 29 of the most frequent fusions transcripts-derived peptides (YLWTTFFPL, FLGTRVTRV, RLADHLSFC, LLGETKVYV, MLVTWELAL, MLMKTVWQA, SLMQSGSPV, AILPKANTV, AMDGKELSL, LLDRFGYHV, GLLNISHTA, ILTASITSI, ILSGYGPCV, RQAPGFHHA, GLPSHVELA, ILHSLVTGV, LLHLESFLV, VLLTNTIWL, LLTSWHLYL, RLLHLESFL, YLPYFLKSL, VLMWTMAHL, YLQGLPLPL). As a positive of expansions, mutated Melan-A peptide (ELAGIGILTV) were used. These experiments show that these peptides are able to induce an immune response and confirms the immunogenicity of ER-derived peptides.

### Generation of Cytotoxic T Lymphocytes Clones Recognizing ER-Derived Peptides

Expanded CD8+ tetramer positive T-cells from immunogenicity assays (FIG. 11A) were single cell FACS-sorted in order to generate cytotoxic T lymphocytes (CTLs) clones. 10 clones recognizing 5 different ER-derived peptides were generated: YLWTTFFPL, LLGETKVYV, MLVTWELAL, MLMKTVWQA, RLADHLSF. These peptides are listed in Table 3 as peptide 9, 86, 53, 80 and 64 respectively. It will be referred to these numbers to indicate the specificity of each generated CTL-clone. For example, CTL-clone 9 recognize ER-derived peptide 9. In a second set of experiments a new CTL-clone 17 was generated recognizing peptide 17 (LLDRFGYHV).

In order to evaluate the cytotoxic capacity of generated CTL-clones, two different functional assays were conducted using the H1650 cell line as target cells. This is a LUAD-derived tumor cell line expressing HLA-A2 allele.

First, the ability of CTL-clones to secret cytokines after exposure to ER-derived peptides was measured. After co-cultured of the CTL-clones with the target cells loaded with the specific ER-derived peptides during 18h, secretion of INF-y, TNF and Granzyme-B (Gr-B) was measured in culture supernatants. All CTL-clones were activated after exposure specific ER-derived peptides, secreting cytokines in a dose-dependent manner (FIG. 11B).

In a second set of experiments, CTL clones killing capacity was assessed. CTL-clones were co-cultured in different conditions with target cells loaded or not with ER-derived peptides. Using xCELLigence system the real-time impedance variation in a target cells monolayer was measured. In these assays, a decrease in cell-index is related with a decrease in the number of cells in the monolayer reflecting cell viability.

When CTL-clone 9 was co-culture in 1:1 ratio with target cells loaded with ER-derived peptide 9, a decrease in cell-index over time was observed, compared to the control cells (target cells alone). This cell-index decrease was inhibited when co-culture was performed in the presence of blocking anti-MHC-I antibody (+ anti-MHC-I). Performing the co-culture using the same concentration of isotype control (+ isotype) did not inhibit the decrease in cell-index. Moreover, the amount of the decrease increased when target cells were loaded with a higher concentration of peptide (1 pM compared to 1 uM) (FIG. 11C, left panel). These result show that cytotoxic T cells can recognize peptides encoded by a fusion transcript as herein described and kill target tumor cells expressing such peptides.

It was then demonstrated that ER-derived peptides are naturally expressed and presented by target cells, such said target target-cells can thus be killed by co-culturing them with CTL-clones without external addition of peptides. To this aim, co-culture of CTL-clone 9 with H1650 target cells at different ratios were performed. The right panel of FIG. 11C, shows that CTL-9 was able to kill target cells at a ratio effector-target of 4:1 as compared to the control cells (target cells alone). Moreover, killing efficacy is increased at higher ratios (8:1). No killing of target cells was evidenced at lower ratios (2:1).

Finally, similar experiments were performed with CTL-clone 9, CTL-clone 64, and CTL-clone 80 showing a specific killing of target cells that could be also inhibited when the co-culture is performed in the presence of anti-MCH-I antibodies (FIG. 11D).

All together, these results confirm that cytotoxic T cells that recognizes several different peptides encoded by a fusion transcript as herein described can recognize and kill tumor cells expressing said specific fusion transcripts-derived peptides and that this effect is due to the specific recognition of peptides in the context of MHC-I molecules. Moreover, the fact that CTL-clones are able to kill target cells without addition of external peptides, provide clear evidence that fusion transcripts-derived peptides are naturally expressed and presented by an LUAD tumor cell line.

### Generation of Engineered T-Cells Recognizing Fusion-Derived Peptides

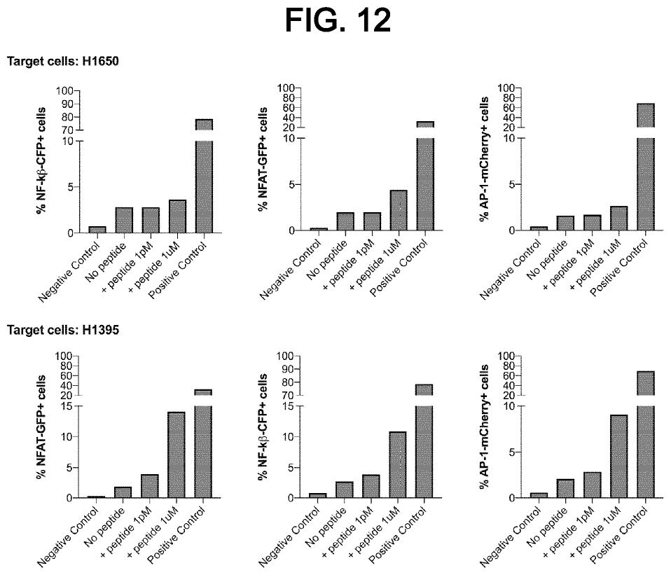

Jurkat cells transduced with lentiviral vector encoding for CTL-9 TCR sequence were co-cultured with two different target cells, H1650 and H1395. Both are LUAD-derived cell lines express HLA-A2 allele. TCR-mediated activation of Jurkat cells was evaluated by flow cytometry as an increase in the fluorescence of reporter genes (NFAT-GPF, NF-KB-CFP and AP-1-mCherry). Preliminary results showed that Jurkat cells are activated when co-cultured with both target cells compared to negative control (non-transduced Jurkat cells). Furthermore, this activation increased in a dose-dependent manner when the co-culture was performed with target cells loaded with specific peptides. PMA/ionomycin was used as positive control (FIG. 12).

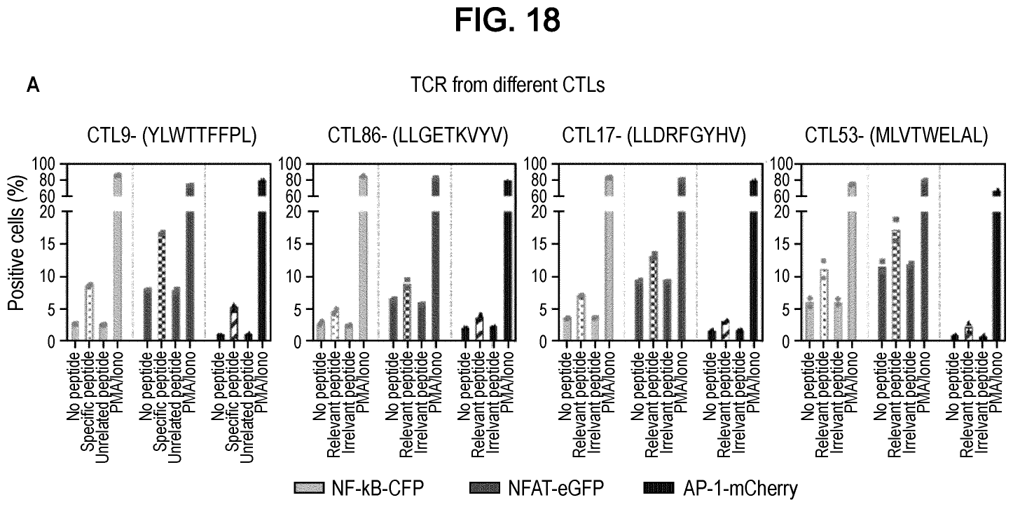

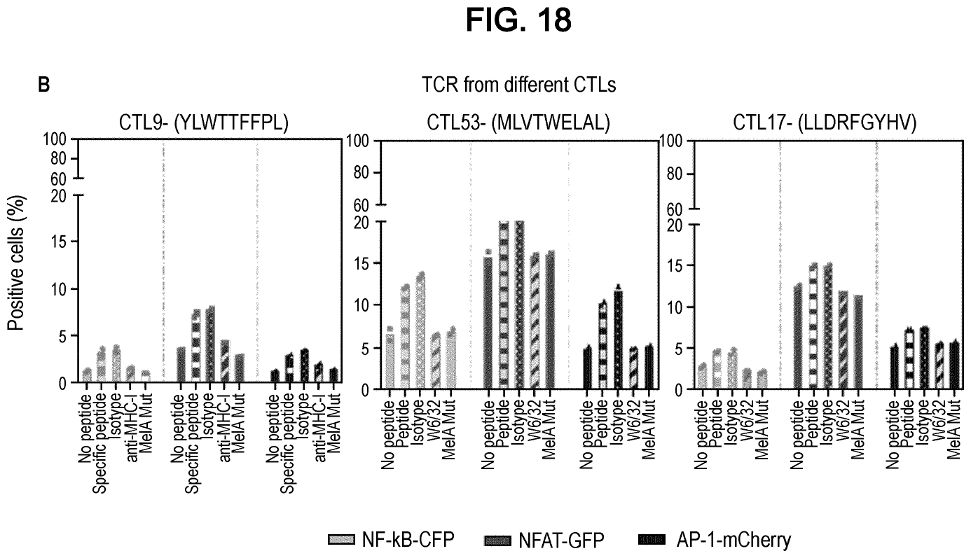

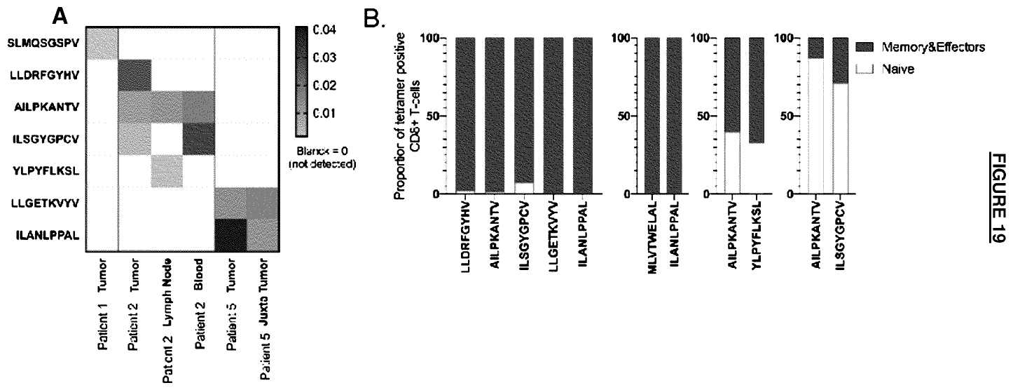

These results were repeated in another set of experiments and similar ones were obtained with Jurkat cells transduced with lentiviral vector encoding TCR sequences from CTL-86 and CTL-53 and CTL-17. Transduced Jurkat cells were activated by co-culture with a target tumor cell line loaded with the corresponding ER-derived peptide (Specific/Relevant peptide) but not with the control Melan-A peptide (Unrelated/Irrelevant peptide, ELAGIGILTV) (FIG. 18A). As expected, activation is inhibited by blocking with anti-HLA-I antibodies (FIG. 18B). TCRs expressed by the generated CTL-clones are thus specific to the corresponding HLA-A2 presented ER-derived peptides. Table 4bis lists the sequences of re-expressed TCRs.

These results are in line with the results shown in FIGS. 11 C and D, showing that LUAD-derived tumor cells express TE-derived peptides. Furthermore, these results also highlight the technical relevance of CTL-clones TCR sequences in the development of engineered T cells.

TABLE 4bis
TCR sequences derived from CTL-clones recognizing chimeric transcripts-derived peptides.

| TCR | | | | | | | | |
|---|---|---|---|---|---|---|---|---|
| num- | Speci- | TCRalpha | | TCRbeta | | | | |
| ber | ficity | V | J | V | D | J | CDR3alpha | CDR3beta |
| 1 | LLDRFGYHV | TRAV41*01 | TRAJ34*01 | TRBV6-5*01 | TRBD1*01 | TRBJ- | CAVLLDNTDKLIF | CASSRLQGALELFF |
| | | | | | | 2*01 | | |
| 2 | YLWTTFFPL | TRAV12*2*03 | TRAJ44*01 | TRBV7-6*01 | TRBD1*01 | TRBJ2- | CAVNTRGTASKLTF | CASSLGGGTDTQYF |
| | | | | | | 3*01 | | |
| 3 | LLGETKVYV | TRAV12-3*01 | TRAJ49*01 | TRBV2*02 | TRBD2*01 | TRBJ- | CAVFTGNQFYF | CASLSGESYEQYF |
| | | | | | | 7*01 | | |
| 4 | MLVTWELAL | TRAV35*01 | TRAJ54*01 | TRBV13*01 | TRBD2*01 | TRBJ2- | CAGQLAGAQKLVF | CASSPSRDGQETQYF |
| | | | | | | 5*01 | | |

### Presence of CD8+ Cells Recognizing Fusion-Derived Peptides in LUAD Patients

It was then aimed to identify the presence of CTL cells recognizing fusion-derived peptides in LUAD tumor samples.

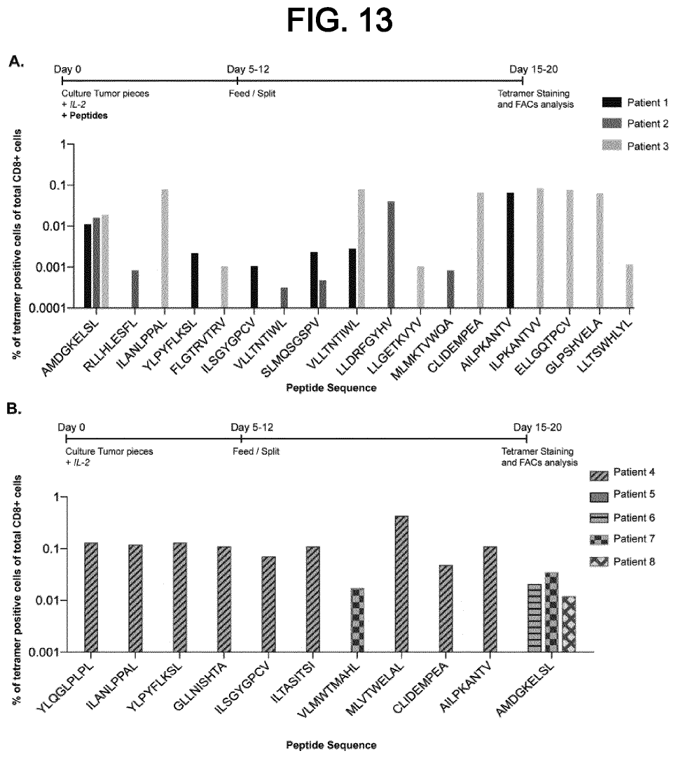

In a first set of experiments tumor infiltrating lymphocytes (TILs) expanded with a mix of TE-derived peptides and Il-2, or only with Il-2, were analyzed by tetramer staining. As is shown in FIGS. 13A and B, CD8+ T-cells cells recognizing fusion-derived peptides were found in TILs derived from LUAD patients.

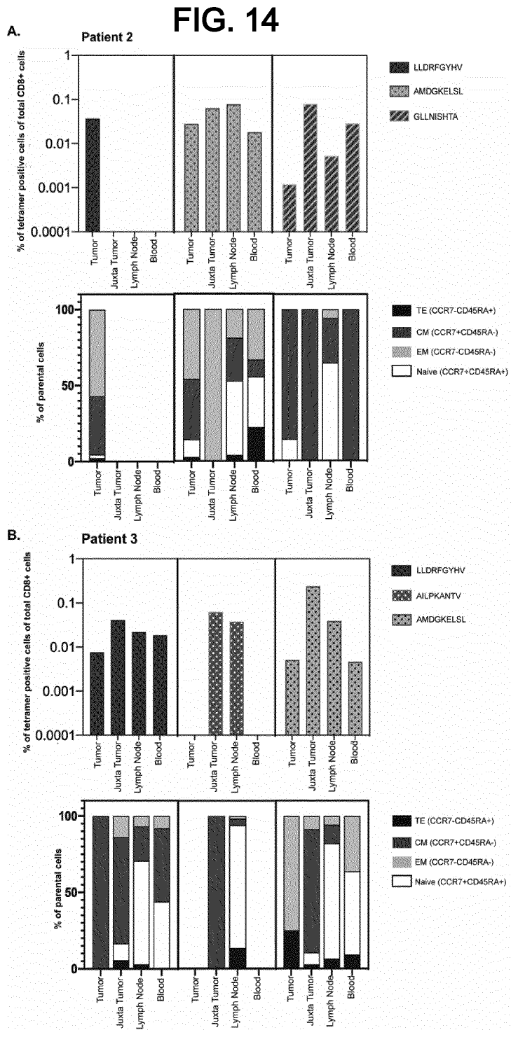

It was then showed that tetramer positive cells could be detected and their phenotype in non-expanded CD8+ T cells derived from fresh tumor samples was further assessed. Using this strategy, CD8+ T cells present in Tumor, juxta-tumor, invaded lymph-nodes and blood derived from LUAD patient samples were thus analyzed. The cell phenotype was determined based on the expression of surface markers CCR7 and CD45RA for Naïve (CCR7+CD45+), Central Memory (CM, CCR7+CD45RA−) Effector Memory (EM, CCR7−CD45−) and Terminal Effectors (TE, CCR7−CD45+) T cells. Interestingly, tetramer positive cells found in tumor tissues shared preferentially a memory phenotype whereas naïve cells (CCR7+CD45+) are found mostly in cells derived from lymph nodes (FIGS. 14A and B). Patient 2 and 3 are the same in FIG. 13 and FIG. 14.

All samples tested derived from HLA-A2+ patients.

The presence of tetramer positive cells with a memory phenotype in tumor tissues, together with the presence of tetramer positive cells in TILs, provide evidence that an immune response is generated against TE-derived peptides in these patients. Moreover, the existence of naïve tetramer positive cells in lymph nodes shows that an immune response against these particularly TE-derived peptides can be generated.

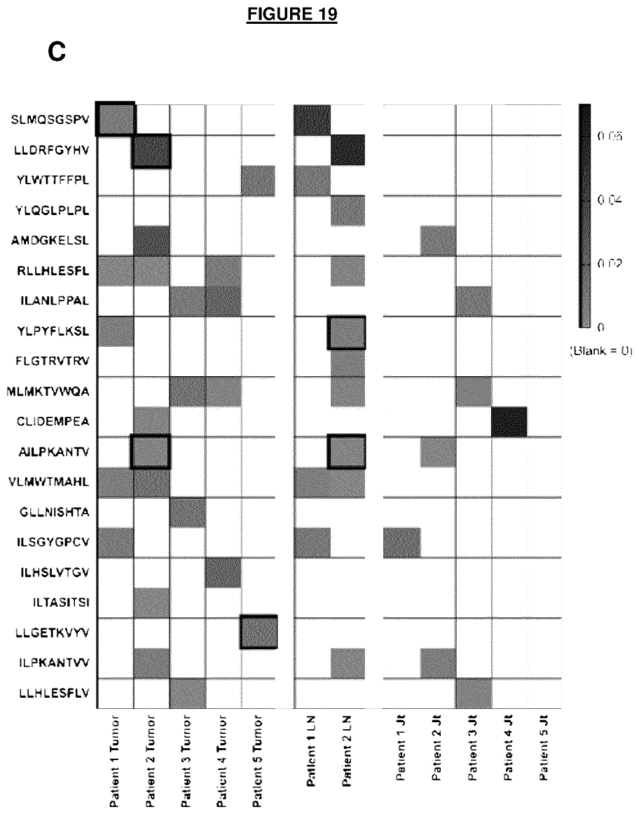

In a second cohort of 5 primary, untreated, LUAD tumor, juxta-tumor, tumor-draining lymph node and blood samples from LUAD cancer HLA-A2+ patients were analyzed. Half of each sample was analyzed directly ex-vivo by isolating CD8+ T cells without in-vitro expansions, and the other half was cultured in-vitro for 20 days either with chimeric transcript-derived peptide mixed with IL-2 (patients 1 and 2) or with IL-2 alone (patients 3, 4, 5), aiming to amplify in the samples, the populations of specific T cells recognizing Chimeric transcript-derived peptides. T cells were identified using double color tetramer staining. Antibodies directed CCR7 and CD45RA were also added to the non-expanded cells to distinguish naïve and memory cells. Expansions were considered with 5 or more double tetramer-labelled cells. FIG. 19A shows a summary of the 7 “Chimeric transcript-derived peptide specific” tetramer-positive T cell populations found in the 4 patients analyzed directly ex-vivo (one of the patient samples could not be analyzed for technical reasons). CCR7/CD45RA labeling showed that all tetramer-positive T cells detected in tumor samples have a clear effector/memory phenotype, whereas in blood and lymph nodes the “Chimeric transcript-derived peptide specific” tetramer-positive T cells have variable proportions of less differentiated, CCR7+naïve and/or central memory phenotypes.

Therefore, these results demonstrate that primary human NSCLC tumors contain chimeric transcript-derived peptide specific memory T cells (FIG. 19B).

A summary of expanded, tetramer+CD8+ T cells, is shown in FIG. 19C. For the majority of peptide specificities, T cells were expanded from both the tumor and the matched invaded lymph nodes (LN) analyzed only in 2 patients, and in some cases from the matched juxta-tumor samples (Jt) (FIG. 19C). 5 out of 7 specific tetramer positive populations were also found at Day 20 in the same patient and tissue found ex-vivo without T cell expansions (FIG. 19A and bold squares on FIG. 19C).

These results provide thus evidence that chimeric transcript-derived peptide specific T cells are present in tumors, tumor-draining lymph nodes and sometimes in juxta-tumor tissue and blood of LUAD patients before and after in-vitro expansion, consistent with the existence of chimeric transcripts-derived peptide specific immune responses in LUAD patients.

### Peptide Identification by Mass Spectrometry in LUAD Biopsies.

Presentation by MHC class I molecules on the tumour cell surface is required for ER-derived peptides in order to be recognized by cytotoxic T cells. In order to confirm that predicted peptides are express on MHC class I molecules, public data from MHC I immunopeptidome derived from 3 LUAD biopsies (Laumont C M et al., “Noncoding regions are the main source of targetable tumor-specific antigens” Sci Transl Med. 2018 10 (470)) were used. OpenMS Software was used to analyse the raw data uploaded to PRIDE database from MHC-I immunopurification of 3 LUAD tumours (PXD009752, PXD009754 and PXD009755). Having in mind that data-dependent acquisition in proteomics only allows the identification of those sequences contained in a target database (generally the whole human proteome); the peptides as per the present application had not been previously identified because they derive from non-coding sequences. The MS/MS identifications incorporating the sequences of the herein predicted peptides in the target database has been re-analyzed. Five peptides among the 3 samples biopsies (peptides ID: 3304, 269, 757, 1810, 3953) were found. To perform this analysis, all predicted peptides derived from chimeric fusions present in at least 5 samples in the TCGA binding to any MHC I allele were considered. This result confirms the expression of chimeric fusion-derived peptides on MHC class I molecules in LUAD tumors.

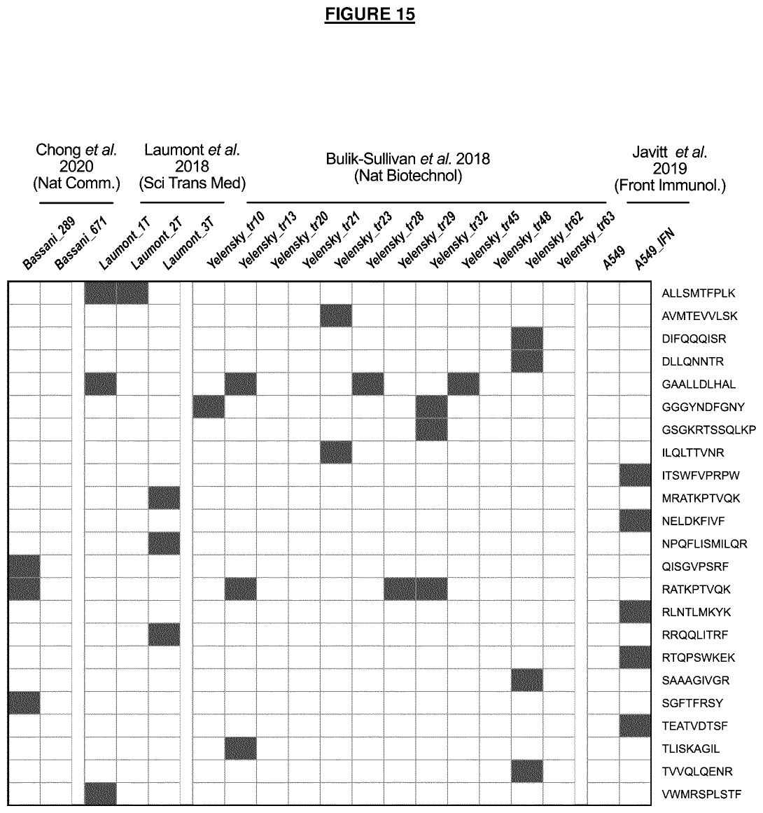

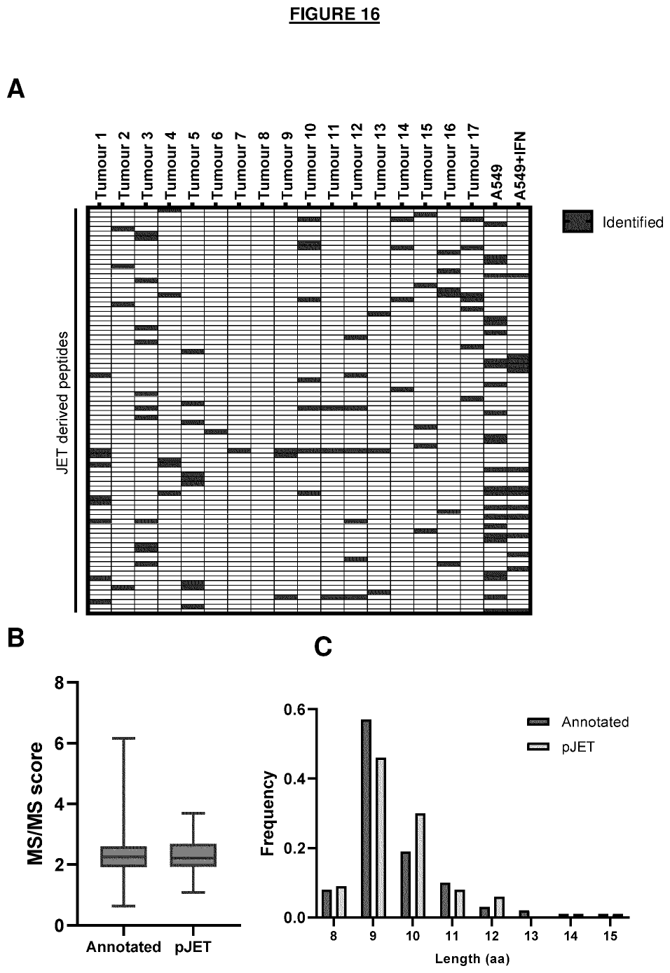

Later, we extended our analysis to new lung immunopeptidomics datasets (Bulik-Sullivan et al. Nat. Biotec 2018, Chong et al. Nat. Comm. 2020 and Javitt et al. Front Immunol 2019). Of note, all datasets were generated with fresh lung tumor samples with the exception of Javitt et al. Front Immunol 2019 containing LUAD tumor cell line. For this second analysis, ProteomeDiscoverer 1.4 Software was used to identify the ER-derived peptides. Considering the 4 datasets, 23 unique ER-derived peptides were present in at least one of the total 19 immunopeptidomic samples. In FIG. 15, ER-derived peptides (rows) identified in each MHC sample (column) are indicated with a grey square. On the right, the peptide sequence found is indicated. Interestingly, some of them were observed in more than 1 MHC sample indicating that they are shared across samples. These results confirm that fusion transcripts-derived peptides are processed and presented by HLA-I molecules on tumor cells surface.

Peptide RLADHLSFC derived from a fusion transcript where the gene part of the fusion is a tumor suppressor gene (Fusion ID: chr22: 29117506:->chr22: 29115473:-/gene involved: CHEK2) and peptide GLPSHVELA derived from a fusion transcript where the gene part is an oncogene (Fusion ID: chr6: 117763597:->chr6: 117739669:-/gene involved: ROS1). Interestingly, both peptides were found to be immunogenic (FIG. 11A) and particularly for peptide RLADHLSFC, results show in FIG. 11 D indicate that could be express by H1650 cell line. Furthermore, we found TILs recognizing peptide GLPSHVELA (FIG. 12A), which indicates that this fusion transcript-derived peptide could be express in LUAD tumor samples.

### Example 3: Identification Neoantigenic Peptides Derived from Fusion Transcripts Composed of a TE Element and an Exonic Sequence from Various Cancer Samples

9184 samples from 32 different cancer types (Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma and Uveal Melanoma) were analyzed according to the method as previously described.

16580 fusion transcripts were identified.

### 4 Example 4: Proteomics Results

### Results

### Total Proteomics

Mass spectrometry-based proteomics has emerged as a powerful tool to interrogate the actual protein content of a given cell preparation. To confirm that JETs are indeed translated into proteins, mass spectrometry output files (called raw files) generated from cell lines and fresh tumors were analyzed to identify different populations of JET-derived peptides. This study has been grouped into two different analyses, each one providing a different and complementary type of information, that demonstrate that JET derived proteins can reliably be detected in a tumor sample or in a tumor cell line.

First it was demonstrated that proteins derived from the JETs were found to be highly recurrent in CCLE dataset. Therefore, the in-silico translated junctions from all those JET mRNA sequences predicted in more than 7 different cell lines in the CCLE cohort were used and interrogated to the mass spectrometry raw files from Nusinow et al. 2020, which consists in the proteomics analysis of 375 cell lines from CCLE. These cell lines were grouped in TMT10plex, generating a total amount of 42 MS/MS output files. This MS-based proteomics analysis led to the identification of 186 JET derived proteins, containing at least 1 peptide overlapping the splicing junction (Table 5).

TABLE 5

| | | Recurrence |
|---|---|---|
| Sequence | Chimeric_id | in dataset |
| SSTGPADLK | chr20:42212014:+ > chr20:42213492:+ | 72 |
| MLLGLLAK | chr7:20686997:+ > chr7:20687158:+ | 72 |
| MGISIVK | chr8:74872000:− > chr8:74871067:− | 72 |
| IQSFTNISFSMPHR | chr1:44680991:+ > chr1:44683983:+ | 63 |
| TQEVEVAVNR | chr7:73776406:+ > chr7:73778585:+ | 45 |
| MASASPTIK | chr3:53915693:− > chr3:53914136:− | 36 |
| ETDLITVTGSSFLTHR | chrX:57620887:+ > chrX:57667223:+ | 36 |
| EAENEVQTAK | chr7:116555100:+ > chr7:116556114:+ | 36 |
| LNIHLLMVTSSVTSSSILINLDR | chr13:41835827:− > chr13:41835011:− | 36 |
| DLELEIPFGPAIPLLGSCSQCR | chr11:129978600:+ > chr11:129979324:+ | 36 |
| MILESTYHNTLHIAMK | chr6:17835789:− > chr6:17834302:− | 36 |
| MLAPENSDIGLAAWGR | chr20:32888526:− > chr20:32883391:− | 36 |
| TQEAELAGVATVR | chr3:122863676:+ > chr3:122864369:+ | 27 |
| TSLHSIPVTVGRR | chr17:42188097:− > chr17:42182463:− | 27 |
| AEIAPLHSSLGDK | chr5:132253438:− > chr5:132240096:− | 27 |
| MLGAVAHNCDPSTLR | chr12:57081782:− > chr12:57080459:− | 27 |
| GPAPEPPSMR | chr19:50010181:+ > chr19:50027764:+ | 27 |
| MACNMGLEKGEK | chr5:179238682:+ > chr5:179250858:+ | 27 |
| MASICPFLK | chr20:49307663:− > chr20:49307455:− | 27 |
| EIETILANPENK | chr6:130374597:+ > chr6:130376316:+ | 27 |
| QAQCSTDSTLWYFMHGVCR | chr3:12604393:+ > chr3:12610374:+ | 18 |
| LASTTSVSEEDVSSR | chr12:79996543:− > chr12:79990438:− | 18 |
| AIAQGIQELR | chr17:43475315:− > chr17:43474814:− | 18 |
| TCPSAMIVSFLK | chr10:51747031:+ > chr10:51749068:+ | 18 |
| VQLAGNTLPSTPCWTR | chr9:130924301:+ > chr9:130925722:+ | 18 |
| GLEFETSLANVVQLPNSIVFSVTR | chr18:19147325:− > chr18:19146167:− | 18 |
| KESLEASMMLK | chr9:88264917:− > chr9:88261333:− | 18 |
| VETLCATLWGFGK | chr10:102711867:+ > chr10:102716208:+ | 18 |
| MMAHLCIFGLQSR | chr5:86704905:− > chr5:86704003:− | 18 |
| VSTSHVGPWVSP | chr5:1475076:− > chr5:1474800:− | 18 |
| NLLDIDAPVTGWQELK | chr4:102117073:− > chr4:102104428:− | 18 |
| AKIAPAEGPDVSER | chr3:185370866:− > chr3:185369956:− | 18 |
| EAEEPSDNGPLFTELK | chr14:97307963:+ > chr14:97312432:+ | 18 |
| MAGLVFQWLR | chr3:136005373:+ > chr3:136012598:+ | 18 |
| ISQLQIWDTAGQER | chr3:128828867:− > chr3:128814012:− | 18 |
| VISAFPSELQSQCTK | chr6:100379769:− > chr6:100369131:− | 18 |
| TAQLPEQLMTWHSR | chr22:37870550:− > chr22:37861756:− | 18 |
| LLNITAGLGSDGTIR | chr6:34511797:− > chr6:34511385:− | 18 |
| MGTVAHAYNPSTLGGR | chr2:48593264:+ > chr2:48600431:+ | 18 |
| MLPPFVLAVTMGLSVVK | chr3:15058779:+ > chr3:15062260:+ | 18 |
| QADHLMPGVQDQPNMTSVP | chr11:18491675:− > chr11:18490765:− | 18 |
| WAGHSASCSSLRK | chr22:18351213:− > chr22:18348778:− | 18 |
| TFMMIHCHLMNNMSCR | chr3:10160654:+ > chr3:10167310:+ | 9 |
| GQIGILLWNSMGTMQQPR | chr20:30254794:− > chr20:30253889:− | 9 |
| APTDSTLCTLCK | chr17:62609987:− > chr17:62602758:− | 9 |
| LREPIDNLTPEER | chr20:34315958:− > chr20:34313077:− | 9 |
| EWSGIQKATLMDLQLSDSNFRR | chr18:244302:− > chr18:226901:− | 9 |
| TSLANMVVFPLMK | chr2:98263129:+ > chr2:98263530:+ | 9 |
| IWLTEDATALLQRPGWR | chr11:44960717:− > chr11:44959917:− | 9 |
| METGIISLLPPWGGRPSAVR | chr1:6531548:− > chr1:6531300:− | 9 |
| GNLLIISLDAK | chr17:65894534:+ > chr17:65899905:+ | 9 |
| TSSGLPLILHCVYAK | chr16:82201145:− > chr16:82197799:− | 9 |
| SGVQDQPGQQGPVTLHK | chr10:28824988:+ > chr10:28872328:+ | 9 |
| WADHLTSGVR | chr10:95225471:− > chr10:95216694:− | 9 |
| MNDSCIFRPETDSDIMELTDR | chr19:19976808:+ > chr19:19982937:+ | 9 |
| IVEQTLK | chr13:115035095:+ > chr13:115037659:+ | 9 |
| MELTIPGEGK | chr2:99224660:− > chr2:99220654:− | 9 |
| HFIAMMYFK | chr5:145540727:− > chr5:145540049:− | 9 |
| ETFFLQQQGNDAK | chr16:89607754:+ > chr16:89611056:+ | 9 |
| QLQVCVLQIMGSCVPSCPTP | chr11:64643083:− > chr11:64641990:− | 9 |
| EAEASQAAQEAEINAR | chr1:183107957:+ > chr1:183109539:+ | 9 |
| YSNVLEMAWLQYA | chr12:63961380:− > chr12:63954442:− | 9 |
| MATVAHTSLATVEDFQIRPHTLY | chr2:37520065:− > chr2:37518142:− | 9 |
| VHSYK | | |
| LPIFFFGTHETLLGGR | chr9:15506559:− > chr9:15492223:− | 9 |
| QASRALPPCPWNTQTSGSR | chr10:6010739:− > chr10:6008302:− | 9 |
| LDIGFPSLQNYR | chr17:47441711:+ > chr17:47450375:+ | 9 |
| TLPSLDDLR | chr7:72720556:− > chr7:72719094:− | 9 |
| LLVNDPETAVK | chr12:112149997:+ > chr12:112150302:+ | 9 |
| LQEMGPLGVPQR | chr14:105335983:+ > chr14:105342593:+ | 9 |
| TSLANMDMFK | chr12:72301769:+ > chr12:72307606:+ | 9 |
| MGAVVHACKPSTLGGR | chr15:42151606:− > chr15:42151178:− | 9 |
| KQAQKDCPTHSGVS | chr17:43536749:− > chr17:43531638:− | 9 |
| SEGIRIMEMLLK | chr5:145153908:− > chr5:145144563:− | 9 |
| GFGPSTMLGTQK | chr1:193028315:− > chr1:193022970:− | 9 |
| MTKLLSPIR | chr22:32787416:− > chr22:32784086:− | 9 |
| WLTPVIPALWEAER | chr8:103282996:− > chr8:103282411:− | 9 |
| ETTLAFK | chr1:240569786:+ > chr1:240601361:+ | 9 |
| ATALQPGR | chr1:45988450:− > chr1:45981479:− | 9 |
| ITSVVMVDEAHER | chr6:30629093:− > chr6:30628019:− | 9 |
| SQYCTPALEAETK | chr17:78263365:+ > chr17:78263458:+ | 9 |
| TLTFLGPLQFR | chr19:20025631:+ > chr19:20026089:+ | 9 |
| TPEVEATVPLQK | chr22:42085308:+ > chr22:42089467:+ | 9 |
| VILIFFPECYAK | chr22:31947110:+ > chr22:31952928:+ | 9 |
| DGDHPSQHDGVLLCCPGWSTM | chr9:96211969:− > chr9:96210771:− | 9 |
| GGSHLQSQHFGRPR | chr21:34973474:− > chr21:34971554:− | 9 |
| SVSTIFLTCSR | chr10:1100108:− > chr10:1090111:− | 9 |
| MGTVAHACNPCTLGG | chr20:17550856:+ > chr20:17565018:+ | 9 |
| TPGRNGHLESGHLK | chr7:102793520:+ > chr7:102939015:+ | 9 |
| SGFSFLLGIVGF | chr12:22831248:+ > chr12:22837417:+ | 9 |
| EIETLHHPSTGVILADHK | chr13:44432917:− > chr13:44413224:− | 9 |
| TIAQMIDSTVALPLEAT | chr8:38697785:+ > chr8:38698832:+ | 9 |
| NQSQSHLNVGLLASR | chr9:21808999:+ > chr9:21815432:+ | 9 |
| SVERFLQEDQMFHQEK | chr9:97843062:+ > chr9:97909493:+ | 9 |
| MELTIPGVGHSSGSLNEGK | chr2:99224633:− > chr2:99220654:− | 9 |
| ESLEASMMLK | chr9:88264917:− > chr9:88261333:− | 9 |
| LLITAEDQSMWIQS | chr13:113897987:+ > chr13:113898724:+ | 9 |
| QAWWIMPIIPALWEAK | chr2:10135488:+ > chr2:10136007:+ | 9 |
| MESQMVEQLCSR | chr8:66633382:− > chr8:66631730:− | 9 |
| GLTGEFSITR | chr5:1257816:− > chr5:1255526:− | 9 |
| GGEFAPSWDCKP | chr2:37490123:− > chr2:37487527:− | 9 |
| CLTTLFGSFR | chr7:107256834:+ > chr7:107258773:+ | 9 |
| SQHFGRPSCVPLPATGVK | chr12:69249761:− > chr12:69236109:− | 9 |
| EQLSQAEAVALK | chr6:17837109:− > chr6:17835895:− | 9 |
| GQVLGPWPSR | chr19:1045618:+ > chr19:1046229:+ | 9 |
| TGAQPPATMPLNVSFTNR | chr8:128749923:+ > chr8:128750494:+ | 9 |
| TGIPSATLIR | chr22:46193106:+ > chr22:46202839:+ | 9 |
| EQTDVMHQNIYD | chr5:414855:+ > chr5:422844:+ | 9 |
| FVDVTECQACSANSR | chr22:25019883:+ > chr22:25023093:+ | 9 |
| IRDSEELIGR | chr15:79649214:+ > chr15:79703742:+ | 9 |
| DHIMFSLFEK | chr1:116206078:+ > chr1:116206282:+ | 9 |
| DSGSTTWCFIQK | chr12:112169999:+ > chr12:112171727:+ | 9 |
| GFPSHMSLGYHFQCPIPR | chr2:28190310:+ > chr2:28210860:+ | 9 |
| CPEMLPTSQTLR | chr12:110470515:+ > chr12:110471602:+ | 9 |
| FLQQCLLLLVSLEVFR | chr19:54502735:+ > chr19:54515205:+ | 9 |
| TAGGQQQALPLGSPR | chr17:45356073:+ > chr17:45360720:+ | 9 |
| SVTVSHNPQLGPSGCR | chr5:133691774:− > chr5:133686118:− | 9 |
| AVTANGYQMLDGK | chr2:43779972:− > chr2:43779478:− | 9 |
| FPSGILCVIMPGR | chr6:3432328:− > chr6:3416089:− | 9 |
| HDYDEDLVQEASSEDVLGVHMV | chr5:177450333:+ > chr5:177462097:+ | 9 |
| DK | | |
| CNPSTLGDQAEK | chr2:231040909:− > chr2:231037675:− | 9 |
| AEISCQQPAPGPR | chr19:49832179:+ > chr19:49838971:+ | 9 |
| GQEMETIPANTLIQEDILDTGNDK | chr12:86274547:+ > chr12:86276001:+ | 9 |
| GGFHHDCK | chr7:84736037:− > chr7:84727281:− | 9 |
| LWTSLPAPMFSR | chr20:60835987:+ > chr20:60838672:+ | 9 |
| CMCLEYPAVVEFAPFQK | chr13:115049839:+ > chr13:115051777:+ | 9 |
| LIISIFLVHSPK | chr12:7125578:− > chr12:7120720:− | 9 |
| GATWTGSSPQGSWDFTR | chr7:75083158:− > chr7:75070925:− | 9 |
| TVTVPAQELK | chr9:128421519:− > chr9:128420078:− | 9 |
| ENTMAENELQV | chr10:112572705:+ > chr10:112576399:+ | 9 |
| TPMELELPPASHFEFIGSSANCR | chr2:37570066:+ > chr2:37579932:+ | 9 |
| GLLWSGMALTGSLRVTQR | chr22:36007617:− > chr22:36007153:− | 9 |
| GLSDHVQLSQSHSTSFHVSPK | chr16:90158141:− > chr16:90141431:− | 9 |
| MILESTYHNTLHIAMKAPELK | chr6:17835789:− > chr6:17834302:− | 9 |
| CPETFLDGTLGR | chr19:4703015:− > chr19:4702728:− | 9 |
| SYQLRPGTMIEWGNYWR | chr7:56052571:+ > chr7:56059164:+ | 9 |
| TILANTVMAR | chr16:2115231:+ > chr16:2115520:+ | 9 |
| MELTIPGVGK | chr2:99224656:− > chr2:99220650:− | 9 |
| SSPQAIQMVATLGTTTCCSFDNLL | chr7:50595367:− > chr7:50571757:− | 9 |
| EVGPICNK | | |
| GDPGIDAR | chr21:43290029:− > chr21:43280481:− | 9 |
| MAFSGSQAPYLSPDPK | chr17:25958330:+ > chr17:25965125:+ | 9 |
| TIAQGNLSNTDVQAAKRSCSYL | chr16:21987564:+ > chr16:21988399:+ | 9 |
| MQENTLRAPTDSTLWHPPHYK | chr14:60442915:+ > chr14:60443943:+ | 9 |
| DQPGQHGPGMRPPLGLR | chr1:222838358:+ > chr1:222838651:+ | 9 |
| EAPTYITWTIRR | chr1:51218110:− > chr1:51210447:− | 9 |
| DLVGGYTPGTPYK | chr2:131824664:− > chr2:131813268:− | 9 |
| GLNEAIFMIPTNPPPTFR | chr8:99648370:− > chr8:99608397:− | 9 |
| DAAALLLANEAR | chr6:110774731:− > chr6:110768193:− | 9 |
| ATSLWYFMAAQAK | chr2:42615589:− > chr2:42580483:− | 9 |
| MDLVQVSGSLGSPVLSCK | chr10:104231153:+ > chr10:104231667:+ | 9 |
| QHQEVTLYGLK | chr15:99433697:+ > chr15:99434554:+ | 9 |
| LVSLSQYWEK | chr18:74574245:+ > chr18:74580641:+ | 9 |
| TGTTPVYSSQHEQHR | chr1:178806756:+ > chr1:178846633:+ | 9 |
| QATGMDLEVIILSDITQK | chr6:129786434:+ > chr6:129788349:+ | 9 |
| LLALLDVLK | chr1:22263648:− > chr1:22224962:− | 9 |
| GIGYPAWAMK | chr2:131840150:− > chr2:131829742:− | 9 |
| MESQLQIEETTLLR | chr16:4667729:+ > chr16:4700366:+ | 9 |
| GLLGLAGMRPELR | chr3:40555431:+ > chr3:40557351:+ | 9 |
| GQEMETILANMISR | chr9:96211969:− > chr9:96209979:− | 9 |
| ICRPGAAAAARHPGSPGGQLAPR | chr16:29692733:+ > chr16:29705985:+ | 9 |
| ELGVTAGGLPTR | chr12:42795298:+ > chr12:42835117:+ | 9 |
| GNLDTKTPGMPK | chr10:12280484:+ > chr10:12301986:+ | 9 |
| LLELQGLPLMPSR | chr19:53119971:− > chr19:53097557:− | 9 |
| AGGSLEAGGK | chr7:44883388:− > chr7:44882953:− | 9 |
| EGDVVLYCSSATFCATSQSGTGE | chr2:219296440:+ > chr2:219296580:+ | 9 |
| VR | | |
| STNDELDELR | chr18:8708134:+ > chr18:8718422:+ | 9 |
| INSAHLQTLSAGK | chr15:49600974:+ > chr15:49611801:+ | 9 |
| MGLSPASAPRPQTCFSLSTTK | chr17:17735071:− > chr17:17723835:− | 9 |
| MLADRSMSSSLSASQLHTVNMR | chr17:38177572:− > chr17:38176606:− | 9 |
| GTQVFVTGYTPWQR | chr20:1532337:− > chr20:1530245:− | 9 |
| METDLECSDSR | chr5:68551355:+ > chr5:68551980:+ | 9 |
| TLLPGTLR | chr7:129915476:+ > chr7:129916468:+ | 9 |
| WLMPVIPALGR | chr1:160283437:− > chr1:160282957:− | 9 |
| LLGAEIAPLHSSLDGLS | chr9:97214855:+ > chr9:97216240:+ | 9 |
| DPAIALQPGPQSER | chr15:79168112:+ > chr15:79170555:+ | 9 |
| MAHTLQYFK | chr8:72942008:+ > chr8:72964774:+ | 9 |
| MAHTLQYFKSSQHQR | chr8:72942008:+ > chr8:72964774:+ | 9 |
| MISAFPTEGQR | chr11:74830434:+ > chr11:74873700:+ | 9 |
| MDFMNAEILLK | chr19:49834874:− > chr19:49797810:− | 9 |
| SEEPLRPAAAPSENALR | chr9:119602892:− > chr9:119583062:− | 9 |
| TSPVAGPALPPNYK | chr13:45576476:+ > chr13:45578440:+ | 9 |
| EYLEYEDTAQR | chr2:113956803:+ > chr2:113966557:+ | 9 |
| QNLHEIGLWR | chr12:42835231:+ > chr12:42836287:+ | 9 |
| AGGSLEAK | chr19:6365650:+ > chr19:6366269:+ | 9 |
| AMSQINFFTNDPASAIGEYEDLR | chr10:118750845:− > chr10:118738819:− | 9 |
| LELALLAPQACR | chr8:53483830:− > chr8:53455005:− | 9 |
| SLVLEGFLINSKPENACEPI | chr3:149581921:+ > chr3:149589816:+ | 9 |
| TVTDSTLWIASSPGGLSPADCGAS | chr3:77121427:+ > chr3:77147165:+ | 9 |
| FR | | |
| IASTQEADVVVAR | chr5:94876747:− > chr5:94876534:− | 9 |
| LLRELQVMYGMLVFTLVLR | chr11:76708271:+ > chr11:76709807:+ | 9 |

The identification of peptides overlapping splicing regions is less sensitive likely due to 1) the lower abundance in the whole proteome of the sequence of interest compared to a non-spliced region and 2) splicing motifs code in around 40% of the cases for a lysine (K) or arginine (R) amino acids, which are also the cleavage sites for trypsin (i.e.: the enzyme used for MS sample processing). Therefore, the above-mentioned protocol probably leads to an underestimated of the presence of JET-derived proteins in tumor samples and cell lines.

It has been then demonstrated that tumor-specific JETs were indeed translated. Focusing on lung tumors, lung tumor-specific JETs predicted in TCGA and CCLE projects were in silico translated and interrogated against 2 different mass spectrometry output datasets: Nusinow et al. 2020 (CCLE proteomics dataset) and Stewart et al. 2019 (lung primary tumours proteomics dataset). This analysis provided information about JET-derived proteins not only in the context of a cell line, but also in cancer human samples. In cell lines, we were able to identify 221 peptides overlapping the splicing site of 206 JET-derived proteins (Table 6).

TABLE 6

| Sequence | chimeric_Tx_Id | Recurrence |
|---|---|---|
| HTDLQATAIGK | chr12: 104824437: +: Charlie6 > chr12: 104995684: | 108 |
| | +: ENST00000546689 | |
| YISLIYTNYAEGK | chr11: 67352712: +: ENST00000398603 > chr11: | 81 |
| | 67361105: +: LTR22A | |
| AQWLTPVIPALWEAEAGE | chr1: 36637320: +: AluSp > chr1: 36638065: +: | 45 |
| SPERPR | ENST00000530729 | |
| MILSSIR | chr6: 144066592: +: L3 > chr6: 144070122: +: | 45 |
| | ENST00000427704 | |
| MGISIVK | chr8: 74872000: − | 36 |
| | : ENST00000520242 > chr8: 74871067: −: Tigger3b | |
| LLINRMVTL | chr5: 52197198: +: MLT1D > chr5: 52201593: +: | 36 |
| | ENST00000282588 | |
| MGAVAHTCNPSTLGGQGK | chr12: 104645612: +: AluSc > chr12: 104651797: +: | 36 |
| | ENST00000429002 | |
| ALVNLSFVTGVSSMTFMM | chr2: 270006: +: MER39 > chr2: 271866: +: | 36 |
| GNICR | ENST00000442386 | |
| APVSDEK | chr7: 130069134: −: L3 > chr7: 130067859: − | 27 |
| | : ENST00000541543 | |
| MNSLSIR | chr9: 128638776: +: Tigger2a > chr9: | 27 |
| | 128677965: +:ENST00000491787 | |
| ALELGVVSQPSVYR | chr1: 223852296: −: MIRb > chr1: 223842101: − | 27 |
| | : ENST00000419193 | |
| MGAVTHTCNPSTLEGR | chr2: 207009280: −: AluJo > chr2: 207008856: − | 27 |
| | : ENST00000432169 | |
| ELLHGTEGGEHR | chr17: 27441216: −: L3 > chr17: 27441115: − | 27 |
| | ENST00000531253 | |
| VAASSPIMRK | chr11: 71728382: −: MIR > chr11: 71727576: − | 18 |
| | : ENST00000358965 | |
| TITLEVEPR | chr17: 16285735: +: ENST00000535788 > chr17: | 18 |
| | 16300446: +: MER4B | |
| MGAVAHTCNPSTLEGQDC | chr3: 185370866: −: AluSx1 > chr3: 185369956: − | 18 |
| PCGRPRR | : ENST00000421047 | |
| EAEEPSDNGPLFTELK | chr14: 97307963: +: AluSx > chr14: 97312432: +: | 18 |
| | ENST00000216639 | |
| LQDSTGSWEK | chrX: 40570189: −: MER8 > chrX: 40569381: − | 18 |
| | : ENST00000324817 | |
| GLPSHAELIR | chr12: 16168469: +: THE1D > chr12: 16185476: +: | 18 |
| | ENST00000531803 | |
| CNPSTLGDQAEK | chr2: 231040909: −: AluJb > chr2: 231037675: − | 18 |
| | : ENST00000258381 | |
| QLQSANYGHMQASCLSIK | chr12: 52639328: +: ENST00000331817 > chr12: | 18 |
| | 52645561: +: MER58A | |
| AHLNERNTLQEENK | chr18: 20537359: +: L2c > chr18: 20555116: +: | 18 |
| | ENST00000582354 | |
| DLYANTVMSVELNAASGT | chr7: 5567728: −: | 18 |
| R | ENST00000331789 > chr7: 5522250: −: MIRb | |
| TEEMPQLNFGMAVLYGG | chr3: 107447807: +: ENST00000415149 > chr3: | 18 |
| MCL | 107448789: +: AluJr | |
| ENLPSIQPGMVLQAVAHL | chr5: 140764890: +: ENST00000518325 > chr5: | 18 |
| HGR | 140765965: +: BC200 | |
| LLNATHQIGCQYTHR | chr1: 160314616: +: ENST00000421914 > chr1: | 18 |
| | 230653304: +: MLT1B | |
| MAVVAHACNLNSFGGQG | chr3: 49096310: −: AluSp > chr3: 49065271: −: | 18 |
| PAWFLR | ENST00000429182 | |
| MVWVGTIGSGPK | chr17: 59856034: −: | 18 |
| | MamRep1161 > chr17: 59853923: − | |
| | ENST00000259008 | |
| NLLDIDAPVTGWQELK | chr4: 102117073: −: | 18 |
| | ENST00000525819 > chr4: 102104428: −: MLT1J | |
| VISAFPSELQSQCTK | chr6: 100379769: −: L1PA3 > chr6: 100369131: −: | 18 |
| | ENST00000281806 | |
| MACNMGLEKGEK | chr5: 179238682: +: MER61- | 18 |
| | int > chr5: 179250858: +: ENST00000504627 | |
| MLDNLMYDYELNWVR | chr14: 106801819: −: LIME2 > chr14: 106518589: − | 18 |
| | : ENST00000390598 | |
| AKIAPAEGPDVSER | chr3: 185370866: −: AluSx1 > chr3: 185369956: − | 18 |
| | : ENST00000346192 | |
| MVLLVR | chr2: 189861722: +: (TA)n > chr2: 189861891: +: | 18 |
| | ENST00000304636 | |
| VGGSRVLGGDQ | chr6: 30690007: +: AluSc > chr6: 30690314: +: | 18 |
| | ENST00000327892 | |
| NTQLTMWLPQLIWTK | chr7: 127304880: +: MER21C > chr7: 127347646: + | 18 |
| | : ENST00000354725 | |
| MCQKEVMEQSAGR | chr2: 133425973: −: | 18 |
| | ENST00000345008 > chr2: 133381838: −MLT1B | |
| MAQWLMPVIPALWEAQIH | chr2: 224652913: −: AluSz > chr2: 224642586: − | 9 |
| FILLFSR | : ENST00000396653 | |
| LLSSSMHTLQTK | chrX: 38146409: +: CT−rich > chrX: 38146366: − | 9 |
| | : ENST00000378505 | |
| QQMLVCFVNLDVNK | chr2: 228948057: −: LIPA8 > chr2: 228892259: − | 9 |
| | : ENST00000344657 | |
| MASTISAGCGGGACGPSYS | chr20: 62284680: − | 9 |
| GG | : ENST00000370053 > chr20: 62277869: −: AluY | |
| LLDEAAGEGEAAGR | chr15: 83103156: +: ENST00000358583 > chr15: | 9 |
| | 83193188: +: GA−rich | |
| TSPVAGPALPPNYK | chr13: 45576476: +: 7SLRNA > chr13: 45578440: + | 9 |
| | : ENST00000361121 | |
| LSTVVDADEIIVLDQEK | chrX: 74280058: − | 9 |
| | : ENST00000529949 > chrX: 74267940: −: L1MB8 | |
| MDFMNAEILLK | chr19: 49834874: −: MLT1J2 > chr19: 49797810: − | 9 |
| | : ENST00000335875 | |
| MATVAHTSLATVEDFQIRP | chr2: 37520065: −: AluJb > chr2: 37518142: − | 9 |
| HTLYVHSYK | : ENST00000443187 | |
| METQADLVSQEPQALLDS | chr7: 99674926: − | 9 |
| ALREAEAGR | : ENST00000428683 > chr7: 99674180: −: AluSc8 | |
| LLITAEDQSMWIQS | chr13: 113897987: +: L1MC5 > chr13: 113898724: | 9 |
| | +: ENST00000326335 | |
| LADLFGWSQLIYNHITEYK | chr4: 2886393: +: ENST00000398129 > chr4: | 9 |
| | 2888303: +: L2a | |
| LLHHQLHMMLDK | chr1: 145474732: +: ENST00000323397 > chr1: | 9 |
| | 145476653: +: MIR3 | |
| LSPLPSNSCVTWGGYR | chr7: 7498396: −: MIRb > chr7: 7495743: − | 9 |
| | : ENST00000399429 | |
| MILESTYHNTLHIAMK | chr6: 17835789: −: MIR > chr6: 17834302: − | 9 |
| | : ENST00000378814 | |
| MQENTLRAPTDSTLWHPP | chr14: 60442915: +: MER1A > chr14: 60443943: +: | 9 |
| HYK | ENST00000254271 | |
| MMVNCHAHFWVSTYLSP | chr8: 74209664: +: ENST00000521928 > chr8: | 9 |
| HLL | 74216833: +: L2 | |
| SQVMDVDTLY | chr3: 98618227: −: AluSc8 > chr3: 98600611 | 9 |
| | : −: ENST00000326857 | |
| TDDSLGLGLK | chr11: 14737526: +: L1PA2 > chr11: 14793483: +: | 9 |
| | ENST00000282096 | |
| GHEFETSLANMVIQLLK | chr7: 154793407: −: AluSq > chr7: 154790494: − | 9 |
| | : ENST00000457196 | |
| MDGQMMDTGVDK | chr4: 2453586: +: (TGGA)n > chr4: 2453869: +: | 9 |
| | ENST00000506607 | |
| HLEDYMEHTPPPR | chr2: 95476942: +: MIR > chr2: 95540521: +: | 9 |
| | ENST00000295201 | |
| IFTYQICK | chr15: 72463983: −: L1M5 > chr15: 72462280: − | 9 |
| | : ENST00000564129 | |
| TENLGSILSWWHNK | chr3: 72442890: −: L1MC4a > chr3: 72428578: − | 9 |
| | : ENST00000477973 | |
| MGGATSPLLSSAK | chr17: 56588640: −: MIRb > chr17: 56586202: − | 9 |
| | : ENST00000579925 | |
| MISAFPTEGQR | chr11: 74830434: +: L1PA4 > chr11: 74873700: +: | 9 |
| | ENST00000289575 | |
| TFTMIHFHLITR | chr12: 4948527: +: Tigger2a > chr12: 4959904: +: | 9 |
| | ENST00000542998 | |
| LLPPCQPGSSLSSK | chr16: 18565603: −: MIR > chr16: 18540905: | 9 |
| | −: ENST00000569051 | |
| GLFSQAEIR | chr6: 123549550: −: THE1C > chr6: 123545276: | 9 |
| | −: ENST00000398178 | |
| MAALTIATGLAVD | chr11: 77850610: −: | 9 |
| | ENST00000530454 > chr11: 77848691: −: Tigger1 | |
| MYMAHGLTQSTK | chr5: 153529506: +: L2c > chr5: 153674376: +: | 9 |
| | ENST00000425427 | |
| EFETSLGLWQAMR | chr9: 15573873: +: AluJo > chr9: 15578847: +: | 9 |
| | ENST00000535968 | |
| TAELPKPSISSNNSNPK | chr19: 42217314: +: Tigger4a > chr19: | 9 |
| | 42311149: +: ENST00000415495 | |
| VQDHPGQHGEAIGK | chrX: 47312190: −: AluSx > chrX: 47308873: − | 9 |
| | : ENST00000432977 | |
| TSLGNMINDK | chr15: 64365987: −: AluJr4 > chr15: 64365216: − | 9 |
| | : ENST00000300030 | |
| MLPPFVLAVTMGLSVVK | chr3: 15058779: +: L2c > chr3: 15062260: +: | 9 |
| | ENST00000323373 | |
| QIGCYQGFAFAWK | chr11: 69488615: −: AluJo > chr11: 69486587: − | 9 |
| | : ENST00000542341 | |
| SQGSGLNFIDLMVR | chr16: 77786496: +: MIR3 > chr16: 77850818: +: | 9 |
| | ENST00000302536 | |
| AWEAEIMVKYLLFMWEIK | chr2: 177179106: +: AluSz6 > chr2: 177191553: +: | 9 |
| | ENST00000249442 | |
| ATVWLGSGDEK | chr16: 50651682: +: MER91A > chr16: 50655550: + | 9 |
| | : ENST00000268459 | |
| DNTDSVVFEDVAVNFTQE | chr19: 12635610: −: L1MB3 > chr19: 12577664: − | 9 |
| EWALLGPSQK | : ENST00000598753 | |
| QSLSEELK | chr2: 211453855: +: LTR16C > chr2: 211454830: +: | 9 |
| | ENST00000430249 | |
| AAAVAAKAEAVSPAK | chr19: 39833216: +: (CGG)n > chr19: 39866290: +: | 9 |
| | ENST00000598913 | |
| KLILCLNDCK | chr12: 104518408: −: MER33 > chr12: 104517201: − | 9 |
| | : ENST00000240055 | |
| TGAQPPATMPLNVSFTNR | chr8: 128749923: +: G-rich > chr8: | 9 |
| | 128750494: +: ENST00000377970 | |
| DLEFETSLANK | chr10: 103896914: +: AluSx1 > chr10: 103897607: | 9 |
| | +: ENST00000278070 | |
| LSSQHLGGQEPEQLPEKR | chr1: 19410263: −: AluJo > chr1: 19408067: − | 9 |
| | : ENST00000375224 | |
| TPEVEATVPLQK | chr22: 42085308: +: AluSx1 > chr22: 42089467: +: | 9 |
| | ENST00000402966 | |
| ISQLQIWDTAGQER | chr3: 128828867: −: AluJo > chr3: 128814012: − | 9 |
| | : ENST00000457077 | |
| VILIFFPECYAK | chr22: 31947110: +: L2 > chr22: 31952928: +: | 9 |
| | ENST00000450787 | |
| IRDSEELIGR | chr15: 79649214: +: LIME4a > chr15: 79703742: +: | 9 |
| | ENST00000424155 | |
| MLAPENSDIGLAAWGR | chr20: 32888526: −: L2b > chr20: 32883391: − | 9 |
| | : ENST00000217426 | |
| QETPMAADEGSAEK | chr9: 140606077: +: L1MB7 > chr9: 140611078: +: | 9 |
| | ENST00000460843 | |
| MLADRSMSSSLSASQLHT | chr17: 38177572: −: MIRb > chr17: 38176606: − | 9 |
| VNMR | : ENST00000394126 | |
| LGVGSDSEVEER | chr12: 124157140: +: AluJo > chr12: 124158165: +: | 9 |
| | ENST00000303372 | |
| IQEAEVAVK | chr17: 48743773: +: AluSp > chr17: 48744915: +: | 9 |
| | ENST00000502426 | |
| IDMISCVWK | chr2: 223509604: −: MER44A > chr2: 223507626: − | 9 |
| | ENST00000281828 | |
| MGVVAHTCNPNTLGGQGI | chr11: 125450741: +: AluSx1 > chr11: 125451107: | 9 |
| EMHQR | +: ENST00000278903 | |
| EDQMSSWSISTSVQPSGR | chr1: 208254106: −: MIRb > chr1: 208252795: − | 9 |
| | : ENST00000367033 | |
| MLLTHWLNLADHWR | chrX: 63029083: −: MIR3 > chrX: 62944591: − | 9 |
| | : ENST00000253401 | |
| MEQFVQVYNWIDV | chr6: 39290104: − | 9 |
| | : ENST00000507712 > chr6: 39289934: −: MIR3 | |
| LLSSLFSEPTCLILVSPQ | chr17: 43026722: −: L2c > chr17: 43013726: − | 9 |
| | : ENST00000590129 | |
| TAQLPEQLMTWHSR | chr22: 37870550: − | 9 |
| | : ENST00000356998 > chr22: 37861756: −: L2a | |
| SYQLRPGTMIEWGNYWR | chr7: 56052571: +: ENST00000322090 > chr7: | 9 |
| | 56059164: +: AluSp | |
| QEDPLNLTCPSCGITFAPK | chr16: 4052808: −: AluSx > chr16: 4043511: − | 9 |
| | : ENST00000571467 | |
| TQEAEVANAASK | chr18: 19241710: −: AluSp > chr18: 19239304: − | 9 |
| | : ENST00000289119 | |
| ITSVVMVDEAHER | chr6: 30629093: −: AluY > chr6: 30628019: − | 9 |
| | : ENST00000376442 | |
| NIFSCISMGNFGGPGK | chr1: 92518215: +: ENST00000370383 > chr1: | 9 |
| | 92524420: +: LTR86B1 | |
| VISAFPSEVPGSSHSGVPDS | chr16: 21059883: −: L1PA2 > chr16: 21051265: − | 9 |
| GR | : ENST00000415178 | |
| DILMYMVWWCVPMMPAT | chr2: 225400245: − | 9 |
| RR | : ENST00000409096 > chr2: 225395962: −: AluSz | |
| GQEIQTILANTCIPR | chr1: 10478650: +: AluSc > chr1: 10478883: +: | 9 |
| | ENST00000270776 | |
| METDLECSDSR | chr5: 68551355: +: ENST00000502604 > chr5: | 9 |
| | 68551980: +: AluJo | |
| MLSFMRPPQPCFLYSLWN | chr9: 17365375: +: MSTA > chr9: 17366615: +: | 9 |
| | ENST00000380647 | |
| MLEKLEFEDEVPGP | chr7: 151329155: − | 9 |
| | : ENST00000493872 > chr7: 151297948: −: MIRb | |
| MELTIPGVGHSSGSLNEGK | chr2: 99224633: −: MIRb > chr2: 99220654: − | 9 |
| | : ENST00000328709 | |
| MASICPFLK | chr20: 49307663: − | 9 |
| | : ENST00000535356 > chr20: 49307455: −: MIR3 | |
| AFLAMIHSVSSMK | chr7: 151865652: −: UCON26 > chr7: 151864463: − | 9 |
| | : ENST00000355193 | |
| LSLSEDTDNSSLSPPPAK | chrX: 19072648: −: L1ME4a > chrX: 19058342: − | 9 |
| | : ENST00000357991 | |
| LHHWTPAWAAEK | chr17: 80441180: +: AluSx > chr17: 80443373: +: | 9 |
| | ENST00000457415 | |
| TISPFTGK | chr15: 29384448: +: MER58A > chr15: 29385278: + | 9 |
| | : ENST00000561069 | |
| MSAALMFDAVHVVVSAV | chr19: 42559834: −: L2a > chr19: 42558624: − | 9 |
| R | : ENST00000301218 | |
| MLLTTFVIHGR | chr17: 15939279: −: Tigger1 > chr17: 15938258: − | 9 |
| | : ENST00000395851 | |
| MLLTTFVIHGRR | chr17: 15939279: −: Tigger1 > chr17: 15938258: − | 9 |
| | : ENST00000395851 | |
| EYIVPMLTGLQPIMIIQ | chr5: 139864383: +: L1ME3A > chr5: 139864706: + | 9 |
| | : ENST00000421134 | |
| PQNSTASVSEAER | chr1: 21588222: −: L2c > chr1: 21586885: − | 9 |
| | : ENST00000473505 | |
| SVSTIFLTCSR | chr10: 1100108: −: Tigger1 > chr10: 1090111: − | 9 |
| | : ENST00000381344 | |
| MVILESMVSLTQELCPVA | chr14: 24530511: +: MIRc > chr14: 24530706: +: | 9 |
| MR | ENST00000342740 | |
| VSTEEEK | chr15: 51201329: +: MIRb > chr15: 51204275: +: | 9 |
| | ENST00000558439 | |
| MAHLLPNVSEETVSPTR | chr9: 123334133: −: L2b > chr9: 123330666: − | 9 |
| | : ENST00000481266 | |
| MGTVADTCDPSTLGGGVC | chr2: 201751637: −: AluJo > chr2: 201750495: − | 9 |
| DTAYR | : ENST00000415562 | |
| MLQPSQEVGTISK | chr4: 157831776: −: L1MD2 > chr4: 157782641: − | 9 |
| | : ENST00000274071 | |
| TQSLGVDEVTIVNILTNRS | chr15: 60689166: −: MIRc > chr15: 60656722: − | 9 |
| N | : ENST00000559370 | |
| MPGPLRSLEMAR | chr19: 20278169: +: ENST00000597083 > chr19: | 9 |
| | 20281106: +: AluSx1 | |
| VMGL VIPDVPK | chr13: 96595382: −: L1ME3F > chr13: 96592345: − | 9 |
| | : ENST00000376747 | |
| LHHCTPAWLTER | chr9: 33878600: +: AluJb > chr9: 33886879: +: | 9 |
| | ENST00000263228 | |
| MLSTSWTLPR | chr20: 30459529: −: L2a > chr20: 30457400: − | 9 |
| | : ENST00000278979 | |
| LAPALLSAMIFAVLWSTR | chr17: 64403546: +: MSTA > chr17: 64492319: +: | 9 |
| | ENST00000284384 | |
| NGISGQVLK | chr2: 238991995: +: ENST00000433750 > chr2: | 9 |
| | 238992535: +: L1MDa | |
| MQLDQICISFDEGK | chr22: 50947090: +: Charlie9 > chr22: | 9 |
| | 50954876: +: ENST00000299821 | |
| MGAWLTPDTGVGK | chr18: 9766693: +: AluJr4 > chr18: 9775275: +: | 9 |
| | ENST00000578921 | |
| STNDELDELR | chr18: 8708134: +: L2c > chr18: 8718422: +: | 9 |
| | ENST00000306329 | |
| EAEAGESLKPGR | chr17: 421135: −: AluJr > chr17: 420873: | 9 |
| | −: ENST00000574029 | |
| SQILGMQSLALVAQSGVQ | chr11: 17098209: −: FRAM > chr11: 17098094: −: | 9 |
| WR | ENST00000533969 | |
| WQIYSGPLPTSPGAACLPC | chr16: 57483993: +: L1ME2z > chr16: 57484952: +: | 9 |
| FSCGAK | ENST00000262507 | |
| EYLEYEDTAQR | chr2: 113956803: +: ENST00000245796 > chr2: | 9 |
| | 113966557: +: L4 | |
| SSGAQGEYAGLATIR | chr9: 6566281: −: L1MC5 > chr9: 6565429: −: | 9 |
| | ENST00000321612 | |
| AGGSGSVVIWTVPTGSWQ | chr15: 75629457: +: AluJr > chr15: 75630403: +: | 9 |
| K | ENST00000564815 | |
| VNGYPNYLIR | chr14: 45702609: −: L2a > chr14: 45702023: −: | 9 |
| | ENST00000453142 | |
| MASAWEGGAWR | chr14: 105195833: +: (GCGTG)n > chr14: 1051961 | 9 |
| | 66: +: ENST00000555486 | |
| CMCLEYPAVVEFAPFQK | chr13: 115049839: +: (TG)n > chr13: 115051777: +: | 9 |
| | ENST00000375299 | |
| TSLANMVGMFK | chr6: 136924515: −: AluSx1 > chr6: 136923117: − | 9 |
| | : ENST00000359015 | |
| FSEASVAMLPVQLWNR | chr6: 147981955: +: MSTA > chr6: 148058453: +: | 9 |
| | ENST00000566741 | |
| VETLCATLWGFGK | chr10: 102711867: +: AluSz > chr10: 102716208: +: | 9 |
| | ENST00000370269 | |
| MDLVQVSGSLGSPVLSCK | chr10: 104231153: +: ENST00000366277 > chr10: | 9 |
| | 104231667: +: MIR3 | |
| MVAGPTYLGDLCPPK | chr14: 94523351: −: MIR > chr14: 94521530: − | 9 |
| | : ENST00000330836 | |
| HCHALMPK | chr12: 15701267: +: L1MC4a > chr12: 15702028: +: | 9 |
| | ENST00000535311 | |
| ASGSQGMFK | chr8: 95855580: +: AluSx3 > chr8: 95861690: +: | 9 |
| | ENST00000343161 | |
| IVESGDTPK | chr2: 25505735: −: Charlie24 > chr2: 25505580: − | 9 |
| | : ENST00000406659 | |
| ASGSLEGPFVTWR | chr19: 15297447: −: AluSx > chr19: 15296490: − | 9 |
| | : ENST00000263388 | |
| ENTMAENELQV | chr10: 112572705: +: ENST00000369519 > chr10: | 9 |
| | 112576399: +: L2a | |
| TVTVPAQELK | chr9: 128421519: −: MIR > chr9: 128420078: − | 9 |
| | : ENST00000373496 | |
| NLQLSAHLLTSSLK | chr9: 112168615: −: MER102a > chr9: 112166888: − | 9 |
| | : ENST00000374541 | |
| NLDLLEGMESTQMPK | chr13: 38183213: −: LIMEf > chr13: 38171419: − | 9 |
| | : ENST00000541481 | |
| LDGHAGDLDIEDDMK | chr14: 62187825: +: AluSg4 > chr14: 62188227: +: | 9 |
| | ENST00000394997 | |
| MTKLLSPIR | chr22: 32787416: −: AluSx1 > chr22: 32784086: − | 9 |
| | : ENST00000216038 | |
| YEEEIRTCR | chr6: 24551662: − | 9 |
| | : ENST00000543707 > chr6: 24541573: −: MLT1B | |
| QAWRLMPVIPAHWEAEV | chr7: 156789005: −: AluJo > chr7: 156786875: − | 9 |
| K | : ENST00000469500 | |
| AEIALLHSSLGNREK | chrX: 114878268: +: AluSx > chrX: 114879341: +: | 9 |
| | ENST00000537301 | |
| TFWTNDDPGLEVLVPEGG | chr6: 134528494: − | 9 |
| VLPETQQ | : ENST00000367858 > chr6: 134508473: −: ERVL- | |
| | B4-int | |
| QEAEIPGANLNVEILSSCR | chr1: 117629144: +: ENST00000369466 > chr1: | 9 |
| | 117630672: +: L2c | |
| LNEGASGLDGKPGSR | chr4: 109762023: −: MIRb > chr4: 109755546: − | 9 |
| | : ENST00000494183 | |
| MESQLQIEETTLLR | chr16: 4667729: +: AluJb > chr16: 4700366: +: | 9 |
| | ENST00000588994 | |
| MSDPVFLSLSLLVCETGDH | chr9: 20361021: −: MIR > chr9: 20360839: − | 9 |
| EKRNGR | : ENST00000380338 | |
| ICHFLSVTGSLLR | chr2: 74071187: +: MIR > chr2: 74071940: +: | 9 |
| | ENST00000424659 | |
| MSSINPNTSATWK | chr14: 90416441: − | 9 |
| | : ENST00000267544 > chr14: 90416136: −: THE1B | |
| IILANTFSK | chr3: 185328562: +: AluY > chr3: 185329439: +: | 9 |
| | ENST00000545472 | |
| FCRLLNGLGEEIK | chr10: 28247767: −: MIRb > chr10: 28233891: − | 9 |
| | : ENST00000537576 | |
| AQEFETSLGNMFR | chr12: 95596369: −: FLAMC > chr12: 95566520: | 9 |
| | −: ENST00000451107 | |
| MSDQEKGPIVSSGVR | chr2: 203103163: −: | 9 |
| | ENST00000392244 > chr2: 203098881: −: | |
| | Charlie4z | |
| TNYAASSYLSLTPEQWK | chr22: 23204348: +: L1MA4A > chr22: 23248706: | 9 |
| | +: ENST00000390325 | |
| TSLGNMLTADPR | chr14: 35778674: +: AluSx > chr14: 35779946: +: | 9 |
| | ENST00000556506 | |
| SPVTMEQLICK | chr2: 90074903: +: MER70− | 9 |
| | int > chr2: 90139454: +: ENST00000492446 | |
| GAGPAVPGGPVPAGATLT | chr17: 2627917: +: GCrich > chr14: 106330469: − | 9 |
| TGAR | : ENST00000461719 | |
| GQEFETSLANMETPGR | chr16: 3718260: −: AluSx > chr16: 3716119: − | 9 |
| | : ENST00000576335 | |
| FVDVTECQACSANSR | chr22: 21567354: − | 9 |
| | : ENST00000424627 > chr22: 21564141: −: L2b | |
| TELNIVPVPR | chr19: 41438041: +: L2b > chr19: 41509906: +: | 9 |
| | ENST00000330446 | |
| MTLATDGTADMFGIR | chr3: 4927407: − | 9 |
| | : ENST00000441894 > chr3: 4898429: −: MLT1D | |
| MELSAQFAATTTTPPRPLG | chr11: 58943356: +: MIR3 > chr11: 58949212: +: | 9 |
| R | ENST00000227451 | |
| EAIALCHFFFNISGILLWYP | chr4: 25676447: +: L3 > chr4: 25677757: +: | 9 |
| IPFTR | ENST00000503434 | |
| DAAALLLANEAR | chr6: 110774731: −: LTR40c > chr6: 110768193: − | 9 |
| | : ENST00000434949 | |
| MELTIPGVGK | chr2: 99224656: −: MIRb > chr2: 99220650: − | 9 |
| | : ENST00000328709 | |
| METPAQLLFLLLLWLPAE | chr2: 89442543: − | 9 |
| AGGIMR | : ENST00000492167 > chr2: 89438968: −: AluYb8 | |
| MGAVTHIWNPRTLGGQDP | chr17: 40046175: −: AluSq2 > chr17: 40043956: − | 9 |
| DHR | : ENST00000353196 | |
| LTMGSTGCDRPLDK | chr14: 106717516: −: L1MC > chr14: 106641752: − | 9 |
| | : ENST00000390605 | |
| WGGQAPPGQPPRPGGR | chr19: 39346300: −: SVAD > chr19: 39338074: − | 9 |
| | : ENST00000601449 | |
| LRQEQLLEPRMQR | chr19: 50809471: +: AluSp > chr19: 50812982: +: | 9 |
| | ENST00000262269 | |
| TLEVEGK | chr12: 76520433: −: AluSx1 > chr12: 76443026: − | 9 |
| | ENST00000431879 | |
| NLEASWATR | chr16: 14738989: +: AluJr > chr16: 14742245: +: | 9 |
| | ENST00000570219 | |
| ELYPGQFKPPICLL | chr19: 39380329: − | 9 |
| | : ENST00000358931 > chr19: 39380152: −: MIR | |
| MLVSASQDGK | chr7: 100274628: +: AluJo > chr7: 100274975: +: | 9 |
| | ENST00000412215 | |
| AGFYEQMNGPVAG | chr8: 144745326: −: AluSz > chr8: 144668944: − | 9 |
| | : ENST00000531931 | |
| MLGTRPHGR | chr11: 73445789: −: MER1B > chr11: 73441868: − | 9 |
| | ENST00000336083 | |
| LAVNSPPVEPPEQHSR | chr1: 53346501: +: MLT1E1A > chr1: 53347143: +: | 9 |
| | ENST00000371532 | |
| MIVMNDQMDSSLK | chr10: 64976967: − | 9 |
| | : ENST00000402544 > chr10: 64445839: −: L2c | |
| MGTVAHACNTSTLGGQEH | chr22: 21577153: −: AluJb > chr22: 21104282: − | 9 |
| CEGLR | : ENST00000255882 | |
| MSLCILSMK | chr19: 9939645: +: MIRb > chr19: 9940641: +: | 9 |
| | ENST00000586895 | |
| IPTVTPGAASSC | chr17: 62544114: −: MER5B > chr17: 62499650: − | 9 |
| | ENST00000450599 | |
| TITDAELPIYR | chr14: 51362421: +: HERVH- | 9 |
| | int > chr14: 51368547: +: ENST00000353130 | |
| GLEWVSQISESTNSL | chr14: 107048816: − | 9 |
| | ENST00000390627 > chr14: 107047602: − | |
| | : LTR13 | |
| VAVSVLSPGK | chr11: 69063184: +: ENST00000308946 > chr11: | 9 |
| | 69084301: +: MIRb | |
| GQVIGEIDEETDSALDL | chr7: 117829683: +: AluSq2 > chr7: 117831971: +: | 9 |
| GNIR | ENST00000422760 | |
| DQPGQHGPGMRPPLGLR | chr1: 222838358: +: AluJb > chr1: 222838651: +: | 9 |
| | ENST00000344441 | |
| VILVNLGSSAEWVFQR | chr9: 123165587: −: L2c > chr9: 123165349: − | 9 |
| | : ENST00000416449 | |
| TTALQPGHSDLVTAR | chr9: 127988405: +: AluSx > chr9: 127990189: +: | 9 |
| | ENST00000259460 | |

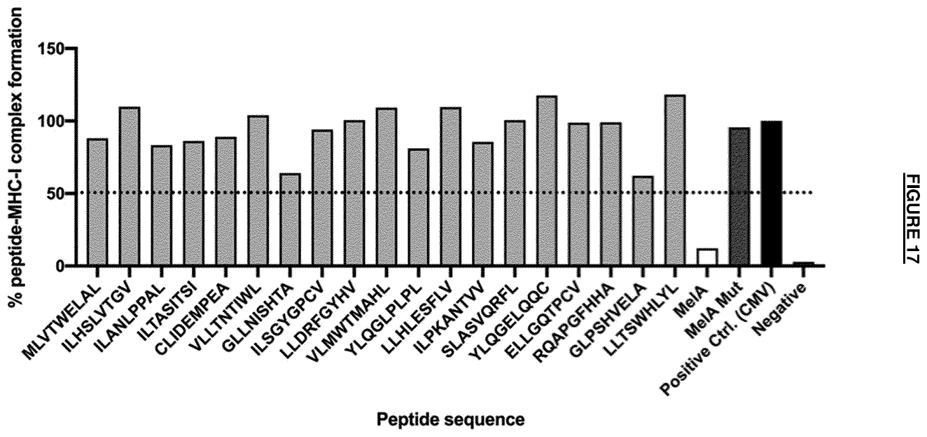

In lung primary tumors, 167 JET-derived proteins were identified (FIG. 16A and Table 7).

TABLE 7

| Sequence | chimeric_id_all | Recurrence |
|---|---|---|
| LSVLSQPK | chr2: 134035154: +: L1M5 > chr22: 23248512: +: ENST00000390325 | 100 |
| SNLNTGVPS | chr2: 90214357: +: L1ME3 > chr2: 90249278: +: ENST00000468879, | 99 |
| R | chr2: 90214357: +: L1ME3 > chr2: 90260131: +: ENST00000471857 | |
| LDSQYVER | chr19: 18432008: −: L2a > chr19: 18426875: −: ENST00000593829 | 81 |
| DIVRSDLEG | chr22: 45565558: +: LTR13A > chr22: 45566869: +: ENST00000434760 | 71 |
| KR | | |
| AEIAPLNSSL | chr19: 8529495: +: AluSq2 > chr19: 8530208: +: ENST00000348943 | 46 |
| GR | | |
| TFTGGAVIV | chr20: 44410315: −: MER68 > chr20: 44404241: −: ENST00000337205 | 35 |
| QK | | |
| VETSDEEIHK | chr1: 226016613: +: ENST00000445856 > chr1: 226018885: +: MER58B, | 32 |
| K | chr1: 226016613: +: ENST00000445856 > chr1: 226018885: +: MER58B | |
| SQLLERPRQ | chr19: 17387073: +: AluSq2 > chr19: 17387304: +: ENST00000596335, | 31 |
| ENRLNLGAS | chr19: 17387073: +: AluSq2 > chr19: 17387304: +: ENST00000598188, | |
| R | chr19: 17387073: +: AluSq2 > chr19: 17387304: +: ENST00000599474, | |
| | chr19: 17387073: +: AluSq2 > chr19: 17387304: +: ENST00000601938, | |
| | chr19: 17387073: +: AluSq2 > chr19: 17387304: +: ENST00000602066 | |
| LSLGSRIDQI | chr3: 4867430: −: ENST00000449914 > chr3: 4859235: −: L1MC4 | 30 |
| NISR | | |
| NGKFPSLLT | chr6: 35419093: +: L2b > chr6: 35438040: +: ENST00000322203 | 26 |
| HNENMVAK | | |
| ACLAEGR | chr3: 40501213: +: AluY > chr3: 40502924: +: ENST00000338970 | 25 |
| ILNCQKAM | chr9: 140458886: −: ENST00000277540 > chr9: 140458662: −: L2 | 24 |
| APSFR | | |
| TEFLSFMNT | chr1: 152006124: −: ENST00000271638 > chr1: 152005339: −: MIRc, | 23 |
| ELAAFTKK | chr1: 152006124: −: ENST00000271638 > chr1: 152005339: −: MIRc | |
| VLGVGSTR | chr1: 46675049: +: HAL1 > chr1: 46685371: +: ENST00000371980 | 21 |
| QNLQQAQC | chrX: 38015613: −: MER5A1 > chrX: 38013836: −: ENST00000432886, | 20 |
| GLDLR | chrX: 38015613: −: MER5A1 > chrX: 38013836: −: ENST00000432886, | |
| | chrX: 38015613: −: MER5A1 > chrX: 38013836: −: ENST00000432886 | |
| MRRPGQAS | chr9: 130213388: −: MIR3 > chr9: 130213083: −: ENST00000361436, | 15 |
| AFLGLSFFTL | chr9: 130213388: −: MIR3 > chr9: 130213083: −: ENST00000361436, | |
| YLR | chr9: 130213388: −: MIR3 > chr9: 130213083: −: ENST00000361436, | |
| | chr9: 130213388: −: MIR3 > chr9: 130213083: −: ENST00000536368, | |
| | chr9: 130213388: −: MIR3 > chr9: 130213083: −: ENST00000536368, | |
| | chr9: 130213388: −: MIR3 > chr9: 130213083: −: ENST00000536368 | |
| TQGGSMGN | chr2: 114668071: +: MER21B > chr2: 114670749: +: ENST00000263238, | 15 |
| SSSSR | chr2: 114668071: +: MER21B > chr2: 114670749: +: ENST00000263238, | |
| | chr2: 114668071: +: MER21B > chr2: 114670749: +: ENST00000415792, | |
| | chr2: 114668071: +: MER21B > chr2: 114670749: +: ENST00000415792, | |
| | chr2: 114668071: +: MER21B > chr2: 114670749: +: ENST00000446821, | |
| | chr2: 114668071: +: MER21B > chr2: 114670749: +: ENST00000446821 | |
| NLDNLPSRL | chr18: 66490653: +: MER51E > chr18: 66505943: +: ENST00000584775 | 14 |
| SIR | | |
| VVSKVEFVH | chr7: 156483036: −: Charlie7a > chr7: 156480885: −: ENST00000353442 | 14 |
| FLAFIK | | |
| LPRTGIGAP | chr7: 94018589: +: L2c > chr7: 94057721: +: ENST00000297268 | 14 |
| RTR | | |
| MGWMGEA | chr9: 130341201: −: ENST00000373314 > chr9: 130334967: −: MIRb | 12 |
| VR | | |
| MALSQCGP | chr19: 53754789: −: ENST00000333952 > chr19: 53750572: −: MER30 | 12 |
| GKPK | | |
| LLITTSAGGR | chr3: 194340620: −: L3 > chr3: 194337998: −: ENST00000392432 | 12 |
| LSCAASGFT | chr14: 106866613: −: ENST00000390618 > chr14: 106764783: −: AluJb | 12 |
| LR | | |
| ASQASVPPA | chr2: 112896307: +: ENST00000331203 > chr2: 112896389: +: C−rich | 11 |
| SLPCPSR | | |
| ASVTLIR | chr3: 44887389: +: MER65D > chr3: 44889477: +: ENST00000326047 | 11 |
| MNSLSIR | chr9: 128638776: +: Tigger2a > chr9: 128677965: +: ENST00000342287, | 11 |
| | chr9: 128638776: +: Tigger2a > chr9: 128677965: +: ENST00000373487, | |
| | chr9: 128638776: +: Tigger2a > chr9: 128677965: +: ENST00000373489, | |
| | chr9: 128638776: +: Tigger2a > chr9: 128677965: +: ENST00000428092, | |
| | chr9: 128638776: +: Tigger2a > chr9: 128677965: +: ENST00000491787 | |
| APVTGWQE | chr4: 102117073: −: ENST00000492351 > chr4: 102104428: −: MLT1J | 11 |
| LK | | |
| MEAYTPKLT | chr11: 18373993: +: ENST00000526630 > chr11: 18374659: +: AluSq2 | 11 |
| QLLK | | |
| SHLLGRLSQ | chr1: 234516671: +: AluSx > chr1: 234519469: +: ENST00000366615 | 11 |
| ENHWNLIK | | |
| GLEFETSLA | chr4: 25395102: +: AluSz6 > chr4: 25395461: +: ENST00000315368 | 8 |
| NMHGKK | | |
| TNVQQTTD | chr14: 31588916: −: ENST00000399332 > chr14: 31586842: −: L1ME4a, | 8 |
| LEIPPPGTV | chr14: 31588916: −: ENST00000399332 > chr14: 31586842: −: L1ME4a, | |
| GYCAVK | chr14: 31588916: −: ENST00000553957 > chr14: 31586842: −: L1ME4a, | |
| | chr14: 31588916: −: ENST00000553957 > chr14: 31586842: −: L1ME4a | |
| LARHGWAE | chr8: 54994449: −: AluSq2 > chr8: 54978373: −: ENST00000316963, | 8 |
| AFAGIRSSHI | chr8: 54994449: −: AluSq2 > chr8: 54978373: −: ENST00000343231, | |
| K | chr8: 54994449: −: AluSq2 > chr8: 54978373: −: ENST00000518546 | |
| MATVYFTR | chr19: 49497180: +: ENST00000595090 > chr19: 49497762: +: L2a | 8 |
| ATSQCK | | |
| LGGSSCLVA | chr20: 35116711: +: MIR > chr20: 35125108: +: ENST00000373907, | 8 |
| YKK | chr20: 35116711: +: MIR > chr20: 35125108: +: ENST00000373913 | |
| LLIYAASNG | chr2: 90199062: +: ENST00000390276 > chr2: 90207369: +: MER66−int, | 8 |
| HSGQQ | chr2: 90199062: +: ENST00000390276 > chr2: 90207369: +: MER66−int | |
| MAACLGLP | chr2: 172725191: −: ENST00000392592 > chr2: 172723793: −: SVA_D, | 8 |
| K | chr2: 172725191: −: ENST00000392592 > chr2: 172723793: −: SVA_D | |
| MSLGLSIHH | chr17: 37028428: +: MIR3 > chr17: 37034339: +: ENST00000318008 | 8 |
| LSDGSGIK | | |
| AEDTATSCR | chr14: 106573251: −: ENST00000390601 > chr14: 106558237: −: L1PB4 | 8 |
| AK | | |
| ISSEQLFCFT | chr19: 32948256: +: L4 > chr19: 32949006: +: ENST00000392250 | 7 |
| LINPK | | |
| AIALQPGQQ | chrX: 114878268: +: AluSx > chrX: 114879341: +: ENST00000355899, | 7 |
| RETR | chrX: 114878268: +: AluSx > chrX: 114879341: +: ENST00000497870 | |
| MGVVAHAC | chr7: 6508832: −: AluY > chr7: 6505954: −: ENST00000258739 | 7 |
| NSSTLGGQD | | |
| PLDLLHLPG | | |
| VR | | |
| MELTIPGEG | chr2: 99224660: −: MIRb > chr2: 99220654: −: ENST00000328709, | 7 |
| K | chr2: 99224660: −: MIRb > chr2: 99220654: −: ENST00000328709, | |
| | chr2: 99224660: −: MIRb > chr2: 99220654: −: ENST00000328709 | |
| MAAAALRD | chr19: 57999351: +: ENST00000354197 > chr19: 58024390: +: LTR3B | 7 |
| PAQSR | | |
| GPSPSGAEP | chr17: 43880005: +: Tigger12 > chr17: 43884376: +: ENST00000347197, | 6 |
| R | chr17: 43880005: +: Tigger12 > chr17: 43884376: +: ENST00000352855 | |
| LLSSSMHTL | chrX: 38146409: +: CT−rich > chrX: 38146366: −: ENST00000318842, | 4 |
| QTK | chrX: 38146409: +: CT−rich > chrX: 38146366: −: ENST00000378505, | |
| | chrX: 38146409: +: CT−rich > chrX: 38146366: −: ENST00000482855 | |
| SSRPAWPT | chr19: 55882935: −: AluSx > chr19: 55880309: −: ENST00000264563 | 4 |
| WCLPPGPG | | |
| R | | |
| MTDGGLAL | chr12: 110873462: −: AluSz > chr12: 110873022: −: ENST00000228825 | 4 |
| QFWNHFVC | chr22: 42077159: −: L1MC4a > chr22: 42076368: −: ENST00000355257 | 4 |
| IVEETEADV | | |
| NPK | | |
| GLILLEQDTA | chr9: 15619847: +: Tigger1 > chr9: 15623265: +: ENST00000297641, | 4 |
| VQNMHK | chr9: 15619847: +: Tigger1 > chr9: 15623265: +: ENST00000380701, | |
| | chr9: 15619847: +: Tigger1 > chr9: 15623265: +: ENST00000535968 | |
| LASVFQLQL | chr10: 29778636: −: AluJb > chr10: 29777685: −: ENST00000375400 | 4 |
| LR | | |
| DASKGVSLH | chrX: 138286221: −: ENST00000370603 > chrX: 138072779: −: THE1B | 4 |
| K | | |
| YAGRPLQCL | chr11: 684898: −: ENST00000382409 > chr11: 681362: −: AluSx | 4 |
| IQDGILNPH | | |
| AASCTCAAC | | |
| CDDMTLLLR | | |
| FQTSGLQNC | chr10: 98044785: −: MLT1C > chr10: 98006805: −: ENST00000224337, | 4 |
| SFKRWSMIL | chr10: 98044785: −: MLT1C > chr10: 98006805: −: ENST00000224337 | |
| K | | |
| QLWTLIGM | chr2: 42612922: −: MER34B > chr2: 42580483: −: ENST00000468711 | 4 |
| EMVPEESTL | | |
| TSLGIK | | |
| PAAVALACN | chr1: 234538581: −: AluJb > chr1: 234537008: −: ENST00000040877 | 4 |
| PSTLRGRG | | |
| MPR | | |
| VIHDNFGIV | chr12: 6646556: +: ENST00000229239 > chr12: 6648679: +: MIRb, | 4 |
| EGLMTVTPK | chr12: 6646556: +: ENST00000396856 > chr12: 6648679: +: MIRb, | |
| | chr12: 6646556: +: ENST00000396858 > chr12: 6648679: +: MIRb | |
| EFETSLANM | chr14: 50950778: −: AluSx1 > chr14: 50949134: −: ENST00000013125 | 4 |
| LLDHYQNCK | | |
| AHSTLQDEA | chr19: 17683411: +: ENST00000252599 > chr19: 17684363: +: AluJb | 4 |
| ESFMHVQL | | |
| EVMVR | | |
| HLEEATVAE | chr2: 118715941: −: ENST00000319432 > chr2: 118715754: −: MER58A, | 4 |
| LSSCR | chr2: 118715941: −: ENST00000376300 > chr2: 118715754: −: MER58A | |
| SLNLYNCNS | chr1: 94660048: −: MLT1A0 > chr1: 94655639: −: ENST00000552844 | 4 |
| GAILTTMLA | chr5: 149627335: −: ENST00000348628 > chr5: 149626005: −: MER102b | 4 |
| TRNFSDAVA | | |
| SLDPRSCDS | | |
| R | | |
| QMTQVTM | chr7: 74461534: −: AluJr > chr7: 74457217: −: ENST00000329959 | 4 |
| PGEPVDVAC | | |
| GVDHMVTL | | |
| AK | | |
| QVISAFPTEI | chr3: 51405623: +: L1PA5 > chr3: 51411918: +: ENST00000266037 | 4 |
| STAMPVK | | |
| WVYEVQAG | chr13: 33100704: +: L1MC5 > chr19: 41270986: +: ENST00000243563, | 4 |
| AARDALQG | chr13: 33100704: +: L1MC5 > chr19: 41270986: +: ENST00000601393 | |
| FK | | |
| QVDCLGPG | chr8: 141451863: −: AluJr > chr8: 141449296: −: ENST00000389327 | 4 |
| VPDQPGQH | | |
| DITRSGAK | | |
| YEEASIPFVG | chr1: 182920453: −: ENST00000367547 > chr1: 182915515: −: HAL1, | 4 |
| ILVEK | chr1: 182920453: −: ENST00000423786 > chr1: 182915515: −: HAL1 | |
| QEQSSSPA | chr1: 51946945: −: ENST00000371730 > chr1: 51945188: −: L1PA5 | 4 |
| MEQSWME | | |
| NDFDKLTEL | | |
| GFRR | | |
| LFFLLLTSCR | chr22: 41650398: +: CT−rich > chr22: 41648969: −: ENST00000405486 | 4 |
| AASRGVTFL | chr10: 70722825: +: AluJo > chr10: 70723047: +: ENST00000354185 | 4 |
| FPIQAK | | |
| NPSTLGGQ | chr16: 16171648: +: AluSx > chr16: 16173209: +: ENST00000349029, | 4 |
| ASPSPSPK | chr16: 16171648: +: AluSx > chr16: 16173209: +: ENST00000349029, | |
| | chr16: 16171648: +: AluSx > chr16: 16173209: +: ENST00000351154, | |
| | chr16: 16171648: +: AluSx > chr16: 16173209: +: ENST00000351154, | |
| | chr16: 16171648: +: AluSx > chr16: 16173209: +: ENST00000399410, | |
| | chr16: 16171648: +: AluSx > chr16: 16173209: +: ENST00000399410 | |
| IIQVWWHV | chr7: 116527485: +: AluJo > chr7: 116528181: +: ENST00000361183, | 4 |
| PVVPAAQE | chr7: 116527485: +: AluJo > chr7: 116528181: +: ENST00000426421, | |
| AEVR | chr7: 116527485: +: AluJo > chr7: 116528181: +: ENST00000458284, | |
| | chr7: 116527485: +: AluJo > chr7: 116528181: +: ENST00000490693 | |
| MWSGLPSR | chr2: 180610440: −: ENST00000409343 > chr2: 180493215: −: L1MC5 | 4 |
| GLPIFK | | |
| FLGLGFFICN | chr15: 81281080: −: MIR3 > chr15: 81274523: −: ENST00000561312 | 4 |
| MRR | | |
| AEIAPLHSSL | chr7: 138950208: +: AluSc8 > chr7: 138951079: +: ENST00000288561, | 4 |
| GDRAAK | chr7: 138950208: +: AluSc8 > chr7: 138951079: +: ENST00000483726 | |
| LLPVIPELWE | chr10: 95240482: −: AluSx > chr10: 95216694: −: ENST00000358334 | 4 |
| AEMR | | |
| MDGPAEPQ | chr19: 49055580: +: ENST00000201586 > chr19: 49066561: +: L1ME2z | 4 |
| IPGLWDTYE | | |
| DDISEISYLP | | |
| KR | | |
| NLGSVWWL | chr8: 146016536: −: MIRb > chr8: 146015855: −: ENST00000394920 | 4 |
| EVAELTNPS | | |
| TSLGNMCVI | chr21: 45165696: +: AluJb > chr21: 45165960: +: ENST00000468090 | 4 |
| QSWVTSGT | | |
| AK | | |
| EAANMILVD | chr16: 84488590: +: ENST00000262429 > chr16: 84490583: +: L1HS | 4 |
| DDFSAIIIGK | | |
| MAHAYNPS | chr17: 62480081: −: AluJb > chr17: 62479091: −: ENST00000539111, | 4 |
| TLGSQAPIK | chr17: 62480081: −: AluJb > chr17: 62479091: −: ENST00000581355 | |
| MQVSLHPK | chr8: 105152976: +: L2b > chr8: 105160835: +: ENST00000408894 | 4 |
| CKADK | | |
| GQLRPHTW | chr19: 10200199: +: MIRb > chr19: 10200333: +: ENST00000253110, | 4 |
| LSIRSTR | chr19: 10200199: +: MIRb > chr19: 10200333: +: ENST00000587710, | |
| | chr19: 10200199: +: MIRb > chr19: 10200333: +: ENST00000590378, | |
| | chr19: 10200199: +: MIRb > chr19: 10200333: +: ENST00000593131 | |
| MMKPGLAS | chr7: 37989696: +: L1MB3 > chr7: 37989802: +: ENST00000476620, | 4 |
| IQSRIAILSR | chr7: 37989704: +: L1MB3 > chr7: 37989802: +: ENST00000476620 | |
| WRHGTPA | chr2: 102014362: −: AluSq2 > chr2: 102014186: −: ENST00000376826 | 4 |
| WLIQVLLED | | |
| ETTESAVK | | |
| TDSREAEFP | chr15: 51201329: +: MIRb > chr15: 51204275: +: ENST00000561441 | 4 |
| QKK | | |
| ATVLFYSYKP | chr19: 32948256: +: L4 > chr19: 32949006: +: ENST00000392250, | 4 |
| K | chr19: 32948256: +: L4 > chr19: 32949006: +: ENST00000586987, | |
| | chr19: 32948256: +: L4 > chr19: 32949006: +: ENST00000588648 | |
| LLLSSLSSLG | chr1: 156962904: −: MIRb > chr1: 156955965: −: ENST00000361409, | 4 |
| DSAPER | chr1: 156962904: −: MIRb > chr1: 156955965: −: ENST00000368194 | |
| DLGQVISLD | chr20: 1106051: +: MIR3 > chr20: 1106141: +: ENST00000333082 | 4 |
| LHIFICEVSR | | |
| VPMIR | | |
| ILWWSQGK | chr12: 123839145: −: MIR3 > chr12: 123834988: −: ENST00000267176 | 4 |
| SLGLSFHICII | chr15: 83100895: +: MIRb > chr15: 83103066: +: ENST00000561062 | 4 |
| EAAGRGGE | | |
| AVK | | |
| VISAFPSEVS | chr9: 18711328: +: L1PA3 > chr9: 18721534: +: ENST00000380548 | 4 |
| RR | | |
| MISSTSVYA | chr2: 164591413: −: ENST00000409634 > chr2: 164561904: −: MIR3 | 4 |
| PK | | |
| GLPSHTELS | chr14: 78340168: +: THE1C > chr14: 78353434: +: ENST00000238561 | 4 |
| MICSRASM | | |
| TPLWGRPP | | |
| WPR | | |
| GIPSQVELA | chr7: 81946024: −: THE1B > chr7: 81799925: −: ENST00000356860 | 4 |
| WHWKR | | |
| VTLGISSPVK | chr19: 3368020: +: MIR3 > chr19: 3449012: +: ENST00000341919, | 4 |
| K | chr19: 3368020: +: MIR3 > chr19: 3449012: +: ENST00000346156, | |
| | chr19: 3368020: +: MIR3 > chr19: 3449012: +: ENST00000589123 | |
| ICKAMLELLE | chr10: 5056964: −: PABL_B−int > chr10: 5037645: −: | 4 |
| DPVLCALAK | ENST00000380753 | |
| TCAMAQNI | chr8: 99019829: +: ENST00000521689 > chr8: 99025486: +: Tigger12c, | 4 |
| PEGQKLDILL | chr8: 99019829: +: ENST00000522025 > chr8: 99025486: +: Tigger12c, | |
| NK | chr8: 99019829: +: ENST00000524308 > chr8: 99025486: +: Tigger12c | |
| HVVQGIQII | chr13: 92560311: +: ENST00000377067 > chr13: 92572670: +: HAL1 | 4 |
| R | | |
| VTGSLQDG | chr17: 39976301: +: MIRc > chr17: 39977199: +: ENST00000321562 | 4 |
| MTTGPPRR | | |
| R | | |
| YLSPEAEVA | chr14: 73754128: −: AluSz6 > chr14: 73754022: −: ENST00000554546 | 4 |
| VSRDCATAL | | |
| QPGVK | | |
| QLLCIVSNQ | chr12: 49658051: +: MIRb > chr12: 49663248: +: ENST00000541364, | 4 |
| K | chr12: 49658051: +: MIRb > chr12: 49663248: +: ENST00000552125 | |
| FVSDEMSK | chr20: 33644499: −: L1ME3E > chr20: 33642834: −: ENST00000252015, | 4 |
| DCLSILYNTC | chr20: 33644499: −: L1ME3E > chr20: 33642834: −: ENST00000451813 | |
| VCTEGVTK | | |
| QLEYLARIQ | chr8: 74464247: −: ENST00000517542 > chr8: 74420593: −: Tigger1, | 4 |
| GFQASLFPE | chr8: 74464247: −: ENST00000521210 > chr8: 74420593: −: Tigger1, | |
| TQQY | chr8: 74464247: −: ENST00000521451 > chr8: 74420593: −: Tigger1, | |
| | chr8: 74464247: −: ENST00000521727 > chr8: 74420593: −: Tigger1, | |
| | chr8: 74464247: −: ENST00000522695 > chr8: 74420593: −: Tigger1, | |
| | chr8: 74464247: −: ENST00000523533 > chr8: 74420593: −: Tigger1, | |
| | chr8: 74464247: −: ENST00000523558 > chr8: 74420593: −: Tigger1, | |
| | chr8: 74464247: −: ENST00000524300 > chr8: 74420593: −: Tigger1 | |
| LQGERISGN | chr17: 73725517: +: ENST00000579662 > chr17: 73726063: +: AluJo | 4 |
| LDAPEGGFD | | |
| AILQTAVCT | | |
| K | | |
| LLEILGATIE | chr16: 29692733: +: Charlie4z > chr16: 29708317: +: ENST00000395384, | 4 |
| NSR | chr17: 39696603: −: MIR > chr17: 39681525: −: ENST00000361566, | |
| | chr17: 39696603: −: MIR > chr17: 39681525: −: ENST00000361566 | |
| MTKLLSPIR | chr22: 32787416: −: AluSx1 > chr22: 32784086: −: ENST00000216038 | 4 |
| VLILCQHEAL | chr17: 53344521: +: MIRb > chr17: 53345112: +: ENST00000226067 | 4 |
| VK | | |
| DDSEPGMP | chr12: 56685415: −: L3 > chr12: 56680404: −: ENST00000351328, | 4 |
| VLPPR | chr12: 56685415: −: L3 > chr12: 56680404: −: ENST00000552688 | |
| QWTEILHFT | chr11: 60665318: −: ENST00000227524 > chr11: 60663297: −: Tigger1, | 4 |
| DFPISLDTTIL | chr11: 60665318: −: ENST00000227524 > chr11: 60663297: −: Tigger1, | |
| K | chr11: 60665318: −: ENST00000535326 > chr11: 60663297: −: Tigger1, | |
| | chr11: 60665318: −: ENST00000535326 > chr11: 60663297: −: Tigger1 | |
| MSVLGELS | chr14: 75593608: −: ENST00000238616 > chr14: 75593104: −: MIRc | 4 |
| GQEIKTILAN | chr14: 53079781: +: AluY > chr14: 53211570: +: ENST00000442123, | 4 |
| TEWTYPMR | chr14: 53108136: +: AluY > chr14: 53211570: +: ENST00000442123, | |
| | chr14: 53201596: +: AluY > chr14: 53211570: +: ENST00000442123 | |
| FSSHSEMVII | chr18: 9858290: +: MIRc > chr18: 9859225: +: ENST00000578921 | 4 |
| TTITSNSQG | | |
| EAAR | | |
| TENQTPHVL | chr4: 87716976: +: L1PA6 > chr4: 87718027: +: ENST00000411767 | 4 |
| THRPPK | | |
| QAFLEQTRP | chr3: 127878586: +: L2a > chr3: 127965679: +: ENST00000254730, | 4 |
| LR | chr3: 127878586: +: L2a > chr3: 127965679: +: ENST00000483457, | |
| | chr5: 43583569: −: MER39B > chr5: 43556101: −: ENST00000306846, | |
| | chr5: 43583569: −: MER39B > chr5: 43556101: −: ENST00000306846, | |
| | chr5: 43583569: −: MER39B > chr5: 43556101: −: ENST00000306846 | |
| ESFSDFSSS | chr19: 16201431: +: L2c > chr19: 16204346: +: ENST00000344824 | 4 |
| ILK | | |
| QVLWLMP | chr4: 3344780: +: ENST00000514268 > chr4: 3377116: +: (CCCCAG)n | 4 |
| MIPALSEAE | | |
| AAGSLETSL | | |
| GNMTSAW | | |
| CR | | |
| QTDHLRPG | chr1: 28810817: +: AluSx > chr1: 28815682: +: ENST00000373839, | 4 |
| VQHQSGQH | chr1: 28810817: +: AluSx > chr1: 28815682: +: ENST00000373839, | |
| SK | chr1: 28810817: +: AluSx > chr1: 28815682: +: ENST00000373839 | |
| IEDFGVHCK | chr8: 67968754: −: ENST00000357849 > chr8: 67967541: −: L2a | 4 |
| QMTSHLTK | | |
| YFDYWGQG | chr14: 106330435: −: ENST00000461719 > chr14: 106301002: −: | 4 |
| TLVTQSPPS | MER65−int | |
| K | | |
| AVALYEGTL | chr2: 238451319: +: (TGG)n > chr2: 238454100: +: ENST00000445024 | 4 |
| SLCSEDLK | | |
| MPSLNTSRP | chr22: 22689234: +: L1PA8 > chr22: 22712477: +: ENST00000390294, | 4 |
| SGVPDRFSG | chr22: 22689234: +: L1PA8 > chr22: 22735583: +: ENST00000390297 | |
| SK | | |
| IVGNMFNQ | chr13: 77572426: +: MIRc > chr13: 77574593: +: ENST00000377453 | 4 |
| MAK | | |
| LLIYAAATR | chr2: 90537978: −: ENST00000443397 > chr2: 89942712: −: MLT1D | 4 |
| LFSIHLLSAL | chr4: 55076146: −: L2a > chr2: 89160766: −: ENST00000390240 | 4 |
| GPK | | |
| LCPAATLSE | chr20: 35779434: +: SVA_B > chr20: 35812583: +: ENST00000237530 | 4 |
| R | | |
| TVEQGVGV | chr11: 64042841: +: MIRb > chr11: 64085797: +: ENST00000265462, | 4 |
| WR | chr11: 64042841: +: MIRb > chr11: 64085797: +: ENST00000265462, | |
| | chr11: 64042841: +: MIRb > chr11: 64085797: +: ENST00000347941, | |
| | chr11: 64042841: +: MIRb > chr11: 64085797: +: ENST00000347941 | |
| GLEWIGTK | chr14: 106877762: −: ENST00000390619 > chr14: 106859351: −: MLT1C | 4 |
| QRPLCCDEQ | chr1: 153635838: −: AluJr4 > chr1: 153635752: −: ENST00000361891, | 4 |
| PHQTAFGPK | chr1: 153635838: −: AluJr4 > chr1: 153635752: −: ENST00000361891, | |
| | chr1: 153635838: −: AluJr4 > chr1: 153635752: −: ENST00000361891 | |
| MIVMNDQ | chr10: 64976967: −: ENST00000402544 > chr10: 64445839: −: L2c | 4 |
| MDSSLK | | |
| PGVVAHAC | chr19: 39144970: +: AluSz > chr19: 39191240: +: ENST00000252699, | 4 |
| NPSTLGGRD | chr19: 39144970: +: AluSz > chr19: 39191240: +: ENST00000252699, | |
| LHGMVQLP | chr19: 39144970: +: AluSz > chr19: 39191240: +: ENST00000252699 | |
| PAEGR | | |
| MSPQLLTPG | chr14: 106662989: −: HERVS71−int > chr14: 106641867: −: | 4 |
| SGAHSQVQ | ENST00000390605 | |
| LVQSGAEVK | | |
| LLEGEECRN | chr12: 52910421: −: ENST00000252242 > chr12: 52900057: −: L1MC1 | 4 |
| LASKK | | |
| PGMMAHA | chr6: 31360872: −: AluSg > chr6: 31324570: −: ENST00000412585 | 4 |
| CNPSTLGGR | | |
| GGWIMR | | |
| LFPGQPMV | chr14: 70383468: +: L2a > chr14: 70418855: +: ENST00000361956 | 4 |
| SNK | | |
| TAPPLSPLA | chr14: 51363531: +: LTR7 > chr14: 51368547: +: ENST00000337334 | 4 |
| DSLCGLSPP | | |
| APR | | |
| NLHQSNFSL | chr4: 5827221: −: ENST00000324989 > chr4: 5744561: −: MER66C, | 3 |
| SGEAKYPPR | chr4: 5827221: −: ENST00000397890 > chr4: 5744561: −: MER66C, | |
| | chr4: 5827221: −: ENST00000512574 > chr4: 5744561: −: MER66C | |
| SCIFTISKID | chr6: 74208780: +: MER104 > chr6: 74210297: +: ENST00000415954 | 3 |
| DR | | |
| LHELMERTI | chr1: 212870303: −: ENST00000243440 > chr1: 212869254: −: L2 | 3 |
| FLITQIR | | |
| EAEAGESLE | chr4: 17818661: +: AluSc > chr4: 17818884: +: ENST00000251496 | 3 |
| SDAVKQAM | | |
| QK | | |
| KKADATLLK | chr14: 23350226: +: AluSc > chr14: 23353883: +: ENST00000267396, | 3 |
| | chr14: 23350226: +: AluSc > chr14: 23353883: +: ENST00000536884 | |
| MVEGDQLP | chr1: 183811061: +: Tigger4a > chr1: 183816700: +: ENST00000304685, | 3 |
| PGHTVSQYE | chr1: 183811061: +: Tigger4a > chr1: 183816700: +: ENST00000539189 | |
| TCK | | |
| VCNKNFDD | chr2: 122259789: −: L2a > chr2: 122227536: −: ENST00000263710, | 3 |
| EDSVDGNR | chr2: 122259789: −: L2a > chr2: 122227536: −: ENST00000397587, | |
| PSSASSTSSK | chr2: 122259789: −: L2a > chr2: 122227536: −: ENST00000409078, | |
| | chr2: 122259789: −: L2a > chr2: 122227536: −: ENST00000418989, | |
| | chr2: 122259789: −: L2a > chr2: 122227536: −: ENST00000449975, | |
| | chr2: 122259789: −: L2a > chr2: 122227536: −: ENST00000452274, | |
| | chr2: 122259789: −: L2a > chr2: 122227536: −: ENST00000455322, | |
| | chr2: 122259789: −: L2a > chr2: 122227536: −: ENST00000541377, | |
| | chr2: 122259789: −: L2a > chr2: 122227536: −: ENST00000541859, | |
| | chr2: 122259789: −: L2a > chr2: 122227536: −: ENST00000545861 | |
| MLETLTLISS | chr19: 58288037: +: ENST00000391702 > chr19: 58308975: +: AluSp | 3 |
| LGGLR | | |
| STNVLCTLF | chr2: 131117848: +: L2a > chr2: 131126706: +: ENST00000175756, | 3 |
| MLNLPDLEE | chr2: 131117848: +: L2a > chr2: 131126706: +: ENST00000175756 | |
| K | | |
| QEQSSSPA | chr1: 51946945: −: ENST00000371730 > chr1: 51945188: −: L1PA5 | 3 |
| MEQSWME | | |
| NDFDKLTEL | | |
| GFRR | | |
| MILSSIR | chr6: 144066592: +: L3 > chr6: 144070122: +: ENST00000367584 | 3 |
| MGAVAQAC | chr3: 10237009: +: AluJo > chr3: 10242053: +: ENST00000256458 | 3 |
| NPSTLGGQ | | |
| GKPAPEIR | | |
| YVLAAPEWT | chr10: 22168673: −: Tigger1 > chr10: 22095028: −: ENST00000376980 | 3 |
| EEDLSQLTR | | |
| SMVK | | |
| HPSFRPAM | chr20: 25838731: −: SST1 > chr20: 25755948: −: ENST00000376403 | 3 |
| TVWVGK | | |
| ALEPGRGSE | chr17: 5200809: +: MIRb > chr17: 5235244: +: ENST00000262477 | 3 |
| EAKCSITSCT | | |
| R | | |
| QVLQHPWK | chr3: 118831521: −: L1PA3 > chr3: 118649122: −: ENST00000425327 | 3 |
| SLRPAATPS | chr1: 217697476: −: SVA_D > chr1: 217688231: −: ENST00000366935 | 3 |
| GK | | |
| HRRQISTAM | chr3: 51405599: +: L1PA5 > chr3: 51411918: +: ENST00000266037 | 3 |
| PVK | | |
| TSSGLPLILH | chr7: 33001530: +: MER1A > chr7: 33014229: +: ENST00000242209 | 3 |
| YAMTETPLS | | |
| MCLWEK | | |
| LLDASQFGR | chr1: 243449699: +: ENST00000435549 > chr1: 243451519: +: AluJb | 3 |
| MITR | | |
| SQHFGRPR | chr7: 101810334: +: AluSx1 > chr7: 101813726: +: ENST00000437600 | 3 |
| QVDYLR | | |
| IQNSQVGIA | chr3: 9476785: +: AluSp > chr3: 9477412: +: ENST00000450326 | 3 |
| VQQK | | |
| QPPRPNQL | chr20: 43581385: +: L2b > chr20: 43586984: +: ENST00000372826 | 3 |
| CRR | | |
| LQAEYMAA | chr19: 10561170: +: MIRb > chr19: 10561279: +: ENST00000592685 | 3 |
| SLGHGGPR | | |
| DTVDVLK | chr3: 50089360: +: L1MB7 > chr3: 50091768: +: ENST00000425608 | 3 |
| AASNQISGV | chr2: 90010195: −: LTR62 > chr2: 89246936: −: ENST00000496168 | 3 |
| PSR | | |
| LSWRGSGKI | chr14: 106552518: −: ENST00000390600 > chr14: 106279761: −: L1PA13, | 3 |
| TK | chr14: 106815952: −: ENST00000390615 > chr14: 106279761: −: L1PA13 | |
| QELSILTMQ | chr18: 33758286: +: L1ME1 > chr18: 33775220: +: ENST00000261326 | 3 |
| VPPCSPR | | |
| LSCAASGFT | chr14: 107048875: −: ENST00000390627 > chr14: 107025026: −: L1PB3, | 3 |
| VK | chr14: 107131236: −: ENST00000390632 > chr14: 107025026: −: L1PB3 | |
| QAPGQGLE | chr14: 107170059: −: ENST00000390633 > chr14: 107126752: −: L1ME2, | 3 |
| WMGGIIPIF | chr14: 107170059: −: ENST00000390633 > chr14: 107126752: −: L1ME2 | |
| GTRNYRK | | |
| VCTWTPFN | chr5: 302190: +: (TG)n > chr5: 304292: +: ENST00000505221, | 3 |
| PVTVR | chr5: 302190: +: (TG)n > chr5: 304292: +: ENST00000505221, | |
| | chr5: 302190: +: (TG)n > chr5: 304292: +: ENST00000505221, | |
| | chr5: 302190: +: (TG)n > chr5: 304292: +: ENST00000506909, | |
| | chr5: 302190: +: (TG)n > chr5: 304292: +: ENST00000506909, | |
| | chr5: 302190: +: (TG)n > chr5: 304292: +: ENST00000506909, | |
| | chr5: 302190: +: (TG)n > chr5: 304292: +: ENST00000507473, | |
| | chr5: 302190: +: (TG)n > chr5: 304292: +: ENST00000507473, | |
| | chr5: 302190: +: (TG)n > chr5: 304292: +: ENST00000507473, | |
| | chr5: 302190: +: (TG)n > chr5: 304292: +: ENST00000507528, | |
| | chr5: 302190: +: (TG)n > chr5: 304292: +: ENST00000507528, | |
| | chr5: 302190: +: (TG)n > chr5: 304292: +: ENST00000507528 | |

Importantly, a group of 10 JET-derived proteins were found to be highly recurrent across the dataset, which were also highly reliable according to their MS/MS spectra. In addition, an overexpression of JET-derived proteins was detected in tumor samples annotated as inflammatory, which could be associated to higher interferon-y levels, and therefore an increased expression of transposable elements. These results provide a clear evidence that JETs encode for coding sequences that are translated into protein sequences. Mass spectrometry-based proteomics is thus a useful tool for from the validation of coding JETs.

### Immunopeptidomics

Proteomic analysis provides evidence of the existence of JET-derived proteins within the cell, but it does not elucidate the contribution of those proteins to the repertoire of major histocompatibility complex class I (HLA-I in humans)-associated peptides (also referred to as the immunopeptidome). Presentation by MHC class I molecules (named HLA-I in humans) on the tumor cell surface is indeed required for JET-derived peptides (pJETs) to be recognized by cytotoxic T cells. To demonstrate that JETs can be effectively processed and presented by HLA-I, the presence of lung tumor-specific pJET was searched in MS-based immunopeptidomics datasets (FIG. 16A—Table 8).

TABLE 8

| | | Recurrence |
|---|---|---|
| | | in |
| Sequence | chimeric id | peptidome |
| RATKPTVQK | chr22: 19103521: − > chr22: 19077003: − | 8 |
| MTSQTMVNK | chr17: 34139989: + > chr17: 34147028: + | 5 |
| RTHQQAPLK | chr20: 45967949: − > chr20: 45938948: − | 5 |
| VAAAESHPL | chr9: 136210283: + > chr9: 136216428: + | 5 |
| GIRPPTSVR | chr12: 120659973: − > chr12: 120659561: − | 4 |
| AQMSVPFNR | chr13: 38183213: − > chr13: 38171419: − | 4 |
| SLSPPLVSK | chr19: 46646469: + > chr19: 46663672: + | 4 |
| AVNGKFVGR | chr5: 10384580: + > chr5: 10387106: + | 4 |
| IQNEIHAQR | chr5: 43583569: − > chr5: 43556101: − | 4 |
| RLDHQRAEK | chr1: 180975232: − > chr1: 180974599: − | 3 |
| RWKEVPSVPR | chr10: 123969561: + > chr10: 123969912: + | 3 |
| GVQQYHLLSW | chr10: 72644921: − > chr10: 72642242: − | 3 |
| RLATAPSEK | chr10: 99601433: + > chr10: 99619215: + | 3 |
| YLPDFYFGKF | chr11: 108084796: − > chr11: 108068147: − | 3 |
| TVKTKSLM | chr11: 117024976: + > chr11: 117030650: + | 3 |
| HSKALSLSK | chr11: 118278445: + > chr11: 118279715: + | 3 |
| KTKNLPMQEK | chr11: 19146727: + > chr11: 19164530: + | 3 |
| KTKDLDQFLK | chr12: 41157000: + > chr12: 41312441: + | 3 |
| GGGYNDFGNY | chr12: 54631910: + > chr12: 54677621: + | 3 |
| TEIILRIK | chr14: 75593406: − > chr14: 75593104: − | 3 |
| RMFWIPKTK | chr15: 50744825: + > chr15: 50751197: + | 3 |
| VWMRSPLSTF | chr15: 60657933: − > chr15: 60656722: − | 3 |
| LPDLAGLAHL | chr16: 47626347: + > chr16: 47627411: + | 3 |
| KVLGDVATK | chr2: 171927080: − > chr2: 171917666: − | 3 |
| MVKPPSLLK | chr2: 44554937: + > chr16: 75263893: − | 3 |
| ESFYGIGTTRW | chr5: 140080670: + > chr5: 140081591: + | 3 |
| SIKQLQLLK | chr6: 7300909: − > chr6: 7299056: − | 3 |
| SVVSVLVQK | chr8: 41859766: − > chr8: 41845081: − | 3 |
| ALRAVTLTAK | chr1: 151239815: + > chr1: 151245271: + | 2 |
| YLMPSQLWKA | chr1: 207504641: + > chr1: 207506243: + | 2 |
| EQLDILENI | chr1: 33142971: + > chr1: 33145245: + | 2 |
| RSPDHPRAEY | chr1: 38258152: + > chr1: 38274660: + | 2 |
| ITSWFVPRPW | chr1: 45227052: + > chr1: 45241706: + | 2 |
| NELDKFIVF | chr10: 120938004: − > chr10: 120936665: − | 2 |
| SHKASLNKF | chr10: 47173898: − > chr10: 47139994: − | 2 |
| GTVPKSKAL | chr10: 5058029: − > chr10: 5043873: − | 2 |
| FMMEQVGLAIRS | chr10: 5060092: − > chr10: 5043783: − | 2 |
| MELALRSQL | chr11: 102093793: + > chr11: 102094353: + | 2 |
| RVLLKEELTTPK | chr11: 13465223: − > chr11: 13443385: − | 2 |
| PSSPRPSSL | chr11: 17486945: − > chr11: 17485151: − | 2 |
| GSGKRTSSQLKP | chr11: 44148906: + > chr11: 44151595: + | 2 |
| STSYSISSK | chr11: 61132342: + > chr11: 61133517: + | 2 |
| RTQSSTIQIR | chr11: 61196370: − > chr11: 61189080: − | 2 |
| QEDLGQRGY | chr11: 785039: + > chr11: 810272: + | 2 |
| ETEKDRRPW | chr12: 104681628: + > chr12: 104682709: + | 2 |
| RRLQGATAMVR | chr12: 20704505: + > chr20: 57470710: + | 2 |
| WRFQSCPPTTY | chr12: 53835233: + > chr12: 53836479: + | 2 |
| AVMTEVVLSK | chr12: 54862609: − > chr12: 54857081: − | 2 |
| STSIYSTL | chr12: 56867593: − > chr12: 56867378: − | 2 |
| APKRNTSAL | chr13: 38183213: − > chr13: 38171419: − | 2 |
| RSVSARPPR | chr13: 42456176: − > chr13: 42442613: − | 2 |
| SVAWPFSFPK | chr13: 53015205: − > chr13: 53013303: − | 2 |
| TPNLRTIY | chr13: 76192906: − > chr13: 76104323: − | 2 |
| SIQHSDLLY | chr13: 99992819: + > chr13: 100009388: + | 2 |
| SVKQGTGTSW | chr14: 102196928: + > chr14: 102197694: + | 2 |
| KSQKLREQIK | chr14: 107277425: − > chr14: 106478239: − | 2 |
| RRQQLITRF | chr14: 24686695: − > chr14: 24686429: − | 2 |
| KSQLLERL | chr14: 77914837: − > chr14: 77913291: − | 2 |
| GLFFMKQSV | chr15: 79168112: + > chr15: 79170555: + | 2 |
| RENGFVKKF | chr15: 80254313: − > chr15: 80253516: − | 2 |
| NEQAALSEAKVGGS | chr16: 30778197: + > chr16: 30778878: + | 2 |
| HELQSHHF | chr16: 69773838: − > chr16: 69752437: − | 2 |
| NEIRISEA | chr16: 80584267: + > chr16: 80584412: + | 2 |
| APRMLPIQAV | chr17: 25597844: + > chr17: 25630393: + | 2 |
| EAEVARWL | chr17: 57171332: − > chr17: 57168701: − | 2 |
| RLCPAAPSEK | chr17: 67192427: − > chr17: 67190636: − | 2 |
| APRGPSGTQEM | chr19: 3624569: − > chr19: 3624160: − | 2 |
| STHPPPPSGL | chr19: 3782560: − > chr19: 3781670: − | 2 |
| LYSRYKKLQQHSGAK | chr19: 40478151: + > chr19: 40491610: + | 2 |
| AAAVNQGLSY | chr19: 40540857: − > chr19: 40534017: − | 2 |
| LWPSPVNLRF | chr19: 42217314: + > chr19: 42311149: + | 2 |
| VSLSPPLVSK | chr19: 46646469: + > chr19: 46663672: + | 2 |
| VAAAAARVL | chr19: 8323741: + > chr19: 8326572: + | 2 |
| WRTSALGQR | chr2: 158306719: − > chr2: 158300518: − | 2 |
| KYKLLDIGYRK | chr2: 224817241: + > chr2: 224824251: + | 2 |
| KFTRFFVK | chr2: 24029041: − > chr2: 24021193: − | 2 |
| LHQKGGITF | chr2: 24198120: + > chr2: 24199839: + | 2 |
| QPSQTQVPL | chr2: 28859785: + > chr2: 28863793: + | 2 |
| ETFPGKSTF | chr2: 30856371: + > chr2: 30862983: + | 2 |
| DVLDKFMSF | chr2: 70488518: + > chr2: 70489942: + | 2 |
| GRWITPSNR | chr2: 89166769: − > chr2: 89157082: − | 2 |
| SAALQALKR | chr20: 32408074: + > chr20: 32436273: + | 2 |
| TATQRPTPM | chr20: 34710669: + > chr20: 34761806: + | 2 |
| RLLVPSSPK | chr20: 44540197: − > chr20: 44540102: − | 2 |
| TEATVDTSF | chr20: 60770037: + > chr20: 60770858: + | 2 |
| LASREAIL | chr20: 9457400: + > chr20: 9484605: + | 2 |
| HDLVRISM | chr21: 27105478: − > chr21: 27102112: − | 2 |
| NPQFLISMILQR | chr21: 27105526: − > chr21: 27102112: − | 2 |
| MRATKPTVQK | chr22: 19103521: − > chr22: 19077003: − | 2 |
| SAAAGIVGR | chr22: 39716401: − > chr22: 39714597: − | 2 |
| HTMGFWLTK | chr3: 10160654: + > chr3: 10188198: + | 2 |
| SPRDEGKVFR | chr3: 120840551: + > chr3: 120866822: + | 2 |
| QALYCQLLTY | chr3: 132948195: + > chr3: 132969129: + | 2 |
| GTFPFAFSL | chr3: 185198511: − > chr3: 185194935: − | 2 |
| KTFPLSKSPK | chr4: 106617004: − > chr4: 106616825: − | 2 |
| KVLEKYWLYK | chr4: 123834362: − > chr4: 123833778: − | 2 |
| TTDAAIRY | chr5: 10384580: + > chr5: 10387106: + | 2 |
| QAQPTLRPPK | chr5: 133406533: − > chr5: 133326845: − | 2 |
| AQPTLRPPK | chr5: 133406533: − > chr5: 133326845: − | 2 |
| KPAVARAR | chr5: 134415207: − > chr5: 134367163: − | 2 |
| ESFYGIGTTR | chr5: 140080670: + > chr5: 140081591: + | 2 |
| RRNQETGAVQR | chr5: 1503092: − > chr5: 1501718: − | 2 |
| ATDNDFSRRNLY | chr5: 1503092: − > chr5: 1501718: − | 2 |
| EVVSTSYRR | chr5: 87546068: − > chr5: 87524345: − | 2 |
| KLRDPTDSTLR | chr6: 109427293: + > chr6: 109466422: + | 2 |
| SPSADRVVAL | chr6: 43395970: + > chr6: 43399880: + | 2 |
| KVSSPESGLSNR | chr7: 101514832: + > chr7: 101559395: + | 2 |
| DIISAFHPF | chr7: 103845571: − > chr7: 103844682: − | 2 |
| AVFSLRPIPR | chr7: 32555261: + > chr7: 32582753: + | 2 |
| GRFPSQAQL | chr7: 40618566: + > chr7: 40789033: + | 2 |
| DIFQQQISR | chr7: 56036577: + > chr7: 56045819: + | 2 |
| SVSATSVQK | chr9: 101867407: + > chr9: 101891137: + | 2 |
| VHCDPTKGF | chr9: 71770431: + > chr9: 71827464: + | 2 |
| IVQHTEISSK | chrX: 122865506: − > chrX: 122846758: − | 2 |

HLA-I peptidomics identified 116 tumor-specific pJETs across 17 primary lung tumors and 2 tumor cell lines (one of the two cell lines was treated with interferon gamma). Interestingly, some pJETs were found in more than 1 sample, indicating that they are shared epitopes. Importantly, pJETs showed similar MS/MS identification scores and peptide length distributions as the annotated peptidome (FIGS. 16B and C). To further validate the reliability of the identifications, MS/MS spectra were manually verified, and 8 of them were confirmed with MS/MS spectra of the corresponding synthetic peptides. These results fully confirmed that the present method if reliable for the identification of pJETS that are presented by HLA-I molecules on tumor cells and are therefore accessible to cytotoxic cells.

### Methods

### Total Proteomics

For the discovery of JETs (junctions exon-transposable element) at the proteome level, two sources of mass spectrometry datasets were used. Firstly, a publicly available dataset of 504 mass spectrometry raw files corresponding to 375 cell lines of the CCLE (Cancer Cell Line Encyclopedia) were used. The original study associated to these analyses was described and published by Nusinow and colleagues in Cell in 2020 (DOI: 10.1016/j.cell.2019.12.023). The second source of data was lung primary tumours obtained from Stewart et al. in Cell 2019 (raw files downloaded from PRIDE database-accession code PXD010357).

Briefly, in both datasets, total protein extracts were obtained from cell lines or tumours and were subsequently digested with trypsin. The resulting peptides were chemically labelled with isobaric tags using TMT, where different samples were analysed in the same experiment, together with an internal reference standard. Peptides were fractionated offline through HPLC and different fractions from each experiment were run on the mass spectrometer separately (Orbitrap Fusion or Orbitrap Fusion Lumos, from Thermo-Fisher). Further details regarding the experimental procedures and analysis are available in the corresponding publications.

Raw output files from mass spectrometry runs were interrogated using Proteome Discoverer 2.4 (Thermo-Fisher), with Sequest-HT as search engine. Two customized databases were used to query the mass spectrometry peaks, both of them including Swissprot and TrEMBL canonical sequences, as well as the in silico translation of chimeric sequences predicted from different datasets. One of databases, the “CCLE-recurrent”, included those JETs found in at least 7 samples of the CCLE mRNA-seq collection. The other database, the “lung-specific”, was constructed adding to the canonical sequences those tumor-specific JETs detected on lung cell lines from CCLE. Thus, two different outputs were obtained, according to the library of predictions set as reference. Protein cleavage was specified as Trypsin allowing for a maximum of 2 miss-cleavages. Peptide FDR was set to 1% while protein FDR was allowed to 100%, to focus our search on the investigation of peptides. The mass tolerance for peptides was 4.5 ppm and fragment tolerance 0.02 Da. Carbamidomethylation of Cysteines was set as fixed modification. For the quantification, signals from TMT reporters were obtained using MS2 or MS3 fragmentation, paired with the MS2 scans for peptide identification.

### Cell Lysis and HLA-ABC-Peptide Complexes Purification

Dry pellets from H1650 cells were resuspended in lysis buffer and sonicated. The sample was centrifuged 12 min at 5500 g and 4° C. to remove nuclei and organelles. The supernatant was collected and ultracentrifuged at 72000 g for 1 h at 4° C. to extract the membranes and the pellet was resuspended with solubilization buffer. After overnight incubation at 4° C., the sample was ultracentrifuged at 55000 g for 1 h at 4° C. to pellet the non-solubilized membranes. Solubilized membranes were incubated overnight with CNBr-activated Sepharose beads (GE Healthcare Life Sciences) coupled to anti-HLA-ABC W6/32 antibody. After washing, peptide-HLA-ABC complexes were eluted with 0.25% TFA. The eluted material was cleaned by a C18 tip before mass spectrometry. Eluted samples were analyzed by Liquid Chromatography-Mass spectrometry (LC-MS) using an Orbitrap Fusion Lumos™ Tribrid (ThermoFisher) equipped with a nanoESI source and coupled to a nanochromatographic system. LC separation was done using a 140-min acetonitrile gradient. Analyses were performed in a Top Speed (most intense) data-dependent mode using a Higher-energy Collisional Dissociation (HCD).

### Immunopeptidomics

Mass spectrometry output files (called raw files) were downloaded from PRIDE database (dataset identifiers: PXD013649, MSV000082648, PXD009752, PXD009754, PXD009755 and PXD009936) or generated in house (for the H1650 cell line). Raw files were processed using ProteomeDiscoverer 2.4 (ThermoFisher) with the following parameters: no-enzyme, precursor mass tolerance 20 ppm and fragment mass tolerance 0.02 Da. Methionine and N-acetylation were enabled as variable modifications. Using Percolator, a false discovery rate (FDR) of 1% was applied at peptide level and no FDR was used at protein level. MS/MS spectra were searched against the human proteome from Uniprot/SwissProt with isoforms concatenated with the in-silico translated lung tumour-specific JETs.

Identified peptides in each sample were processed individually to GibbsCluster 2.0 Server and each cluster was attributed to a HLA-I allele. Only peptides grouped to a given cluster were kept for further analyses. To ensure that JET-derived peptides are not found in canonical proteins, identified peptides were filtered with UniProt/TrEMBL database. Leucine and isoleucine were treated as equivalent. Remaining sequences were aligned to the translated junction using our own custom R scripts. Only those peptides overlapping the junction or in gene frameshift were kept. Finally, spectrums from identified peptides were checked manually.

Synthetic peptides (HPLC purity of 95%) were injected in a LTQ Orbitrap and/or in a Orbitrap Fusion Lumos (CID/HCD). Raw files were analysed with ProteomeDiscoverer 2.5 (ThermoFisher). Spectrums were exported and compared to the endogenous peptide using Msnbase R package. Only PSM with the same charge between synthetic and endogenous and without modifications were analysed.

The following tables 9 and 10 respectively refer to the detailed identification of the neoantigenic peptides of SEQ ID NO 1 to 10170 translated from fusion transcripts wherein the exon is the donor and the neoantigenic peptides of SEQ ID NO 10171 to 38759 translated from fusion transcripts wherein the TE is the donor.

The column named “position” refers to the various chimeric proteins (identified by their SEQ ID NO) that are derived from splice variants of the same JET (or fusion). In table 10, the SEQ ID NO of the chimeric protein can be obtained by adding 10170 to the number(s) provided. For example, in line 2, the column “position” refers to 4 chimeric proteins of SEQ ID NO:10171 to 10174

The last column in each table gives the position of the breakpoint between the exon-derived aa sequence and the TE-derived aa sequence respectively for each of the chimeric proteins (if more than one) of the previous column (position).

In the following tables 11-20, columns 1-13 refer to the following items

| | Fusions (donor: exon) | Fusions (donor: TE) |
|---|---|---|
| 1 | Fusion_id | Fusion_id |
| 2 | Donor_Chromosome_Exon | Donor_Chromosome_TE |
| 3 | Donor_start_Exon | Donor_start_TE |
| 4 | Donor_Breakpoint_Exon | Donor_Breakpoint_TE |
| 5 | Donor_tx_name_Exon | Donor_tx_name_TE |
| 6 | Donor_strand_Exon | Donor_strand_TE |
| 7 | Acceptor_Chromosome_TE | Acceptor_Chromosome_exon |
| 8 | Acceptor_Breakpoint_TE | Acceptor_Breakpoint_exon |
| 9 | Acceptor_end_TE | Acceptor_end_exon |
| 10 | Acceptor_strand_TE | Acceptor_strand_exon |
| 11 | Chimeric protein (i.e. translated JET) reference | Chimeric protein (i.e. translated JET) |
| | | reference |
| 12 | Breakpoint_position_in_AA | Breakpoint_position_in_AA |
| 13 | Tissue | Tissue |

Column 11 refers to the various chimeric proteins (identified by their SEQ ID NO) that are derived from splice variants of the same JET (or fusion).
In table 11, the SEQ ID NO of the chimeric protein can be obtained by adding 39596 to the number(s) provided.
In table 12, the SEQ ID NO of the chimeric protein can be obtained by adding 39596 to the number(s) provided.
In table 13, the SEQ ID NO of the chimeric protein can be obtained by adding 39923 to the number(s) provided.
In table 14, the SEQ ID NO of the chimeric protein can be obtained by adding 39923 to the number(s) provided.
I In table 15, the SEQ ID NO of the chimeric protein can be obtained by adding 40127 to the number(s) provided.
In table 16, the SEQ ID NO of the chimeric protein can be obtained by adding 40127 to the number(s) provided.
In table 17, the SEQ ID NO of the chimeric protein can be obtained by adding 40364 to the number(s) provided.
In table 18, the SEQ ID NO of the chimeric protein can be obtained by adding 40364 to the number(s) provided.
In table 19, the SEQ ID NO of the chimeric protein can be obtained by adding 40509 to the number(s) provided.
In table 20, the SEQ ID NO of the chimeric protein can be obtained by adding 40509 to the number(s) provided.

### Description of the Sequences

| SEQ ID NO: | Sequence description |
|---|---|
| 1-10170 | Translated peptides from transcripts table 9: exon donor |
| 10171-38759 | Translated peptides from transcripts table 10: TE donor |
| 38760-38876 | LUAD peptides of table 3 (117) |
| 38877-38907 | Additional peptides from FIGS. 11-16 (31) |
| 38908-38915 | CDRs sequences table 4bis (8) |
| 38916-39104 | Table 5 peptides from MS (189) |
| 39105-39315 | Table 6 peptides from MS (211) |
| 39316-394820 | Table 7 peptides from MS (167) |
| 39483-39596 | Table 8 peptides from MS (116) |
| 39597-39683 | Translated peptides (JETs of table 11: exon donor) (87) |
| 39684-39923 | Translated peptides (JETs of table 12: TE donor) (240) |
| 39924-39965 | Translated peptides (JETs of table 13: exon donor) (42) |
| 39966-40177 | Translated peptides (JETs of table 14: TE donor) (162) |
| 40128-40193 | Translated peptides (JETs of table 15: exon donor) (66) |
| 40194-40364 | Translated peptides (JETs of table 16: TE donor) (171) |
| 40365-40391 | Translated peptides (JETs of table 17: exon donor) (27) |
| 40392-40509 | Translated peptides (JETs of table 18: TE donor) (118) |
| 40510-40728 | Translated peptides (JETs of table 19: exon donor) (219) |
| 40729-41099 | Translated peptides (JETs of table 20: TE donor) (371) |

1. An isolated tumor neoantigenic peptide comprising at least 4, 5, 6, 7 or 8 amino acids of any one of SEQ ID NO: 1-38907 and 38916-41099.

2. An isolated tumor neoantigenic peptide, according to claim 1 wherein (a) the peptide is from any one of SEQ ID NO:1-38907 and 38916-41099 or a fragment thereof, and comprises at least a portion of TE-derived amino acid sequence from any one of SEQ ID NO:1-38907 and 38916-41099, optionally (i) a fragment that overlaps the breakpoint between, the TE-derived amino acid sequence and an exon-derived amino acid sequence or, optionally (ii) a pure TE sequence; or (b) the peptide is from any one of SEQ ID NO: 1-10170; 38760-38907; 38916-39683; 39924-39965; 40128-40193; 40365-40391; and 40510-40728 or a fragment thereof, and is encoded by a non-canonical ORF downstream of the junction between the TE-derived amino acid sequence and the exon-derived amino acid sequence.

3. The isolated tumor neoantigenic peptide according to claim 1, wherein said neoantigenic peptide
is expressed at higher levels in tumor cells compared to normal healthy cells;
is expressed in at least 1% of subjects from a population of subjects suffering from cancer; and/or
binds MHC class I or class II with a Kd binding affinity of less than about 10⁻⁵ M.

4. A population of autologous dendritic cells or antigen presenting cells that have been pulsed with one or more of the peptides as defined in claim 1 or transfected with a polynucleotide encoding one or more of the peptides as defined in claim 1.

5. A vaccine or immunogenic composition capable of raising a specific T-cell response comprising
a. one or more neoantigenic peptides as defined in claim 1, optionally with a physiologically acceptable buffer, carrier, or excipient, and/or optionally with an adjuvant or immunostimulant;
b. one or more polynucleotides encoding a neoantigenic peptide as defined in claim 1, optionally linked to a heterologous regulatory control nucleotide sequence; and/or
c. a population of antigen presenting cells that have been pulsed with one or more of the peptides as defined in claim 1 or transfected with a polynucleotide encoding one or more of the peptides as defined in claim 1.

6. An antibody, or an antigen-binding fragment thereof, a T cell receptor (TCR), or a chimeric antigen receptor (CAR) that specifically binds a neoantigenic peptide as defined in claim 1, optionally in association with an MHC molecule, with a Kd affinity of about 10⁻⁶ M or less, optionally wherein the antibody is a TCR-like antibody or the CAR is a TCR-like antibody-based CAR.

7. A method of producing an antibody, TCR or CAR that specifically binds a neoantigenic peptide as defined in claim 1, comprising the step of selecting an antibody, TCR or CAR that binds to a tumor neoantigen peptide of claim 1, optionally in association with an MHC or HLA molecule, or optionally expressed on the surface of a cell, with a Kd binding affinity of about 10⁻⁶ M or less.

8. An antibody, TCR or CAR produced by the method of claim 7.

9. A T cell receptor according to claim 8, wherein said T cell receptor is made soluble or a TCR-like antibody fused to an antibody fragment directed to a T cell antigen, optionally wherein the targeted antigen is CD3 or CD16.

10. An antibody, TCR or CAR according to claim 6, wherein said antibody is a multispecific antibody that further targets at least an immune cell antigen, optionally wherein the immune cell is a T cell, a NK cell or a dendritic cell, optionally wherein the targeted antigen is CD3, CD16, CD30 or a TCR, optionally wherein the immune cell is defective for the Suv39h1 gene.

11. A polynucleotide encoding a neoantigenic peptide as defined in claim 1 or a polynucleotide encoding an antibody, a CAR or a TCR, which specifically binds a neoantigenic peptide as defined in claim 1, optionally in association with an MHC molecule, with a Kd affinity of about 10⁻⁶ M or less, optionally wherein the antibody is a TCR-like antibody or the CAR is a TCR-like antibody-based CAR;
optionally linked to a heterologous regulatory control sequence.

12. A vector comprising the polynucleotide of claim 11.

13. An immune cell that specifically binds to one or more neoantigenic peptides as defined in claim 1, optionally wherein the immune cell is defective for the Suv39h1 gene.

14. The immune cell of claim 13, which is an allogenic or autologous cell selected from T cells, Natural Killer T cells, CD4+/CD8+ T cells, TILs/tumor derived CD8 T cells, central memory CD8+ T cells, Treg, MAIT, Yδ T cells, human embryonic stem cells, and pluripotent stem cells from which lymphoid cells may be differentiated.

15. A T cell according to claim 14, which comprises
a T cell receptor that specifically binds one or more neoantigenic peptides as defined in claim 1, or
a TCR or a CAR that specifically binds to one or more neoantigenic peptides as defined in claim 1, optionally wherein the CAR is an MHC-restricted antibody-based chimeric antigen receptor.

16. A method of inhibiting cancer cell proliferation, a method of cancer vaccination therapy of a subject or a method of treating cancer in a subject, comprising administering to a subject in need thereof the neoantigenic peptide as defined in claim 1, a population of dendritic cells or antigen presenting cells that bind to one or more of the peptides as defined in claim 1, a vaccine or immunogenic composition capable of raising a specific T-cell response to one or more of the peptides of claim 1, a polynucleotide encoding a neoantigenic peptide as defined in claim 1 or a polynucleotide encoding an antibody, a CAR or a TCR which specifically binds a neoantigenic peptide as defined in claim 1, a vector comprising said polynucleotide or an antibody, a CAR or a TCR which specifically binds the neoantigenic peptide as defined in claim 1.

17. (canceled)

18. A method of treating cancer in a subject, comprising administering to a subject in need thereof a n immune cell that specifically binds to one or more neoantigenic peptides as defined in claim 1, optionally wherein the immune cell is defective for the Suv39h1 gene, optionally wherein the immune cell is an allogenic or autologous cell selected from T cells, Natural Killer T cells, CD4+/CD8+ T cells, TILs/tumor derived CD8 T cells, central memory CD8+ T cells, Treg, MAIT, γδ T cells, human embryonic stem cells, and pluripotent stem cells from which lymphoid cells may be differentiated, and optionally wherein the immune cell comprises a TCR or CAR that specifically binds a neoantigenic peptide as defined in claim 1.

19. The method of claim 16 comprising administering at least one further therapeutic agent, optionally a chemotherapeutic agent, an immunotherapeutic agent, or a checkpoint inhibitor.

20. The method of claim 18 comprising administering at least one further therapeutic agent, optionally a chemotherapeutic agent, or an immunotherapeutic agent, or a checkpoint inhibitor.
